SEQUENCE LISTING (3 sequences)

SEQ ID NO: 1 - protein, 76 aa, synthetic construct
  REGION 1..76: note = Description of sequence: Adropin consesus sequence
MGAAISQGALIAIVCNGLVGFLLLLLWVILCWACHSRSADVDSLSESSPNSSPGPCPEKAPPPQKPSHEGSYLLQP

SEQ ID NO: 2 - protein, 43 aa, synthetic construct
  REGION 1..43: note = Description of sequence: Adropin consesus sequence
CHSRSADVDSLSESSPNSSPGPCPEKAPPPQKPSHEGSYLLQP

SEQ ID NO: 3 - DNA, 1028 bp, synthetic construct
  misc_feature 1..1028: note = Description of sequence: ENHO consesus sequence
  source 1..1028: mol_type = other DNA
agaccgcccgcggcagaggccgccccggtcgcgccgcggcgggagcggccggtggaggctgcgcggccgagggggagggccgggggagcggacgtcgcctctgctggtctcccacctcccgccgccccccgcccgcaggctcccaagccttagtcggcgccgagcatcccgctgccccggaccctcccgcgggcgcgcaccaggctcaactcaggctcaggactgcaggtagacatctccactgcccaggaatcactgagcgtgcagacagcacagcctcctctgaaggccggccataccagagtcctgcctcggcatgggcctcaccattgaggcagctccactgtctgtgctggtctgagggtgctgcctgtcatgggggcagccatctcccagggggccctcatcgccatcgtctgcaacggtctcgtgggcttcttgctgctgctgctctgggtcatcctctgctgggcctgccattctcgctctgccgacgttgactctctctctgaatccagtcccaactccagccctggcccctgtcctgagaaggccccaccaccccagaagcccagccatgaaggcagctacctgctgcagccctgaaggcccctggcctagcctggagcccaggacctaagtccacctcacctagagcctggaattaggatcccagagttcagccagcctggggtccagaactcaagagtccgcctgcttggagctggacccagcggcccagagtctagccagcttggctccaataggagctcagtggccctaaggagatgggcctggggtgggggcttatgagttggtgctagagccagggccatctggactatgctccatcccaagggccaagggtcaggggccgggtccactctttccctaggctgagcacctctaggccctctaggctggggaagcaaactggaacccatggcaataataggagggtgtccaggctgggcccctcccctggtcctcccagtgtttgctggataataaatggaactatggctcta

(19) United States
(12) Patent Application Publication
(10) Pub. No.: US 2026/0091136 A1
(43) Pub. Date: Apr. 2, 2026

(54) USING ENHO GENE THERAPY TO TREAT CARDIOMETABOLIC DISEASE

(71) Applicant: University of Pittsburgh-of the Commonwealth System of Higher Education, Pittsburgh, PA (US)

(72) Inventor: Iain Scott, Pittsburgh, PA (US)

(73) Assignee: University of Pittsburgh-of the Commonwealth System of Higher Education, Pittsburgh, PA (US)

(21) Appl. No.: 19/407,768

(22) Filed: Dec. 3, 2025

Related U.S. Application Data
(63) Continuation of application No. PCT/US2024/035052, filed on Jun. 21, 2024.
(60) Provisional application No. 63/509,971, filed on Jun. 23, 2023.
(60) Provisional application No. 63/593,067, filed on Oct. 25, 2023.
(60) Provisional application No. 63/575,255, filed on Apr. 5, 2024.

(51) Int. Cl. A61K 48/00; A61K 9/00; A61K 38/22; A61P 9/04; C12N 15/86

(52) U.S. Cl. CPC A61K 48/005; A61K 9/0019; A61K 38/22; A61K 48/0075; A61P 9/04; C12N 15/86; C12N 2750/14143

(57) ABSTRACT

Methods for preventing and/or treating a cardiometabolic disease, e.g., heart failure with preserved ejection fraction (HFpEF) using Adropin are provided herein. The presently disclosed subject matter further relates to gene therapy methods for providing endogenous production of Adropin.

### CROSS-REFERENCE TO RELATED APPLICATIONS

This application is a continuation of International Application No. PCT/US2024/035052, filed Jun. 21, 2024, which claims priority to U.S. Provisional Application No. 63/509,971, filed Jun. 23, 2023, U.S. Provisional Application No. 63/593,067, filed Oct. 25, 2023, and U.S. Provisional Application No. 63/575,255, filed Apr. 5, 2024, priority to each of which is claimed, and the contents of each of which are incorporated by reference in their entireties.

### GRANT INFORMATION

This invention was made with government support under DK13408, OD023402, OD032141, HL147861, HL156874, CA047904, and OD023684 awarded by the National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Dec. 3, 2025, is entitled “072396.1119_ST26.xml”, and is 6,131 bytes in size.

### INTRODUCTION

The presently disclosed subject matter relates to the use of Adropin for the prevention and/or treatment of cardiometabolic disease, e.g., heart failure with preserved ejection fraction (HFpEF). The presently disclosed subject matter further relates to gene therapy methods for providing endogenous production of Adropin.

### BACKGROUND

Cardiometabolic disease describes a range of pathologies related to insulin resistance, dyslipidemia, metabolic syndrome, and cardiac dysfunction. Deleterious changes in whole-body and/or tissue-specific energy metabolism drive the development of cardiometabolic disease, and addressing this defect is a potential novel therapeutic approach.

ENHO gene regulation represents a novel therapeutic target, leading to the production of the metabolic regulatory peptide Adropin. Full-length Adropin is endogenously produced in liver hepatocytes, and a cleaved bioactive version of the peptide (Adropin³⁴⁻⁷⁶) is secreted into the systemic circulation to act on other peripheral tissues. In healthy populations Adropin levels are high; however, there is a significant drop in circulating levels of the peptide in patients with cardiometabolic disease or aging. Previous published work from our group has shown that: (i) Adropin treatment restores glucose oxidation in the hearts of diabetic mice; and (ii) Adropin treatment decreases liver glucose production in diabetic mice.

Exogenous recombinant Adropin peptide is rapidly degraded by endogenous proteases following administration; thus, repeated treatments are needed. The need for repeated treatments due to rapid degradation of exogenous recombinant Adropin peptide limits its therapeutic use. There is need for a gene therapy approach which can provide more consistent therapeutic outcomes.

### SUMMARY OF THE INVENTION

The presently disclosed subject matter provides methods for preventing and/or treating a subject having cardiometabolic disease, comprising administering a therapeutically effective amount of an Adropin-based therapy. In certain embodiments, the cardiometabolic disease is selected from the group consisting of heart failure with preserved ejection fraction (HFpEF), type-2 diabetes, hypertension, or non-alcoholic fatty liver disease (NAFLD). In certain embodiments, the Adropin-based therapy is administered intravenously, intraperitoneally, or subcutaneously.

In certain embodiments, the Adropin-based therapy is a composition comprising a virus wherein the virus comprises a heterologous nucleic acid encoding an Energy Homeostasis-associated (ENHO) gene. In certain embodiments, the virus is administered into the liver of the subject. In certain embodiments, the virus is administered to hepatocytes of the subject. In certain embodiments, the heterologous nucleic acid is operably linked to a promoter selected from the group consisting of thyroxine binding globulin (TBG), albumin (ALB), hepatitis virus (HBV), alpha-1 antitrypsin (AAT), and human cytomegalovirus (CMV). In certain embodiments, the virus is an adenoviral associated virus (AAV). In certain embodiments, the AAV isotype is selected from the group consisting of AAV1, AAV2, AAV3, AAV6, AAV6.2, AAV7, AAV8, AAV9, AAVrh10, AAV/DJ, and AAV/DJ8. In certain embodiments, the ENHO gene comprises SEQ ID NO.: 3.

In certain embodiments, the Adropin-based therapy is a recombinant Adropin peptide. In certain embodiments, the recombinant Adropin peptide comprises SEQ ID NO.: 1 or SEQ ID NO.: 2. In certain embodiments, the recombinant Adropin peptide is administered in an amount between about 1 ng/kg to about 200 mg/kg.

The presently disclosed subject matter further provides a pharmaceutical composition comprising a therapeutically effective amount of an Adropin-based therapy. In certain embodiments, the pharmaceutical composition further comprises a pharmaceutically acceptable carrier. In certain embodiments, the Adropin-based therapy is a virus, wherein the virus comprises a heterologous nucleic acid encoding an ENHO gene. In certain embodiments, the virus is an AAV. In certain embodiments, the ENHO gene comprises SEQ ID NO.: 3. In certain embodiments, the Adropin-based therapy is a recombinant Adropin peptide. In certain embodiments, the recombinant Adropin peptide comprises SEQ ID NO.: 1 or SEQ ID NO.: 2.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIGS. 1A-1B show that four weeks of daily Adropin treatment leads to a decrease in body weight in HFpEF mice. FIG. 1A shows daily body weight through four weeks.

FIG. 1B shows final total body weight from FIG. 1A, with changes marked reflecting relative increase or decrease in body weight per mouse in each group between days 0 and 29 of the study. One-way ANOVA with Tukey post-hoc; ****=p-value<0.0001.

FIGS. 2A-2E show that two weeks of daily Adropin treatment improves diastolic function in HFpEF mice. FIGS. 2A-2E show measurements for left ventricle (LV) mass (FIG. 2A), cardiac output (FIG. 2B), ejection fraction (FIG. 2C), isovolumetric relaxation time (IVRT) (FIG. 2D), and left ventricle myocardial performance index (LV MPI) (FIG. 2E). One-way ANOVA with Tukey post-hoc; *=p-value<0.05; ***=p-value<0.001.

FIGS. 3A-3C show that four weeks of daily Adropin treatment reduces cardiac hypertrophy in HFpEF mice. FIGS. 3A-3C show differences in mass for the heart (FIG. 3A), lung (FIG. 3B), and liver (FIG. 3C). One-way ANOVA with Tukey post-hoc; *=p-value<0.05.

FIGS. 4A-4B show that four weeks of Adropin treatment leads to an increase in glucose tolerance in HFpEF mice. FIG. 4A shows glucose tolerance testing. FIG. 4B shows AUC of FIG. 4A. One-way ANOVA with Tukey post-hoc; *=p-value<0.05; ***=p-value<0.001.

FIGS. 5A-5D show that Adropin-mediated improvement of diastolic function is GPR19-dependent. FIG. 5A shows fasted body weight of mice following glucose tolerance testing. Analysis was conducted for wildtype and GPR19-knockout mice receiving normal chow, HFpEF diet, or HFpEF diet with Adropin treatment. FIGS. 5B-5D show differences in ejection fraction (FIG. 5B), the ratio between the early mitral valve inflow velocity (MV E) and the mitral annular early diastolic velocity (MV e′) (FIG. 5C), and IVTR (FIG. 5D). One-way ANOVA with Tukey post-hoc; *=p-value<0.05; **=p-value<0.01; ***=p-value<0.001; ****=p-value<0.0001.

FIG. 6 shows the impact of Adropin treatment on cardiac dysfunction in HFpEF mice. HFpEF mice developed cardiac dysfunction, as evidenced by increased heart mass, decreased cardiac output, increased isovolumetric relaxation time (IVRT), and increased left ventricular myocardial performance index (LV MPI). Diastolic function is improved in HFpEF following Adropin treatment. One-way ANOVA with Tukey's multiple comparison test.

FIGS. 7A-7B show that Adropin treatment prevented the development of fibrosis and cardiomyocyte hypertrophy in mice exposed to a HFpEF diet. FIG. 7A shows that cardiac fibrosis is reduced in HFpEF mice following Adropin treatment. FIG. 7B shows that cardiomyocyte hypertrophy is reduced in HFpEF mice following Adropin treatment. One-way ANOVA with Tukey's multiple comparison test.

FIGS. 8A-8B show the impact of Adropin treatment on body weight and glucose tolerance. FIG. 8A shows that Adropin treatment does not significantly change absolute body weight between HFpEF and HFpEF+Adropin-treated mice. These measures reflect absolute differences in body weight between groups at study day 28. This analysis is provided to contrast with the analysis of the same time course data in FIG. 1B, which shows the relative change in body weight per mouse in each group between study day 0 and day 29. FIG. 8B shows that Adropin treatment significantly improves whole-body glucose sensitivity. One-way ANOVA with Tukey's multiple comparison test.

FIGS. 9A-9B show transcriptomic and metabolomic analyses of hearts from control HFpEF and Adropin treated HFpEF mice. FIG. 9A shows that the majority of genes downregulated by Adropin treatment are related to fibrosis/extracellular matrix. FIG. 9B shows that hexosamine biosynthesis pathways (HBP) are modulated by Adropin treatment.

FIG. 10 shows that Adropin treatment inhibited several steps in hexosamine biosynthesis pathways (HBP) to decrease total cardia O-GlcNAcylation. One-way ANOVA with Tukey's multiple comparison test.

FIG. 11 shows that Adropin treatment reverses excessive O-GlcNAcylation of FAO enzymes in HFpEF. O-GlcNAcylation was reduced for the mitochondrial fatty acid import protein CPT1b and the fatty acid oxidation enzyme LCAD in HFpEF mice following Adropin treatment.

FIG. 12 shows a schematic of Adropin function in HFpEF.

FIGS. 13A-13V show that metabolic dysfunction-associated fatty liver disease (MAFLD) coexists with heart failure preserved heart failure (HFpEF) in mice. FIG. 13A shows schematic of cohort 1 protocol. 12-wk old male C57BL/6J mice were maintained on either chow or HFpEF (HFD+L-NAME) dietary regimens with subsequent experimental interventions for up to 15-wks. FIGS. 13B-13H show analysis of experimental groups for body weight (FIG. 13B), body composition (FIG. 13C), glucose tolerance (FIGS. 13D and 13E), heart weight (FIG. 13F), liver weight (FIG. 13G), and lung weight (FIG. 13H). FIGS. 13I-13M shows representative images of M-mode and pulsed-wave (PW) doppler echocardiographic tracings (FIG. 13I) which evaluate LV mass (FIG. 13J), left ventricular ejection fraction (LVEF) (FIG. 13K), isovolumetric relaxation time (IVRT) (FIG. 13L) and left ventricular myocardial performance (LV MPI) functional parameters (FIG. 13M). FIGS. 13N-13R show representative images of liver hematoxylin and eosin (H&E) staining (FIG. 13N) which were used to assess steatosis (FIG. 13O), ballooning (FIG. 13P), inflammation (FIG. 13Q) and NAFLD/MAFLD activity score (NAS) (FIG. 13R). FIGS. 13S-13V show linear regression analyses performed to determine correlative relationships between NAS and glucose AUC (FIG. 13S), LVEF (FIG. 13T), IVRT (FIG. 13U) and LV MPI (FIG. 13V). N=8-14, *=P<0.05, **=P<0.01, ***=P<0.001, ****=P<0.0001.

FIGS. 14A-14L show transcriptomic and metabolomic profiles underlying cardio-hepatic interactions in HFpEF. FIGS. 14A-14F show bulk-RNA sequencing analysis of chow and HFpEF mice showing a heatmap of the top 30 differentially expressed genes (DEGs) in the heart (FIG. 14A) and liver (FIG. 14D). Visualization of DEGs by volcano plots in the heart (FIG. 14B) and liver (FIG. 14E) between the chow and HFpEF mice where genes with an adjusted P-value<0.05 and a log 2 fold change >1 are indicated by red dots and represent up-regulated genes. Genes with an adjusted P-value<0.05 and a log 2 fold change <−1 are indicated by blue dots and represent down-regulated genes. Gene ontology (GO) enrichment analyses identified the top 20 biological processes in the heart (FIG. 14C) and liver (FIG. 14F) and the shared processes between the two tissues are indicated by the colored arrows. FIGS. 14G-14I show Venn diagram (FIG. 14G) showing the total DEGs in the cardio-hepatic axis, highlighting three common genes including Ces2a (FIG. 14H), Cyp3a11 (FIG. 14I), and Elovl3 (FIG. 14J). N=4. FIGS. 14K-14L show untargeted metabolomics analysis of chow vs HFpEF heart (FIG. 14K) and liver (FIG. 14L) tissue samples using the mouse BioCyc database. Pathways with an enrichment factor >1 and −log 10 P value >0.5 are shown. N=4-8. **=P<0.01, ***=P<0.001, ****=P<0.0001.

FIGS. 15A-15T show that long-term adropin treatment attenuates cardiac dysfunction and structural remodeling in HFpEF. FIG. 15A shows illustration summarizing previous findings that adropin, a secreted hepatokine encoded by the energy-homeostasis (Enho) gene, restores cardiac glucose oxidation in diet-induced obesity. FIG. 15B shows schematic of cohort 2 protocol. 12-wk old male C57BL/6J mice were maintained on either chow or HFpEF (HFD+L-NAME) dietary regimens for 8 weeks and subject to daily intraperitoneal injections of either vehicle or recombinant adropin (Adr.) with subsequent experimental interventions for up to 12-wks of the study. FIGS. 15C-15D show daily and final body weight (after 28 days). FIGS. 15E-15F show intraperitoneal glucose tolerance test (IPGTT) time-course and area under the curve (AUC) analysis. FIGS. 15G-15K shows representative images of M-mode and pulsed-wave (PW) doppler echocardiographic tracings (FIG. 15G) which evaluate cardiac output (FIG. 15H), left ventricular ejection fraction (LVEF) (FIG. 15I), isovolumetric relaxation time (IVRT) (FIG. 15J) and left ventricular myocardial performance (LV MPI) (FIG. 15K) functional parameters. FIG. 15L shows representative images of masson's trichrome and WGA histological staining of the heart. FIGS. 15M-15O show normalized heart weight (ratio of heart weight to tibia length) (FIG. 15M) and quantification of histological staining to determine fibrosis (FIG. 15N) and cardiomyocyte size (FIG. 15O). FIGS. 15P-15T show representative images of liver hematoxylin and eosin (H&E) staining (FIG. 15P) used to assess steatosis (FIG. 15Q), ballooning (FIG. 15R), inflammation (FIG. 15S) and NAFLD/MAFLD activity score (NAS) (FIG. 15T). N=8-12. *=P<0.05, **=P<0.01, ***=P<0.001, ****=P<0.0001.

FIGS. 16A-16I show that adropin treatment inhibits flux into the hexosamine biosynthesis pathway (HBP) in HFpEF. FIG. 16A shows untargeted metabolomics analysis of cardiac HFpEF and HFpEF+Adr samples using the mouse BioCyc database. Pathways with an enrichment factor >1 and −log 10 P value >0.5 are shown. HBP=hexosamine biosynthesis pathway (HBP). FIGS. 16B-16H show representative immunoblots (FIG. 16B) and the respective quantification of total O-GlcNAcylation (FIG. 16C) and enzymes involved in HBP, fatty acid and glucose metabolism, specifically GFAT (FIG. 16D), OGT (FIG. 16E), OGA (FIG. 16F), CD36 (FIG. 16G), and PDK4 (FIG. 16H). FIG. 16I shows schematic of the HBP showing metabolomic changes in the experimental groups. Glucose enters the cell and undergoes a two-step conversion to fructose-6-phosphate, after which approximately 95% of it proceeds to glycolysis and 3-5% of it through HBP. GFAT catalyzes the first and rate-limiting step utilizing fructose-6-phosphate and glutamine that enters the cell as substrates to generate glucosamine-6-phosphate. Subsequent reactions are catalyzed by various enzymes using acetyl-CoA from fatty acid metabolism and UTP from nucleotide metabolism as substrates for de novo synthesis of UDP-GlcNAc, a metabolite used for protein O-GlcNAcylation catalyzed by OGT and OGA. At several steps of the pathway, adropin-mediated changes in substrate entry are shown. N=8-12, *=P<0.05, **=P<0.01, ***=P<0.001, ****=P<0.0001. Figure produced with license using Biorender software.

FIGS. 17A-17G show that adropin treatment inhibits FAO enzyme 0-GlcNAcylation to restore substrate metabolic flexibility in HFpEF. FIGS. 17A-17C show representative images of CPT1b and LCAD O-GlcNAcylation levels in chow, HFpEF, and HFpEF+Adr mice via 0-GlcNAcylation immunoprecipitation and western blotting (FIG. 17A) with their respective quantification (FIGS. 17B and 17C). FIGS. 17D-17F show enzymatic activity of cardiac LCAD (FIG. 17D). Linear regression analysis (FIG. 17E) showed a significant negative correlation between LCAD O-GlcNAcylation status and enzymatic activity from all experimental mouse groups that was further confirmed in vitro using recombinant LCAD (FIG. 17F). FIG. 17G shows summary of proposed mechanism of action of adropin in HFpEF. N=3-4, *=P<0.05, **=P<0.01, ***=P<0.001.

FIGS. 18A-18F show echocardiographic functional analyses of chow vs HFpEF mice in cohort 1 for E/A (FIG. 18A), E/e′ (FIG. 18B), IVCT (FIG. 18C), fractional shortening (FIG. 18D), SV (FIG. 18E), and left atrial area (FIG. 18F). N=8-14, **=P<0.01.

FIGS. 19A-19H show echocardiographic functional analyses of chow, HFpEF, and HFpEF+Adr. mice in cohort 2 for heart rate (FIG. 19A), MV E/E′ (FIG. 19B), MV E/A (FIG. 19C), fractional shortening (FIG. 19D), stroke volume (FIG. 19E), volume (systole) (FIG. 19F), volume (diastole) (FIG. 19G) and left atrial area (FIG. 19H). N=8-12.

FIGS. 20A-20Q show targeted metabolomic profiling of proteinogenic amino acids in the heart, specifically, threonine (FIG. 20A), alanine (FIG. 20B), lysine (FIG. 20C), proline (FIG. 20D), glycine (FIG. 20E), asparagine (FIG. 20F), serine (FIG. 20G), leucine (FIG. 20H), valine (FIG. 20I), arginine (FIG. 20J), methionine (FIG. 20K), histidine (FIG. 20L), phenylalanine (FIG. 20M), tyrosine (FIG. 20N), glutamine (FIG. 20O), aspartate (FIG. 20P), and glutamate (FIG. 20Q). N=8, *=P<0.05, **=P<0.01, ***=P<0.001.

FIGS. 21A-21Q show targeted metabolomic profiling of proteinogenic amino acids in the liver, specifically, threonine (FIG. 21A), alanine (FIG. 21B), lysine (FIG. 21C), proline (FIG. 21D), glycine (FIG. 21E), asparagine (FIG. 21F), serine (FIG. 21G), leucine (FIG. 21H), valine (FIG. 21I), arginine (FIG. 21J), methionine (FIG. 21K), histidine (FIG. 21L), phenylalanine (FIG. 21M), tyrosine (FIG. 21N), glutamine (FIG. 210), aspartate (FIG. 21P), and glutamate (FIG. 21Q). N=8, *=P<0.05, **=P<0.01, ***=P<0.001.

FIGS. 22A-22P show targeted metabolomic profiling of glycolysis, TCA cycle and energy metabolites in signature of the heart, specifically, hexose (FIG. 22A), glucose-6-phosphate (FIG. 22B), glyceraldehyde-3-phosphate (FIG. 22C), pyruvate (FIG. 22D), lactate (FIG. 22E), citrate (FIG. 22F), cis-aconitate (FIG. 22G), itaconate (FIG. 22H), AKG (FIG. 22I), succinate (FIG. 22J), fumarate (FIG. 22K), malate (FIG. 22L), carnitine (FIG. 22M), acetylcarnitine (FIG. 22N), creatine (FIG. 220), and creatinine (FIG. 22P). N=8, *=P<0.05, **=P<0.01, ***=P<0.001, ****=P<0.0001.

FIGS. 23A-23P show targeted metabolomic profiling of glycolysis, TCA cycle and energy metabolites in signature of the liver, specifically, hexose (FIG. 23A), glucose-6-phosphate (FIG. 23B), glyceraldehyde-3-phosphate (FIG. 23C), pyruvate (FIG. 23D), lactate (FIG. 23E), citrate (FIG. 23F), cis-aconitate (FIG. 23G), itaconate (FIG. 23H), AKG (FIG. 23I), succinate (FIG. 23J), fumarate (FIG. 23K), malate (FIG. 23L), carnitine (FIG. 23M), acetylcarnitine (FIG. 23N), creatine (FIG. 230), and creatinine (FIG. 23P). N=8, *=P<0.05, **=P<0.01, ***=P<0.001, ****=P<0.0001.

FIGS. 24A-24I show heart targeted metabolomics, specifically, hydroxyproline (FIG. 24A), methylhistidine (FIG. 24B), GABA (FIG. 24C), citrulline (FIG. 24D), ornithine (FIG. 24E), cytidine (FIG. 24F), hypoxanthine (FIG. 24G), adenosine (FIG. 24H), and taurine (FIG. 24I). N=8, *=P<0.05, **=P<0.01.

FIGS. 25A-25I show liver targeted metabolomics, specifically, hydroxyproline (FIG. 25A), methylhistidine (FIG. 25B), GABA (FIG. 25C), citrulline (FIG. 25D), ornithine (FIG. 25E), cytidine (FIG. 25F), hypoxanthine (FIG. 25G), adenosine (FIG. 25H), and taurine (FIG. 25I). N=8, *=P<0.05, **=P<0.01.

FIG. 26 shows gene ontology (GO) enrichment analysis of HFpEF vs HFpEF+Adr. mice. N=4.

FIG. 27 shows correlation between LCAD activity and LCAD enzyme abundance. Linear regression analysis revealed no correlation between LCAD activity and total LCAD protein. N=4 per group. Circles=chow mice, squares=HFpEF mice and triangles=HFpEF+Adr. mice.

FIGS. 28A-28B show protein abundance of PLIN5. FIG. 28A-28B show representative image of PLIN5 immunoblot (FIG. 28A) and quantification (FIG. 28B) of the PLIN5 enzyme expression in chow, HFpEF, and HFpEF+Adr. mice. N=8, **=P<0.01, ****=P<0.0001.

FIG. 29 shows analysis of differentially regulated genes for the heart of chow vs HFpEF mice.

FIG. 30 shows analysis of differentially regulated genes for the liver of chow vs HFpEF mice.

FIG. 31 shows pathway enrichment analysis for the heart of chow vs HFpEF mice.

FIG. 32 shows pathway enrichment analysis for the liver of chow vs HFpEF mice.

FIG. 33 shows pathway enrichment analysis for the heart of HFpEF mice vs HFpEF mice treated with Adropin.

FIG. 34 shows pathway enrichment analysis for the liver of HFpEF mice vs HFpEF mice treated with Adropin.

### DETAILED DESCRIPTION

The presently disclosed subject matter relates to the use of Adropin for the prevention and/or treatment of cardiometabolic disease, e.g., heart failure with preserved ejection fraction (HFpEF). In certain embodiments, Adropin is provided as recombinant protein. In certain embodiments, Adropin is provided using gene therapy methods for inducing endogenous production of Adropin. In certain embodiments, Adropin is endogenously produced in liver hepatocytes.

For purposes of clarity of disclosure and not by way of limitation, the detailed description is divided into the following subsections:
1. Definitions;
2. Models for Cardiometabolic Disease;
3. Adropin Therapy;
4. Pharmaceutical Compositions; and
5. Methods of Treatment

### 1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “culturing” refers to contacting a cell with a cell culture medium under conditions suitable to the survival, growth and/or proliferation of the cell.

The term “culture medium” refers to a nutrient solution used for growing cells, e.g., prokaryotic or eukaryotic cells, that typically provides at least one component from one or more of the following categories:
1) an energy source, usually in the form of a carbohydrate such as glucose;
2) all essential amino acids, and usually the basic set of twenty amino acids plus cysteine;
3) vitamins and/or other organic compounds required at low concentrations;
4) free fatty acids; and
5) trace elements, where trace elements are defined as inorganic compounds or naturally occurring elements that are typically required at very low concentrations, usually in the micromolar range.

The term “cell” refers to any suitable cell for use in the present disclosure, e.g., eukaryotic cells. For example, but not by way of limitation, suitable eukaryotic cells include animal cells, e.g., mammalian cells. In certain embodiments, suitable cells are cultured cells. In certain embodiments, suitable cells are host cells, recombinant cells, and recombinant host cells. In certain embodiments, suitable cells are cell lines obtained or derived from mammalian tissues which are able to grow and survive when placed in media containing appropriate nutrients and/or growth factors.

The terms “host cell,” “host cell line” and “host cell culture” are used interchangeably and refer to cells and their progeny into which exogenous nucleic acid can be subsequently introduced to create recombinant cells. In certain embodiments, these host cells can also be modified (i.e., engineered) to alter or delete the expression of certain endogenous host cell proteins. Host cells can include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein. The introduction of exogenous nucleic acid (e.g., by transfection) to these host cells would create recombinant cells that are derived from the original “host cell,” “host cell line” or “host cell line”. The terms “host cell,” “host cell line” and “host cell culture” can also refer to such recombinant cells and their progeny.

The terms “expression” or “expresses,” as used herein, refer to transcription and translation occurring within a cell, e.g., mammalian cell. In certain embodiments, the level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

The term “recombinant” when used with reference, e.g., to a cell, or nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. For example, recombinant cells express genes that are not found within the native (non-recombinant) form of the cell or express native genes that are otherwise abnormally expressed, under expressed, overexpressed or not expressed at all.

The terms “vector” or “plasmid”, which can be used interchangeably, as used herein, refer to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “nucleic acid molecule” and “nucleotide sequence,” as used herein, refers to a single or double-stranded covalently-linked sequence of nucleotides in which the 3′ and 5′ ends on each nucleotide are joined by phosphodiester bonds. The nucleic acid molecule can include deoxyribonucleotide bases or ribonucleotide bases, and can be manufactured synthetically in vitro or isolated from natural sources.

The terms “polypeptide,” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis or enzymatic synthesis. The terms can apply to amino acid polymers in which one or more amino acid residue is an artificial chemical mimetic of a corresponding naturally occurring amino acid, as well as to naturally occurring amino acid polymers and non-naturally occurring amino acid polymers.

The term “protein” is meant to refer to a sequence of amino acids for which the chain length is sufficient to produce the higher levels of tertiary and/or quaternary structure. This is to distinguish from “peptides” or other small molecular weight polypeptides that do not have such structure. In certain embodiments, the protein herein will have a molecular weight of at least about 15-20 kDa, e.g., about 20 kDa or greater. Examples of proteins encompassed within the definition herein include host cell proteins as well as all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “protein variant” or “polypeptide variant”, refers to a protein or polypeptide that comprise modifications and/or truncations compared to a parent or wild type protein or polypeptide. In certain embodiments, a protein variant can differ from the parent protein or wild type protein by at least one amino acid modification, e.g., from about one to about ten amino acid modifications. In certain embodiments, the sequence of a protein variant sequence has at least about 80%, at least about 90%, at least about 95% or at least about at least about 99% identity to a parent or wild type protein sequence. In certain embodiments, a protein variant can differ from another variant of the protein by at least one amino acid modification, e.g., from about one to about ten amino acid modifications. In certain embodiments, the sequence of a protein variant sequence has at least about 80%, at least about 90%, at least about 95% or at least about at least about 99% identity to a different variant of the protein.

As used herein, the term “mutation” refers to a mutation in an amino acid sequence or in a nucleic acid sequence. In certain embodiments, a mutation in an amino acid sequence can be a substitution (replacement), an insertion (addition), or a deletion (truncation) of at least one amino acid in the amino acid sequence. In certain embodiments, a mutation in a nucleic acid sequence can be a substitution (replacement), an insertion (addition), or a deletion (truncation) of at least nucleotide of the nucleic acid sequence.

The term “endogenous,” as used herein, refers to a nucleic acid molecule or polypeptide that is normally expressed in a cell or tissue.

The term “exogenous,” as used herein, refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “increase” is meant to alter positively by at least about 5%. An alteration can be an increase of about 5%, about 10%, about 25%, about 30%, about 50%, about 75%, about 100% or more.

By “reduce” is meant to alter negatively by at least about 5%. An alteration can be a decrease of about 5%, about 10%, about 25%, about 30%, about 50%, about 75% or more, even by about 100%.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “therapeutically effective amount” or “effective amount” refers to an amount of a virus composition that is sufficient to improve cardiac function, e.g., improve diastolic function, improve cardiac output, improve ejection fraction, improve isovolumetric relaxation time (IVRT), improve left ventricle myocardial performance index (LV MPI), improve cardiac morphology (e.g., left ventricle mass, volume, and/or wall thickness), reduces body weight, and/or reduce glucose intolerance. The amount of a virus composition that is therapeutically effective or effective may vary depending on the context. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more sign or symptoms, diminishment of extent of disease, stabilized (i.e., not worsening) state of disease, prevention of disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

“In combination with,” as used herein, means that a virus disclosed herein, and one or more agents are administered to a subject as part of a treatment regimen or plan.

### 2. Models for Cardiometabolic Disease

The mouse HFpEF model used in this study was first reported in Schiattarella et al. (2019), Nature 568: 351-356 (PMID: 30971818). Adult C57BL/6N or C57BL/6J mice (i.e. C57BL/6 wildtype) are placed on a diet of either regular chow (“control”), or a high fat diet (60% fat) supplemented with 0.5 g/L L-NAME in drinking water (“HFpEF”) for 5-20 weeks.

### 3. Adropin Therapy

The Adropin-GPR19 pathway represents a novel therapeutic target, consisting of an endogenously produced ligand (Adropin), and a membrane-bound receptor (GPR19). Previous work from our group has shown that: (i) Adropin treatment restores glucose oxidation in the hearts of diabetic mice; (ii) Adropin treatment decreases liver glucose production in diabetic mice; and (iii) that Adropin function in cardiac cells is dependent on GPR19.

The present disclosure shows that: (i) Adropin treatment restores diastolic function in vivo in a mouse model of heart failure with preserved ejection fraction (HFpEF)—a model which combines obesity/diabetes and hypertension, two cardiometabolic disease features; and that (ii) Adropin treatment reduces body weight and glucose intolerance in the same HFpEF model. Thus, Adropin-GPR19 pathway is a novel, targetable pathway in the treatment of metabolic diseases. Non-limiting examples of metabolic diseases include HFpEF, type 2 diabetes, hypertension, and non-alcoholic fatty liver disease (NAFLD).

Full-length Adropin is endogenously produced in liver hepatocytes, and a cleaved bioactive version of the peptide (Adropin³⁴⁻⁷⁶) is secreted into the systemic circulation to act on other peripheral tissues.

### 3.1 Recombinant Polypeptide

Recombinant Adropin polypeptide can be produced using methods known in the art. Recombinant Adropin polypeptide can be produced as the full-length peptide, or as the cleaved product of amino acids 34-76.

In certain embodiments, the polypeptide encodes a human Adropin or a functional fragment thereof. In certain embodiments, the human Adropin has an amino acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous or identical to the amino acid sequence set forth in GenBank/NCBI database accession no. NP_940975.2. In certain embodiments, the Adropin polypeptide can contain substitutions, insertions, or deletions relative to the amino acid sequence set forth in GenBank/NCBI database accession no. NP_940975.2, that do not significantly alter the function or activity of the human Adropin.

In certain embodiments, the human Adropin polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 1, which is provided below.

(SEQ ID NO.: 1)
MGAAISQGALIAIVCNGLVGFLLLLLWVILCWACHSRSADVDS
LSESSPNSSPGPCPEKAPPPQKPSHEGSYLLQP

In certain embodiments, the human Adropin polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 2, which is provided below.

(SEQ ID NO.: 2)
CHSRSADVDSLSESSPNSSPGPCPEKAPPPQKPSHEGSYLLQP

Constructs encoding Adropin, or constructs encoding related protein variants, as described herein, can be introduced into cells as one or more DNA molecules or constructs, in many cases in association with one or more markers to allow for selection of host cells which contain the construct(s). The constructs can be prepared in conventional ways, where the coding sequences and regulatory regions can be isolated, as appropriate, ligated, cloned in an appropriate cloning host, analyzed by restriction or sequencing, or other convenient means. Particularly, using PCR, individual fragments including all or portions of a functional unit can be isolated, where one or more mutations can be introduced using “primer repair”, ligation, in vitro mutagenesis, etc. as appropriate.

In certain embodiments, the expression construct encoding the polypeptide or protein of interest is integrated into one or more expression vectors. In certain embodiments, the expression vector is a nucleic acid and provides all required elements for the amplification of said vector in a mammalian cell. In certain embodiments, an expression vector is a vehicle for the introduction of an expression construct into a modified mammalian cell according to the subject matter of the present disclosure. In certain embodiments, a construct can be introduced as a single DNA molecule encoding multiple genes, or different DNA molecules having one or more genes. In certain embodiments, multiple constructs can be introduced simultaneously or consecutively, each with the same or different DNA molecule.

The construct(s) once completed and demonstrated to have the appropriate sequences can then be introduced into a host cell by any convenient means. The constructs can be integrated and packaged into non-replicating, defective viral genomes like Adenovirus, Adeno-associated virus (AAV), or Herpes simplex virus (HSV) or others, including retroviral vectors, for infection or transduction into cells. In certain embodiments, the constructs can include viral sequences for transfection, if desired. Alternatively, the construct can be introduced by fusion, electroporation, biolistics, transfection, lipofection, or the like. The host cells will in some cases be grown and expanded in culture before introduction of the construct(s), followed by the appropriate treatment for introduction of the construct(s) and integration of the construct(s). The cells will then be expanded and screened by virtue of a marker present in the construct.

In certain embodiments, expressing one or more recombinant proteins of interest in a host cell includes culturing a cell comprising one or more nucleic acid(s) encoding the polypeptide or protein of interest, under conditions suitable for expression of the polypeptide or protein. Non-limiting examples of such cells are disclosed herein, e.g., mammalian cells can be used to express the polypeptide or protein. In certain embodiments, a host cell is transfected with a vector containing the nucleic acid sequence suitable for expression of said polypeptide or protein of interest.

### 3.2 Gene Therapy Methods

Recombinant Adropin provides a robust response in vivo, resulting in a significant increase in glucose tolerance and diastolic function in a mouse model of HFpEF. However, the in vivo stability of recombinant Adropin is sub-optimal for therapeutic use, with 96% of the peptide cleared from circulation within 1 hour. The present disclosure relates to a gene therapy approach for providing more consistent therapeutic outcomes. The ENHO gene (which produces Adropin) is encoded in an adenoviral-associated viral (AAV) vector, a standard, non-integrating virus that is routinely used for gene therapy in humans. In certain embodiments, the virus isotype is AAV1, AAV2, AAV3, AAV6, AAV6.2, AAV7, AAV8, AAV9, AAVrh10, AAV/DJ, or AAV/DJ8.

In certain embodiments, the ENHO gene is expressed under the control of a hepatocyte promoter to limit expression in non-hepatic tissues. Targeting the ENHO virus to the liver is beneficial for several reasons. For example, as the site of the majority of endogenous Adropin production, the liver contains the necessary packaging and secretion mechanisms to deliver Adropin to the circulation. In addition, the liver contains 10-15% of the body blood volume, making it an ideal site of circulating protein production. Finally, the half-life of differentiated adult hepatocytes is 200-300 days, meaning that individual treatments are likely to maintain Adropin protein production for 3-6+ months.

In certain embodiments, the virus disclosed herein includes a nucleic acid molecule encoding ENHO or a functional fragment thereof. In certain embodiments, the nucleic acid molecule encodes human ENHO or a functional fragment thereof. In certain embodiments, the human ENHO has a nucleic acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous or identical to the nucleic acid sequence set forth in GenBank/NCBI database accession no. NM_198573.3. In certain embodiments, the nucleic acid molecule encoding human ENHO can contain substitutions (e.g., conservative substitutions), insertions, or deletions relative to the nucleic acid sequence set forth in GenBank/NCBI database accession no. NM_198573.3, that do not significantly alter the function or activity of the human Adropin.

In certain embodiments, the nucleic acid molecule encoding human ENHO comprises or consists of a nucleic acid sequence that is at least about 85%, about 90%, about 95%, about 96%, about 97%, about 98%, about 99% or 100% homologous or identical to the nucleic acid sequence set forth in SEQ ID NO: 3, which is provided below.

(SEQ ID NO.: 3)
AGACCGCCCGCGGCAGAGGCCGCCCCGGTCGCGCCGCGGCGGG
AGCGGCCGGTGGAGGCTGCGCGGCCGAGGGGGAGGGCCGGGGG
AGCGGACGTCGCCTCTGCTGGTCTCCCACCTCCCGCCGCCCCC
CGCCCGCAGGCTCCCAAGCCTTAGTCGGCGCCGAGCATCCCGC
TGCCCCGGACCCTCCCGCGGGCGCGCACCAGGCTCAACTCAGG
CTCAGGACTGCAGGTAGACATCTCCACTGCCCAGGAATCACTG
AGCGTGCAGACAGCACAGCCTCCTCTGAAGGCCGGCCATACCA
GAGTCCTGCCTCGGCATGGGCCTCACCATTGAGGCAGCTCCAC
TGTCTGTGCTGGTCTGAGGGTGCTGCCTGTCATGGGGGCAGCC
ATCTCCCAGGGGGCCCTCATCGCCATCGTCTGCAACGGTCTCG
TGGGCTTCTTGCTGCTGCTGCTCTGGGTCATCCTCTGCTGGGC
CTGCCATTCTCGCTCTGCCGACGTTGACTCTCTCTCTGAATCC
AGTCCCAACTCCAGCCCTGGCCCCTGTCCTGAGAAGGCCCCAC
CACCCCAGAAGCCCAGCCATGAAGGCAGCTACCTGCTGCAGCC
CTGAAGGCCCCTGGCCTAGCCTGGAGCCCAGGACCTAAGTCCA
CCTCACCTAGAGCCTGGAATTAGGATCCCAGAGTTCAGCCAGC
CTGGGGTCCAGAACTCAAGAGTCCGCCTGCTTGGAGCTGGACC
CAGCGGCCCAGAGTCTAGCCAGCTTGGCTCCAATAGGAGCTCA
GTGGCCCTAAGGAGATGGGCCTGGGGTGGGGGCTTATGAGTTG
GTGCTAGAGCCAGGGCCATCTGGACTATGCTCCATCCCAAGGG
CCAAGGGTCAGGGGCCGGGTCCACTCTTTCCCTAGGCTGAGCA
CCTCTAGGCCCTCTAGGCTGGGGAAGCAAACTGGAACCCATGG
CAATAATAGGAGGGTGTCCAGGCTGGGCCCCTCCCCTGGTCCT
CCCAGTGTTTGCTGGATAATAAATGGAACTATGGCTCTA

In certain embodiments, the nucleic acid molecule encoding ENHO is integrated into the genome of the virus, where the expression of the nucleic acid molecule is operably linked to a promoter that is active or activatable in the virus infected cell. As used herein, “operably linked” means that a promoter is in a correct functional location and/or orientation in relation to a nucleic acid locus to control transcriptional initiation and/or expression of that locus. In certain embodiments, the nucleic acid encoding ENHO is operably linked to a promoter which induces expression in hepatocytes. Non-limiting examples of promoters include thyroxine binding globulin (TBG), albumin (ALB), hepatitis virus (HBV), alpha-1 antitrypsin (AAT), and human cytomegalovirus (CMV).

### 4. Pharmaceutical Compositions

The present disclosure further provides pharmaceutical compositions that include a virus that expresses Adropin or a functional fragment thereof. For example, but not by way of limitation, the methods can include administering to the subject a virus that comprises a nucleic acid that encodes Adropin, e.g., human Adropin, or a functional fragment thereof. In certain embodiments, the pharmaceutical composition includes an effective amount of the virus. In certain embodiments, the pharmaceutical composition can be prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof.

In certain embodiments, the pharmaceutical composition described herein further includes a pharmaceutically acceptable carrier, e.g., an excipient. In certain embodiments, the pharmaceutically acceptable carrier includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically acceptable carriers include gels, bioadsorbable matrix materials, implantation elements containing the virus, and any other suitable vehicle, delivery, or dispensing means or material.

In certain embodiments, the pharmaceutically acceptable carrier can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminium hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide other calcium salts, and combinations thereof. In certain embodiments, the virus disclosed herein can be propagated in suitable host cells, isolated from host cells, and stored in conditions that promotes stability and integrity of the virus, such that loss of infectivity over time is minimized. In certain embodiments, the virus disclosed herein can be stored by freezing or drying, such as by lyophilization. In certain embodiments, prior to administration, the stored virus can be reconstituted (if dried for storage) and diluted in a pharmaceutically acceptable carrier for administration.

In certain embodiments, the pharmaceutical compositions disclosed herein can be provided systemically. In certain embodiments, the presently disclosed viruses or pharmaceutical compositions are directly injected into an organ of interest (e.g., liver). Alternatively, the presently disclosed viruses or pharmaceutical compositions are provided indirectly to the organ of interest, for example, by administration into the circulatory system. The viruses or pharmaceutical compositions can be provided intravenously, intraperitoneally, or subcutaneously. When administering a therapeutic composition of the presently disclosed subject matter (e.g., a pharmaceutical composition comprising a presently disclosed virus), it can be formulated in a unit dosage injectable form (solution, suspension, emulsion).

In certain embodiments, the pharmaceutical composition comprises a therapeutically effective amount of an Adropin-based therapy. In certain embodiments, the pharmaceutical composition further comprises a pharmaceutically acceptable carrier. In certain embodiments, the Adropin-based therapy is an AAV, wherein the AAV comprises a heterologous nucleic acid encoding an ENHO gene. In certain embodiments, the ENHO gene comprises SEQ ID NO.: 3. In certain embodiments, the Adropin-based therapy is a recombinant Adropin peptide. In certain embodiments, the recombinant Adropin peptide comprises SEQ ID NO.: 1. In certain embodiments, the recombinant Adropin peptide comprises SEQ ID NO.: 2. In certain embodiments, the pharmaceutical composition is administered intravenously, intraperitoneally, or subcutaneously.

### 5. Methods of Treatment

The present disclosure provides methods of treating a subject having a cardiometabolic disease. In certain embodiments, the methods include administering to the subject a virus that expresses Adropin or a functional fragment thereof. For example, but not by way of limitation, the methods can include administering to the subject a virus that comprises a nucleic acid that encodes Adropin, e.g., human Adropin, or a functional fragment thereof. In certain embodiments, the methods include administering to the subject recombinant Adropin peptide. In certain embodiments, the abundance (circulating levels) of Adropin is increased. In certain embodiments, the Adropin-based therapy is administered intravenously, intraperitoneally, or subcutaneously.

In certain embodiments, recombinant Adropin peptide is administered in an amount between about 1 to about 200 mg, between about 1 to about 100 mg, between about 10 to about 50 mg, about 10 mg, about 20 mg, about 25 mg, about 40 mg, about 50 mg, about 75 mg, or about 100 mg. In certain embodiments, recombinant Adropin peptide is administered in an amount between about 10 to about 1000 ng, between 100 to about 1000 ng, between about 500 to about 1000 ng, about 100 ng, about 125 ng, about 150 ng, about 200 ng, about 300 ng, about 400 ng, about 500 ng, about 750 ng, or about 1000 ng. In certain embodiments, recombinant Adropin peptide is administered in an amount that is at least about 100 ng, at least about 125 ng, at least about 150 ng, at least about 200 ng, at least about 300 ng, at least about 400 ng, at least about 500 ng, at least about 750 ng, at least about 1000 ng, at least about 50 mg, at least about 10 mg, at least about 20 mg, at least about 25 mg, at least about 40 mg, at least about 50 mg, at least about 75 mg, or at least about 100 mg. In certain embodiments, recombinant Adropin peptide is administered in an amount between about 1 ng/kg to about 200 mg/kg. In certain embodiments, recombinant Adropin peptide is administered in an amount between about 1 to about 200 mg/kg, between 1 to about 100 mg/kg, between about 1 to about 10 mg/kg, between about 10 to about 50 mg/kg, about 1 mg/kg, about 2 mg/kg, about 5 mg/kg, about 10 mg/kg, about 20 mg/kg, about 25 mg/kg, about 40 mg/kg, about 50 mg/kg, about 75 mg/kg, about 100 mg/kg, or about 200 mg/kg. In certain embodiments, recombinant Adropin peptide is administered in an amount between about 1 to about 1000 ng/kg, between 1 to about 100 ng/kg, between about 1 to about 10 ng/kg, between about 10 to about 50 ng/kg, between about 50 to about 100 ng/kg, between about 100 to about 500 ng/kg, between about 500 to about 1000 ng/kg, about 1 ng/kg, about 2 ng/kg, about 5 ng/kg, about 10 ng/kg, about 20 ng/kg, about 25 ng/kg, about 40 ng/kg, about 50 ng/kg, about 75 ng/kg, about 100 ng/kg, about 200 ng/kg, about 300 ng/kg, about 400 ng/kg, about 500 ng/kg, about 600 ng/kg, about 700 ng/kg, about 800 ng/kg, or about 900 ng/kg. In certain embodiments, recombinant Adropin peptide is administered in an amount that is at least about 1 ng/kg, at least about 2 ng/kg, at least about 5 ng/kg, at least about 10 ng/kg, at least about 20 ng/kg, at least about 25 ng/kg, at least about 40 ng/kg, at least about 50 ng/kg, at least about 75 ng/kg, at least about 100 ng/kg, at least about 200 ng/kg, at least about 300 ng/kg, at least about 400 ng/kg, at least about 500 ng/kg, at least about 600 ng/kg, at least about 700 ng/kg, at least about 800 ng/kg, at least about 900 ng/kg, at least about 1 mg/kg, at least about 2 mg/kg, at least about 5 mg/kg, at least about 10 mg/kg, at least about 20 mg/kg, at least about 25 mg/kg, at least about 40 mg/kg, at least about 50 mg/kg, at least about 75 mg/kg, at least about 100 mg/kg, or at least about 200 mg/kg.

In certain embodiments, recombinant Adropin peptide is administered from about 1 week to about 4 weeks, about 1 week to about 2 weeks, about 2 weeks to about 4 weeks, about 1 week, about 2 weeks, about 3 weeks, or about 4 weeks. In some embodiments, the composition is administered daily. In some embodiments, the composition is administered once a week, twice a week, three times a week, or four times a week. In certain embodiments, the Adropin peptide is administered intravenously, intraperitoneally, or subcutaneously.

In certain embodiments, the Adropin-based therapy is a composition comprising a virus, wherein the virus comprises a heterologous nucleic acid encoding an Energy Homeostasis-associated (ENHO) gene. In certain embodiments, the virus is administered into the liver of the subject. In certain embodiments, the virus is administered to hepatocytes of the subject. In certain embodiments, the virus is an adenoviral associated virus (AAV). In certain embodiments, the AAV isotype is selected from the group consisting of AAV1, AAV2, AAV3, AAV6, AAV6.2, AAV7, AAV8, AAV9, AAVrh10, AAV/DJ, and AAV/DJ8. In certain embodiments, the virus is administered intravenously, intraperitoneally, or subcutaneously.

In certain embodiments, the virus is administered in an amount between about 10⁵ to about 10²⁰ genome copies per kg, between about 10⁵ to about 10¹⁰ genome copies per kg, between about 10¹⁰ to about 10¹⁵ genome copies per kg, between about 10¹⁵ to about 10²⁰ genome copies per kg, about 10⁵ genome copies per kg, about 10⁶ genome copies per kg, about 10⁷ genome copies per kg, about 10⁸ genome copies per kg, about 10⁹ genome copies per kg, about 10¹⁰ genome copies per kg, about 10¹¹ genome copies per kg, about 10¹² genome copies per kg, about 10¹³ genome copies per kg, about 10¹⁴ genome copies per kg, about 10¹⁵ genome copies per kg, about 10¹⁶ genome copies per kg, about 10¹⁷ genome copies per kg, about 10¹⁸ genome copies per kg, about 10¹⁹ genome copies per kg, or about 10²⁰ genome copies per kg. In certain embodiments, the virus is administered in an amount that is at least about 10⁵ genome copies per kg, at least about 10⁶ genome copies per kg, at least about 10⁷ genome copies per kg, at least about 10⁸ genome copies per kg, at least about 10⁹ genome copies per kg, at least about 10¹⁰ genome copies per kg, at least about 10¹¹ genome copies per kg, at least about 10¹² genome copies per kg, at least about 10¹³ genome copies per kg, at least about 10¹⁴ genome copies per kg, at least about 10¹⁵ genome copies per kg, at least about 10¹⁶ genome copies per kg, at least about 10¹⁷ genome copies per kg, at least about 10¹⁸ genome copies per kg, at least about 10¹⁹ genome copies per kg, or at least about 10²⁰ genome copies per kg.

In certain embodiments, the cardiometabolic disease is selected from the group consisting of heart failure with preserved ejection fraction (HFpEF), type-2 diabetes, hypertension, or non-alcoholic fatty liver disease (NAFLD).

In certain embodiments, the method disclosed herein improves diastolic function, improves cardiac output, improves ejection fraction, improves isovolumetric relaxation time (IVRT), improves left ventricle myocardial performance index (LV MPI), improves cardiac morphology (e.g., left ventricle mass, volume, and/or wall thickness), reduces body weight, and/or reduces glucose intolerance. In certain embodiments, the method disclosed herein improves one or more feature selected from the group consisting of diastolic function, cardiac output, ejection fraction, IVRT, LV MPI, cardiac morphology (e.g., left ventricle mass, volume, and/or wall thickness), body weight, glucose intolerance, and combinations thereof. In certain embodiments, the method disclosed herein improves one or more features about 1 week post-treatment, about 2 weeks post-treatment, about 3-weeks post-treatment, about 4 weeks post-treatment, about 5 weeks post-treatment, about 6 weeks post-treatment, about 7 weeks post-treatment, about 8 weeks post-treatment, about 9 weeks post-treatment, or about 10 weeks post-treatment.

### EXAMPLES

The present disclosure will be better understood by reference to the following Examples, which are provided as exemplary of the presently disclosed subject matter, and not by way of limitation.

### Example 1: Recombinant Adropin Therapy

The present example demonstrates use of recombinant Adropin in a model of cardiometabolic disease. Models for HFpEF will be administered recombinant Adropin.

### Methods

Administration of Recombinant Adropin. Mice received low-fat chow or HFpEF diet for 10 weeks. For the following 4 weeks, the mice were maintained on respective diets and received daily injections of recombinant Adropin. Body weight was measured every day during the four weeks of treatment.

Cardiac function testing. After 2 weeks, mice were tested using ultrasonography to measure cardiac function and morphology. To determine systolic function and morphology, mice were tested using M-mode echocardiography to measure left ventricle (LV) mass, volume, wall thickness, and contraction (LV ejection fraction and fractional shortening). To determine diastolic function, mice were tested using Doppler to measure LV relaxation and filling (E/A ratio, E/e′ ratio, and isovolumetric relaxation time [IVRT]).

Glucose tolerance testing. Glucose tolerance was tested (IPGTT), following a 2 g/kg bolus of glucose. Blood was collected at 15 mins, 30 mins, 45 mins, 60 mins, 90 mins, and 120 mins, and measured using a standard glucometer (Bayer Next EZ). Time-courses for blood glucose uptake will be generated and analyzed for significance using standard statistical tests.

### Results

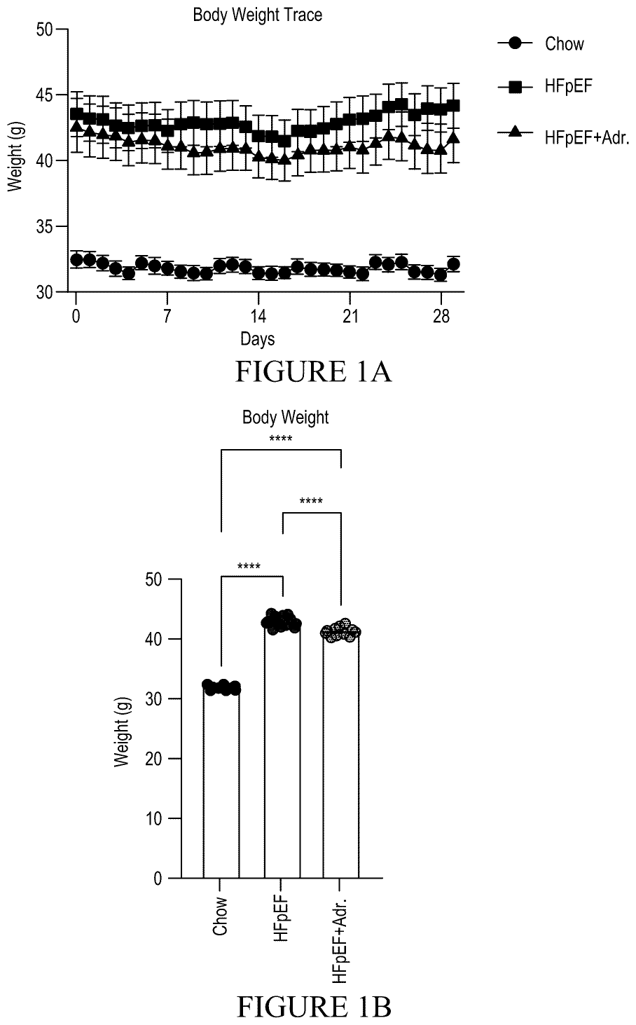

Body weight reduction. Body weight was recorded for four weeks while Adropin was administered. Beginning on day 8, body weight was reduced for HFpEF ENHO mice which received Adropin versus HFpEF mice which did not receive Adropin. The difference in body weight increased over the duration of Adropin treatment (FIG. 1A).

Relative body weight was calculated to show changes in the relative increase or decrease in body weight per mouse in each group between days 0 and 29. The relative body weight of mice on HFpEF diet (without Adropin) was significantly greater than mice on control chow diet (p-value<0.0001) (FIG. 1B). The relative body weight of HFpEF mice that received Adropin was significantly lower than HFpEF mice without treatment (p-value<0.0001) and significantly greater than mice on control chow diet (p-value<0.0001).

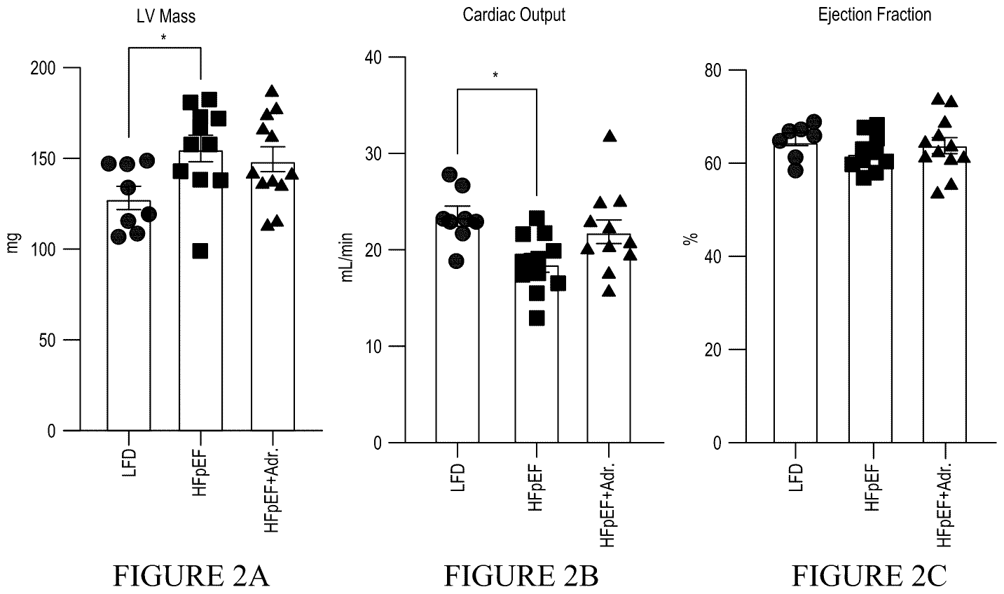

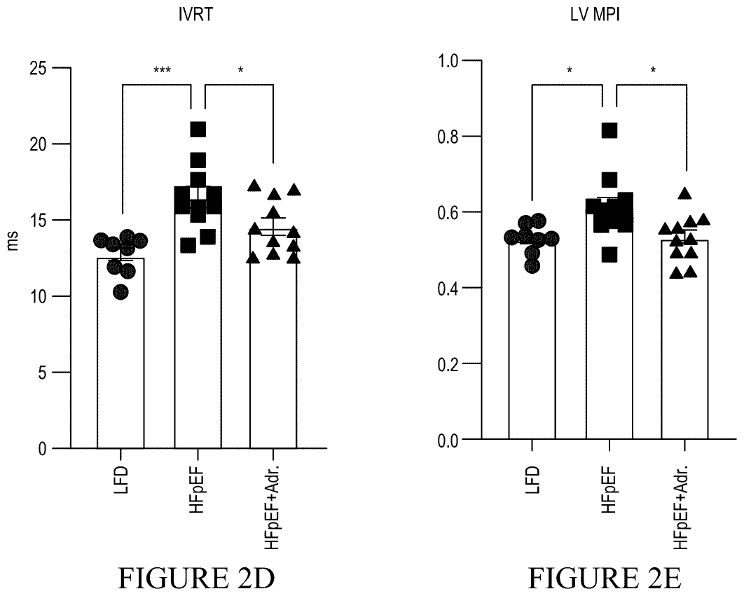

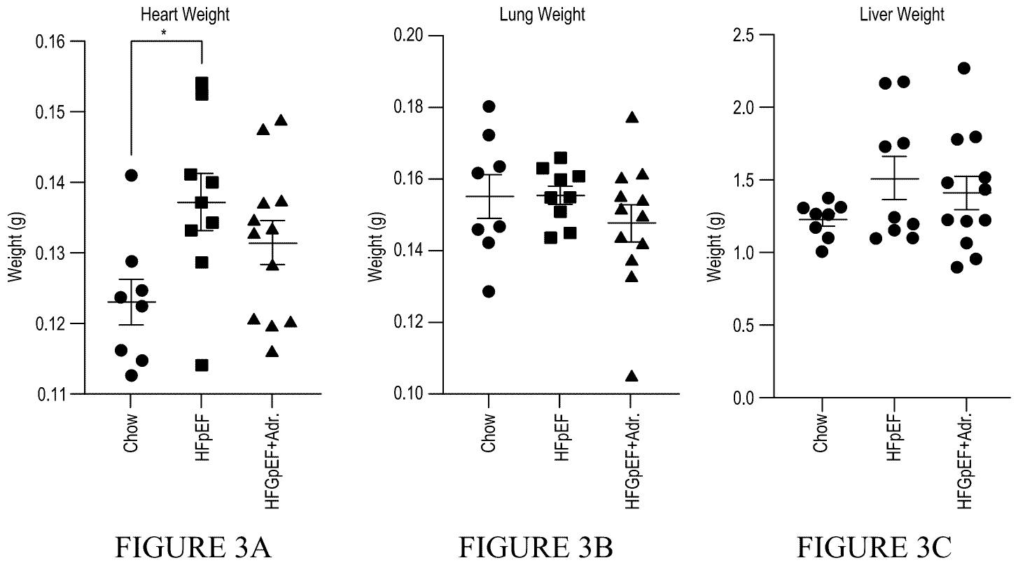

Measurement of Cardiac Function. Following 2 weeks of Adropin treatment, mice were evaluated for left ventricle (LV) mass, cardiac output, ejection fraction, isovolumetric relaxation time (IVRT), and left ventricle myocardial performance index (LV MPI). LV mass was significantly greater in HFpEF versus control chow mice (p-value <0.05) (FIG. 2A). Average LV mass was lower in HFpEF mice which received Adropin versus HFpEF mice without treatment. Similar trends were observed following 4 weeks of Adropin treatment: LV mass was significantly greater in HFpEF versus control chow mice (p-value <0.05) (FIG. 3A); average LV mass was lower in HFpEF mice which received Adropin versus HFpEF mice without treatment. After 4 weeks, no differences were observed in lung or liver mass (FIG. 3B-3C).

Cardiac output was significantly lower in HFpEF mice versus control chow mice (p-value <0.05) (FIG. 2B). Average cardiac output was greater in HFpEF mice which received Adropin versus HFpEF mice without treatment.

Ejection fraction was lower on average in HFpEF mice versus control chow mice (FIG. 2C). Average ejection fraction was greater in HFpEF mice which received Adropin versus HFpEF mice without treatment.

IVRT was significantly greater in HFpEF mice versus control chow mice (p-value <0.001) (FIG. 2D). IVRT was significantly lower in HFpEF which received Adropin versus HFpEF mice without treatment (p-value <0.05).

LV MPI was significantly greater in HFpEF mice versus control chow mice (p-value <0.05) (FIG. 2E). LV MPI was significantly lower in HFpEF which received Adropin versus HFpEF mice without treatment (p-value <0.05).

### Measurement of Adropin Abundance and Blood Glucose.

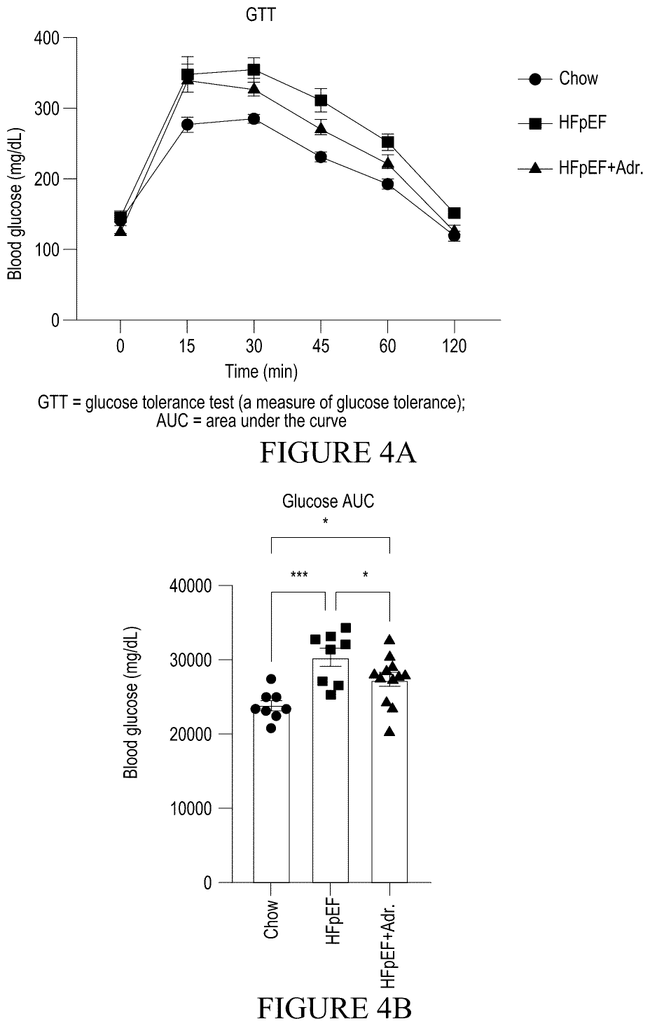

Average blood glucose was greater in HFpEF mice without Adropin versus control chow mice at all timepoints (FIG. 4A). In HFpEF mice that received Adropin, average blood glucose was similar to HFpEF mice without treatment at the 15 minute timepoint, but gradually decreased to match control chow mice by the 120 minute timepoint. During AUC analysis, blood glucose was significantly greater in HFpEF mice without Adropin versus control mice (p-value <0.001) (FIG. 4B) and also significantly greater than HFpEF mice that received Adropin (p-value <0.05). Blood glucose was additionally significantly greater in HFpEF mice that received Adropin than control chow mice (p-value <0.05).

### Adropin-Mediated Restoration of Diastolic Function is GPR19-Dependent

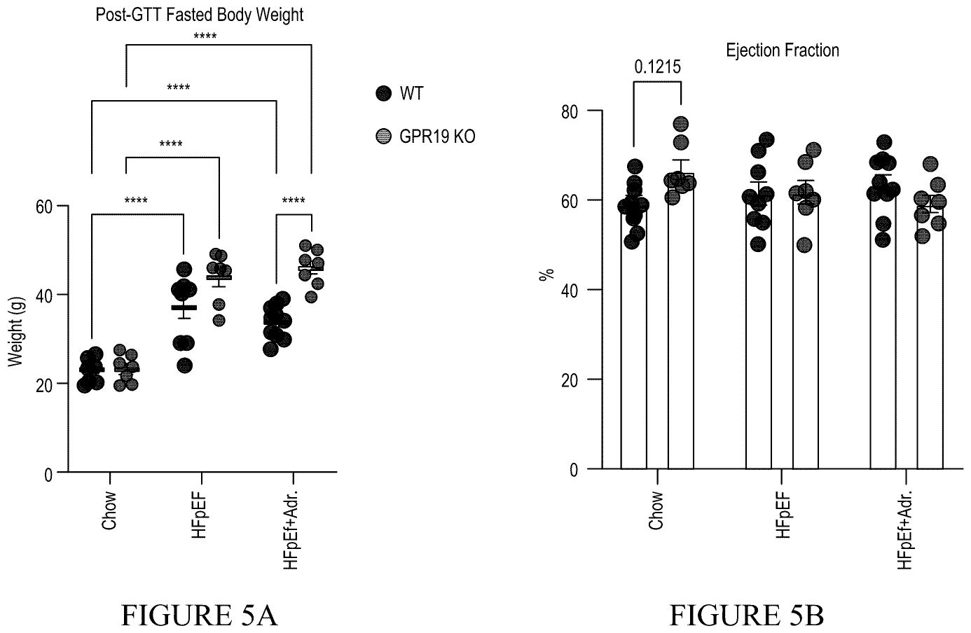

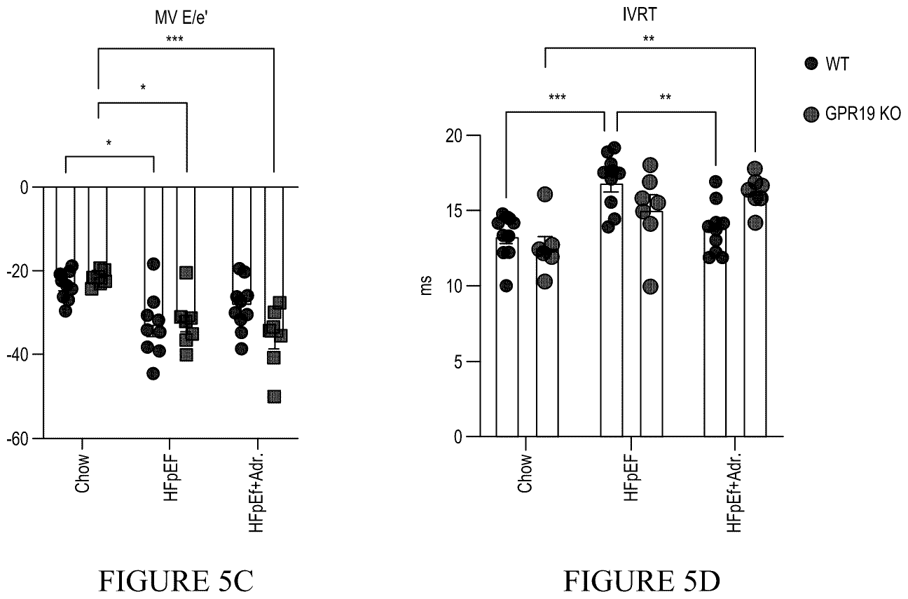

Wildtype and GPR19-knockout (GPR19-KO) mice were given normal chow (control mice), HFpEF diet without Adropin, or HFpEF with Adropin. In both wildtype and GPR19-KO mice, fasted body weight was significantly greater in HFpEF mice versus control mice (p-value <0.0001) and HFpEF mice that received Adropin (p-value <0.0001) (FIG. 5A). In addition, the body weight of GPR19-KO HFpEF mice that received Adropin was significantly greater than body weight of wildtype HFpEF mice that received Adropin (p-value <0.0001).

Ejection fraction was lower on average in wildtype mice versus GPR19-KO mice (p-value=0.1215) (FIG. 5B).

Diastolic function (MV E/e′) was significantly lower in wildtype HFpEF mice versus wildtype control mice (p-value<0.05) and increased on average in wildtype HFpEF mice that received Adropin versus wildtype HFpEF mice without Adropin. Diastolic function of wildtype HFpEF mice that received Adropin was not significantly different from wildtype control mice. Diastolic function was significantly lower in GPR19-KO HFpEF mice versus GPR19-KO control mice (p-value<0.05). In contrast to wildtype mice, diastolic function remained significantly lower in GPR19-KO mice versus control mice (p-value <0.001).

IVRT was significantly greater in wildtype HFpEF mice versus wildtype control mice (p-value <0.001), and significantly lower in wildtype HFpEF which received Adropin versus wildtype HFpEF mice without treatment (p-value <0.01) (FIG. 5D). IVRT of wildtype HFpEF mice that received Adropin was not significantly different from wildtype control mice. IVRT was greater in GPR19-KO HFpEF mice versus GPR19-KO control mice. In contrast to wildtype mice, IVRT was significantly greater in GPR19-KO HFpEF mice versus GPR19-KO control mice (p-value <0.01).

### Discussion

Therapy using recombinant Adropin improved diastolic function, as evidenced through IVRT, LV MPI, relative body weight, and reduced glucose intolerance. Improvements were additionally observed in LV mass and cardiac output. These results were observed in a mouse model of HFpEF which combines cardiometabolic disease features of obesity/diabetes and hypertension.

The improvements to diastolic function in Adropin-treated HFpEF mice, were not observed in GPR19-KO HFpEF mice. Such observations were made regarding fasted body weight, MV E/e′, and IVRT. These observations show that Adropin treatment is mediated through the GPR19 receptor. Thus, the Adropin-GPR19 pathway is a therapeutic target for cardiometabolic diseases, such as HFpEF.

### Example 2: ENHO Gene Therapy

The present example demonstrates use of the gene therapy method for providing Adropin in a model of cardiometabolic disease. Models for HFpEF will be administered AAV8-TBG-ENHO or control virus. Adropin gene therapy is expected to restore diastolic function, reduce body weight, and reduce glucose intolerance.

### Methods

Administration of Gene Therapy. After 10 weeks of chow or HFpEF diet, mice will receive a single tail vein injection of either AAV8-TBG-GFP (control) or AAV8-TBG-ENHO (Adropin) at 1×10¹² genome copies/kg. Mice will be maintained on the same diet after injection for 4 weeks. Adropin abundance in plasma will be measured by ELISA (Phoenix Peptides ELISA kit).

Cardiac function testing. After 4 weeks, mice will undergo ultrasonography to measure cardiac function and morphology. To determine systolic function and morphology, mice will undergo M-mode echocardiography to measure left ventricle (LV) mass, volume, wall thickness, and contraction (LV ejection fraction and fractional shortening). To determine diastolic function, mice will undergo Doppler to measure LV relaxation and filling (E/A ratio, E/e′ ratio, and isovolumetric relaxation time [IVRT]).

Glucose tolerance testing. Mice will undergo intraperitoneal glucose tolerance testing (IPGTT), following a 2 g/kg bolus of glucose. Blood will be collected at 15 mins, 30 mins, 45 mins, 60 mins, 90 mins, and 120 mins, and measured using a standard glucometer (Bayer Next EZ). Time-courses for blood glucose uptake will be generated and analyzed for significance using standard statistical tests.

### Results

Measurement of Cardiac Function. Diastolic function is expected to show significant improvement (E/A, E/e′, and/or IVRT) in HFpEF ENHO mice relative to chow and HFpEF mice given the control virus. Cardiac morphology (LV mass, volume, and/or wall thickness) can also be significantly improved in HFpEF ENHO mice relative to chow and HFpEF mice given the control virus.

Measurement of Adropin Abundance and Blood Glucose. Adropin abundance (circulating levels) is expected to be significantly increased in ENHO virus HFpEF mice relative to chow mice and HFpEF mice given control virus. Blood glucose is expected to be significantly decreased in ENHO virus HFpEF mice relative to chow mice and HFpEF mice given control virus.

### Example 3: Adropin Restores Function in Heart Failure with Preserved Ejection Fraction by Reversing Excessive O-GlcNAcylation

Diabetic cardiomyopathy (DCM) is a major complication of diabetes, and has been recognized as a cause of heart failure independent of other common risk factors. Metabolic inflexibility is a hallmark feature, where increased free fatty acid availability and decreased myocardial glucose uptake lead to an over-reliance on fatty acid oxidation, reducing cardiac work efficiency. Energetic inefficiency in diabetic hearts can have profound implications for cardiac function under conditions of increased workload, and therefore therapeutic approaches are warranted. Adropin is a liver- and brain-secreted peptide hormone shown to regulate fuel metabolism in the heart. Adropin levels are significantly reduced in obese and diabetic human subjects, and this decrease is linked to increased adiposity, insulin resistance, and impaired glucose tolerance. Acute Adropin treatment restores glucose oxidation activity in the hearts of prediabetic obese mice. However, the effects of chronic Adropin exposure in the heart remains unknown. A preclinical model of DCM was used to investigate the effects of long-term Adropin treatment on cardiac structural and metabolic remodeling. Mice were treated with Adropin daily for four weeks, and subjected to echocardiography, glucose tolerance tests, histopathology, RNA-sequencing, and metabolomics. Long-term Adropin treatment restores normal cardiac structure and metabolic function in the diabetic heart, by inhibiting flux of non-oxidative glycolytic intermediates into the hexosamine biosynthetic pathway. These findings highlight the therapeutic use of Adropin signaling to attenuate cardiac remodeling in DCM.

Treatment for HFpEF has centered around the management of comorbidities (e.g. obesity, hypertension), with current guidelines recommending the use of glycemia-reducing therapeutics that improve cardiovascular outcomes (e.g. SGLT2 inhibitors). A recent STEP-HFpEF trial of obesity-related HFpEF demonstrates that therapy-driven reductions in body weight are also a key tool in disease regression. These developments show that new approaches targeting metabolic changes in HFpEF offer substantial therapeutic benefits. Acute treatment with recombinant Adropin, a liver-derived peptide hormone, was previously demonstrated to restore cardiac glucose oxidation in high fat diet-fed mice. In this study, the therapeutic potential of long-term Adropin treatment was investigated in a preclinical model of HFpEF.

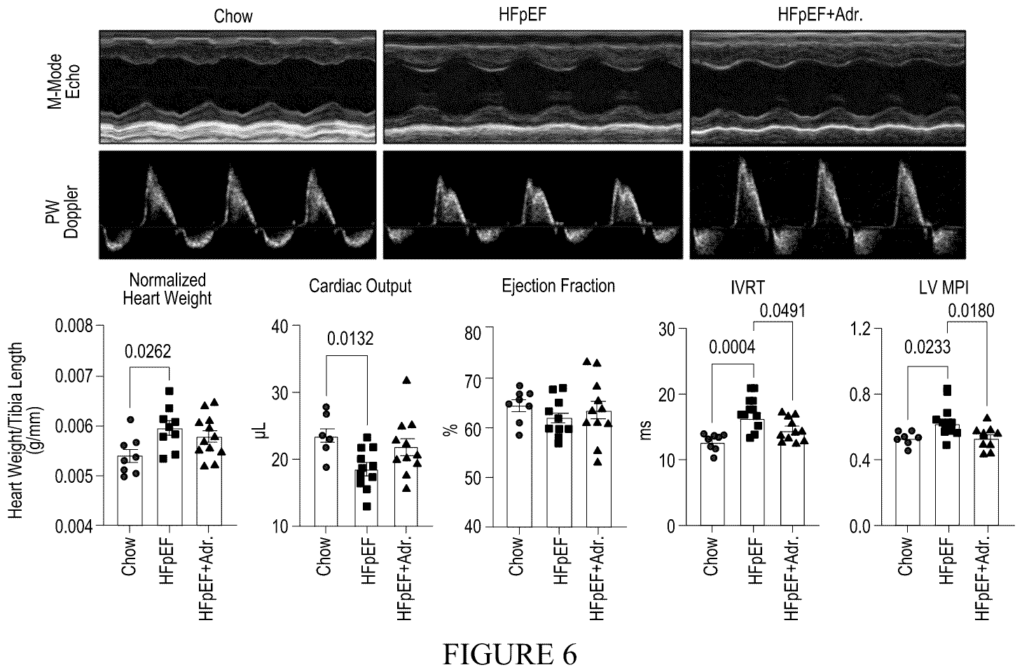

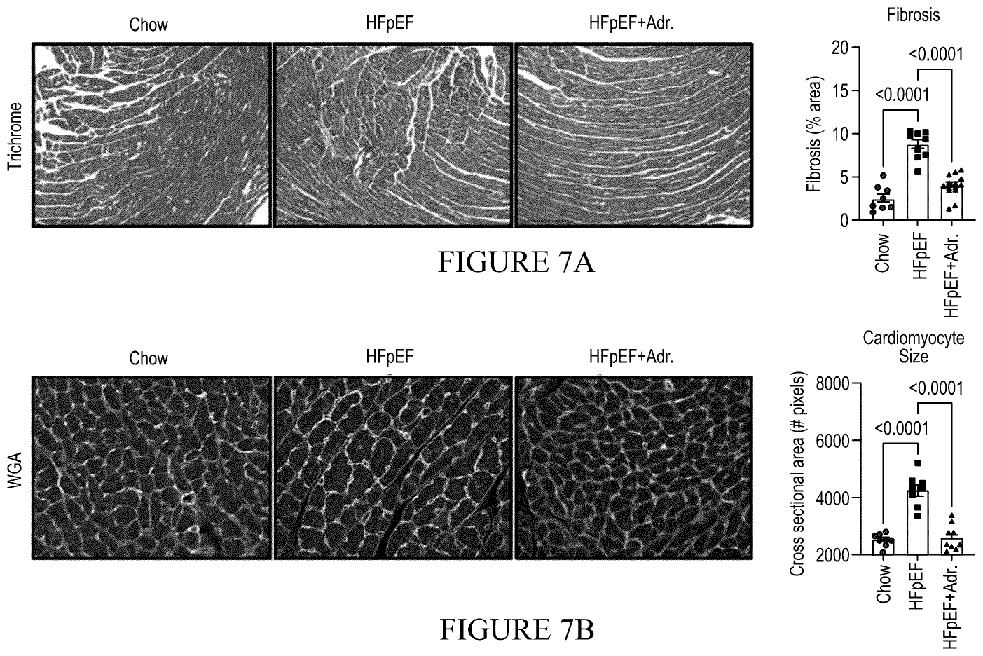

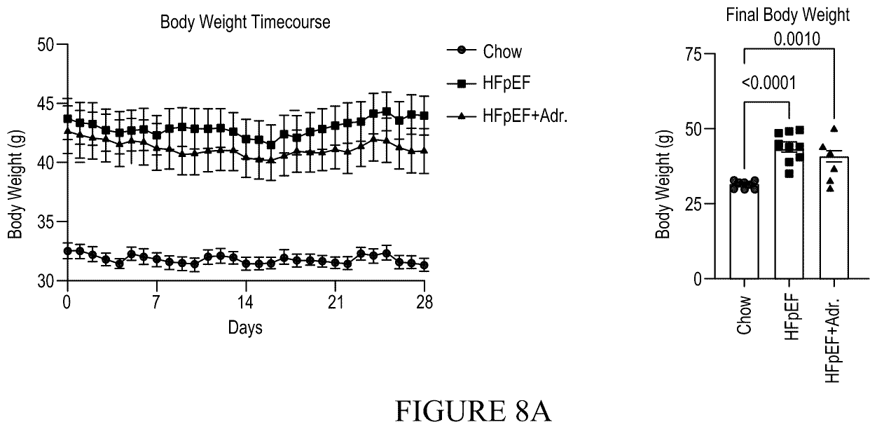

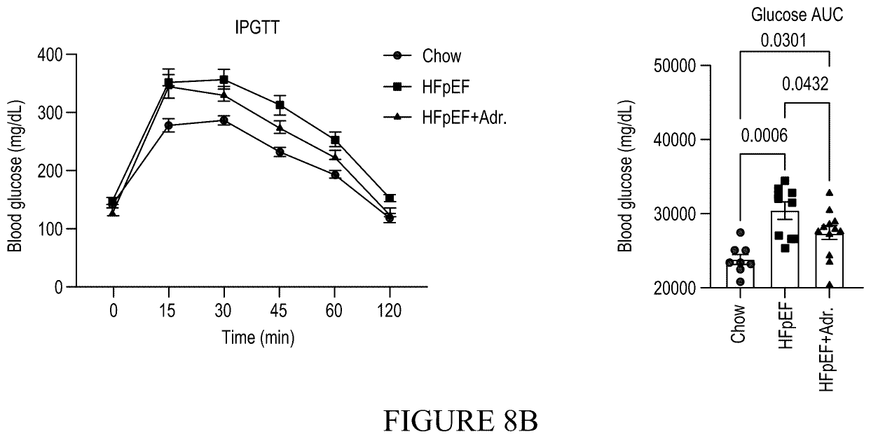

Twelve week-old C57BL/6J wild-type male mice were exposed to normal chow, or a combination of 60% high fat diet plus N^{ω}-nitro-L-arginine methyl ester (L-NAME: 0.5 g/L in drinking water) to induce HFpEF. After 8 weeks of diet, mice were randomly assigned to a control HFpEF group, or to a group receiving daily intraperitoneal injections of recombinant Adropin (450 nmol/kg) for the remaining weeks of the study. All animal experiments were approved by the University of Pittsburgh Institutional Animal Care and Use Committee. Echocardiographic evaluation revealed that HFpEF mice developed cardiac dysfunction, as evidenced by increased heart mass, decreased cardiac output, increased isovolumetric relaxation time (IVRT), and increased left ventricular myocardial performance index (LV MPI) (FIG. 6). These changes occurred in the absence of systolic dysfunction (FIG. 6). Each marker of cardiac dysfunction was absent in HFpEF mice treated with Adropin (FIG. 6). Histological analysis revealed that Adropin treatment prevented the development of fibrosis and cardiomyocyte hypertrophy in mice exposed to a HFpEF diet (FIGS. 7A and 7B). The improvements in cardiac structure and function observed with Adropin treatment were not associated with a significant decrease in absolute body weight (FIG. 8A), as observed in the STEP-HFpEF trial with semaglutide. Instead, Adropin treatment significantly restored whole-body glucose tolerance (FIG. 8B), showing that improvements in cardiac outcomes are related to metabolism.

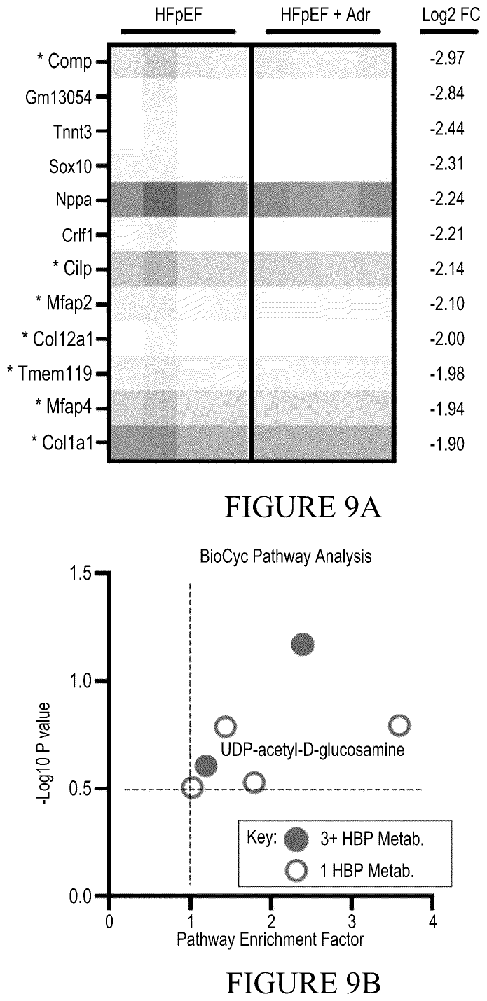

To understand Adropin-mediated changes in cardiac metabolism, transcriptomic and metabolomic analyses of hearts from control HFpEF and Adropin treated HFpEF mice were performed. Bulk RNA-seq showed no widespread changes in metabolic genes between HFpEF and Adropin groups; significant changes were restricted to decreased expression of extracellular matrix remodeling/fibrosis (e.g. Comp, Col12a1) and natriuretic peptide (e.g. Nppa) genes in Adropin-treated mice (FIG. 9A). Untargeted BioCyc pathway analysis revealed that metabolites related to UDP-N-acetyl-D-glucosamine (UDP-GlcNAc) biosynthesis represented the major metabolomic differences between the two groups (FIG. 9B), implicating modulation of the hexosamine biosynthesis pathway (HBP) as the underlying mechanism of Adropin activity in HFpEF. The HBP is a nutrient sensing signaling pathway that coordinates flux through major energy metabolism pathways, integrating glucose, amino acid, fatty acid, and nucleotide metabolism to generate UDP-GlcNAc, the donor substrate for protein O-GlcNAcylation. While studies have implicated aberrant regulation of HBP and consequent maladaptive protein O-GlcNAcylation in diabetic heart disease, its role in HFpEF has not previously been determined.

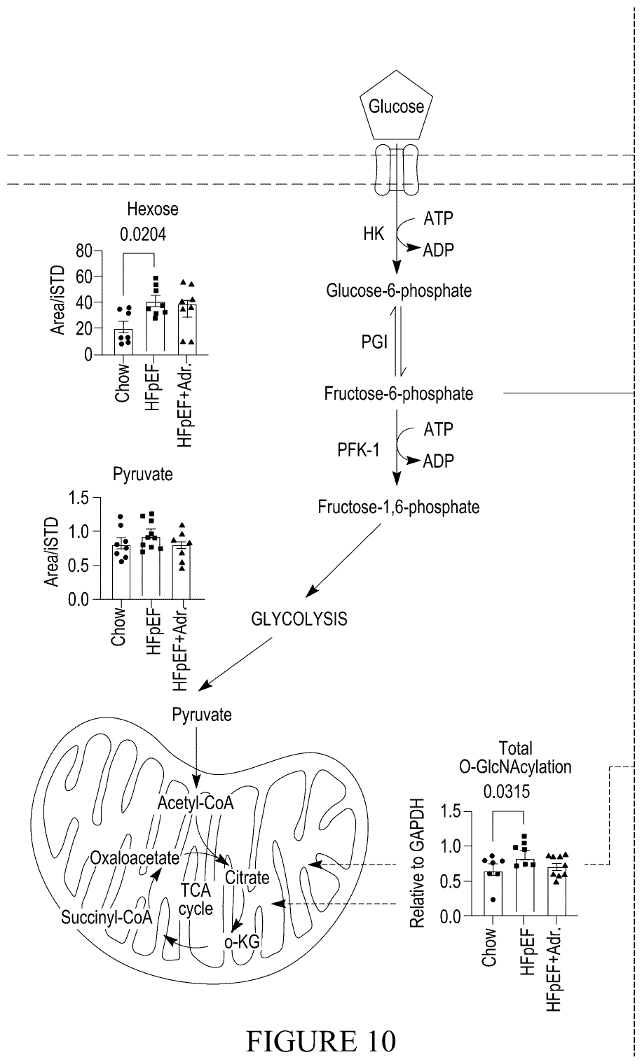

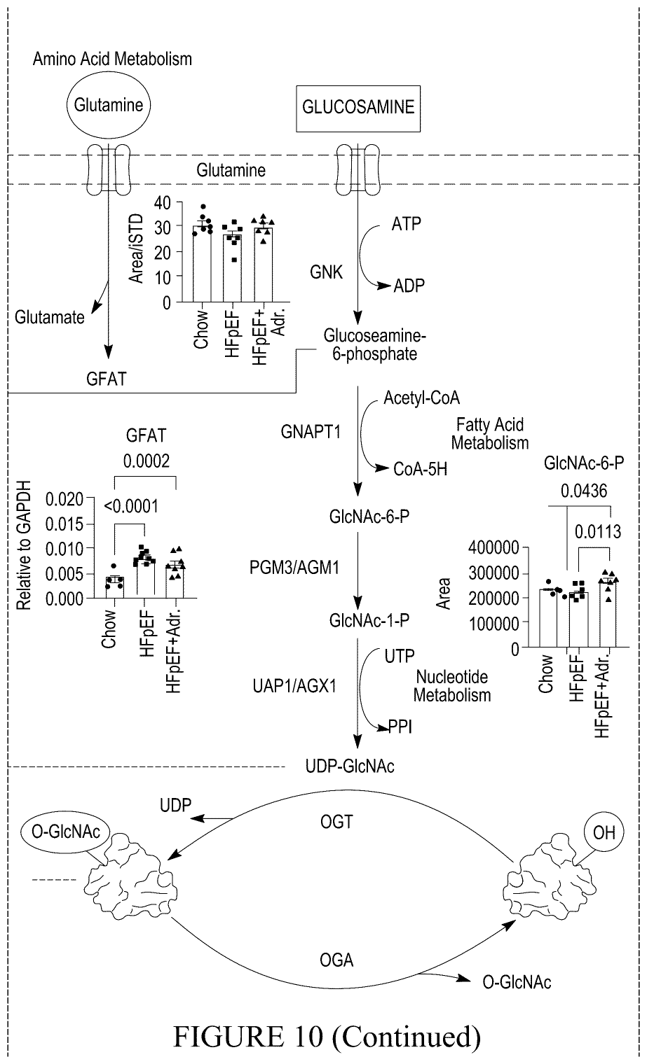

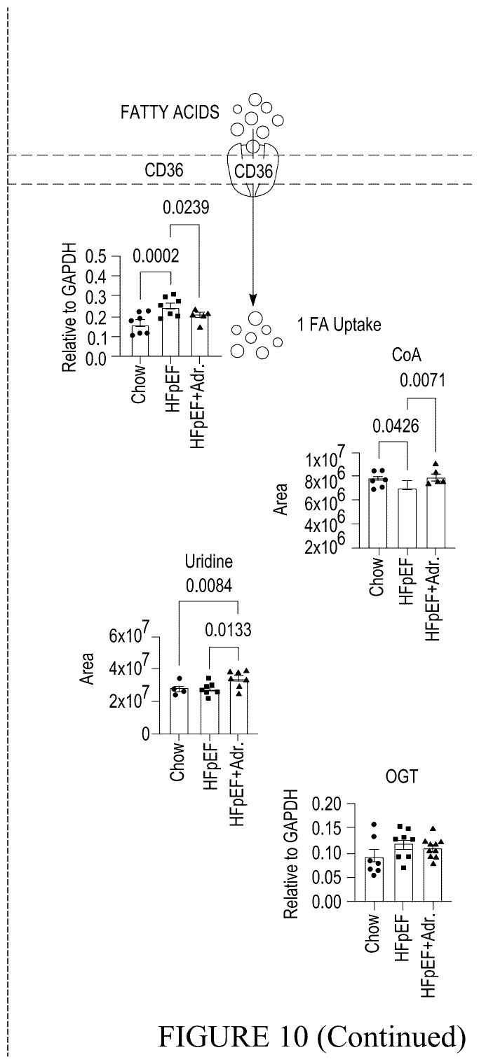

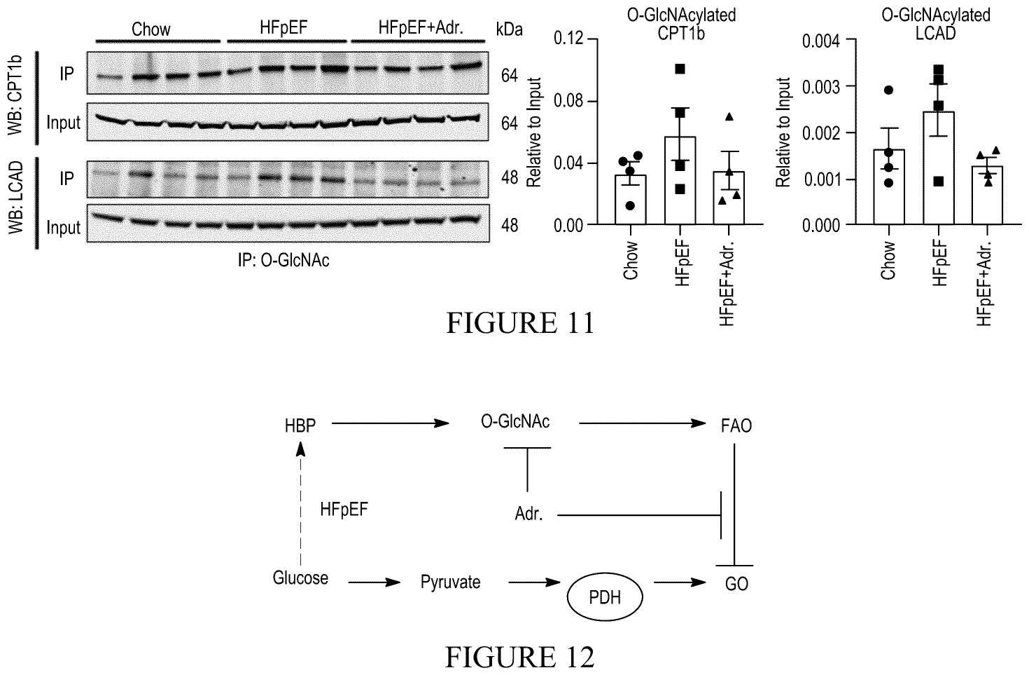

Metabolomic and biochemical analyses show that HBP activity was significantly increased in HFpEF mice, as shown by increased expression of the rate-limiting HBP enzyme GFAT, and a significant increase in total cardiac protein O-GlcNAcylation (FIG. 10). Additionally, there was a significant increase in the plasma fatty acid transporter CD36 in HFpEF mice, which exacerbates HBP activity via the provision of fatty acid oxidation substrates. While Adropin treatment did not reduce GFAT expression, it restored CD36 abundance and total cardiac protein O-GlcNAcylation to levels observed in chow mice (FIG. 10). Importantly, these data show that Adropin also inhibited cardiac protein 0-GlcNAcylation by decreasing substrate entry from several metabolic pathways that contributes to the HBP. Adropin treatment led to significant increase in the abundance of metabolites or by-products used in the generation of UDP-GlcNAc (such as GlcNAc-6-P, uridine, and coenzyme A), showing a decrease in their use for UDP-GlcNAc biosynthesis (FIG. 10). Combined with a trend towards decreased cardiac hexose and pyruvate levels, these data show that Adropin treatment reduces metabolite entry into the HBP, thereby restoring the use of glycolysis intermediates for oxidative energy metabolism. Finally, co-immunoprecipitation experiments were performed to identify specific protein 0-GlcNAcylation targets, which found that the mitochondrial fatty acid import protein CPT1b, and the fatty acid oxidation enzyme LCAD, were both O-GlcNAcylated in HFpEF mice (FIG. 11). In both cases, treatment with Adropin restored O-GlcNAcylation levels to those observed in chow mice (FIG. 11).

In summary, in a preclinical model of HFpEF, glycolytic intermediates are shunted into the HBP, preventing their use in oxidative energy metabolism and driving excessive cardiac protein O-GlcNAcylation (FIG. 12). Treatment with Adropin reverses this metabolic remodeling and restores cardiac function. These findings highlight Adropin as a target for therapeutic development in the treatment of HFpEF.

### Example 4: The Hepatokine Adropin Protects Against Heart Failure with Preserved Ejection Fraction

Cardio-hepatic interactions play a crucial role in the pathophysiology of metabolic diseases, where perturbations in metabolic homeostasis significantly contribute to disease progression. The interplay between the heart and liver is mediated by metabolic signaling pathways, with deleterious changes leading to insulin resistance, inflammation, and oxidative stress. Therapeutic interventions targeting metabolic derangements in the cardio-hepatic axis hold promise in mitigating inter-organ dysfunction. In this study, a preclinical mouse model that recapitulates cardiometabolic heart failure with preserved ejection fraction (HFpEF) was used to elucidate the molecular mechanisms driving cardio-hepatic dysfunction. Whether long-term treatment with a recombinant hepatokine peptide hormone, adropin, could improve cardio-hepatic dysfunction was tested. Markers of HFpEF severity significantly correlate with hepatic injury characteristic of metabolic dysfunction-associated fatty liver disease/non-alcoholic fatty liver disease (MAFLD/NAFLD) progression. Metabolic dysregulation due to impaired fatty acid metabolism was identified as a major mechanism linking cardio-hepatic dysfunction in HFpEF. Long-term treatment with adropin reversed multiple markers of HFpEF-related cardiac dysfunction (including fibrosis, diastolic dysfunction, and cardiomyocyte hypertrophy), without improving hepatic outcomes. Using untargeted metabolomics we found that adropin treatment restricted metabolite entry the hexosamine biosynthesis pathway (HBP), leading to a reduction in the inhibitory O-GlcNAcylation of cardiac fatty acid oxidation enzymes. The progression of cardiac and hepatic disease in a preclinical mouse model of HFpEF results from dysfunction in shared metabolic pathways. Thus, targeting adropin signaling pathways is a novel therapeutic avenue for HFpEF treatment.

Heart failure (HF) is a complex clinical syndrome that results from any structural or functional impairments of ventricular filling or ejection of blood and remains the leading cause of morbidity and mortality worldwide [1]. Heart failure with preserved ejection fraction (HFpEF), a subtype of heart failure, is primarily characterized by diastolic dysfunction. This presentation of the disease currently accounts for >50% of all heart failure cases, with a global prevalence of approximately 32 million that continues to rise at a rate of 1% per year [3, 4]. HFpEF is recognized as a heterogenous syndrome whose underlying pathophysiological mechanisms include several extracardiac abnormalities, such as metabolic derangements, arterial hypertension, microvascular endothelial dysfunction, inflammation, and renal insufficiency [3, 4]. The phenotypic diversity presented in HFpEF involves functional and structural changes in several organs including the kidneys, brain, liver, intestines, lungs, and adipose tissue [2], suggesting that inter-organ crosstalk during heart failure can represent a promising opportunity to create new treatment paradigms. Recent position papers from both the National Heart, Lung, and Blood Institute (NHLBI) and European Society of Cardiology (ESC) Working Group on Myocardial Function reinforced the urgency of investigating fundamental mechanisms underlying inter-organ interactions in heart failure, to facilitate the development of therapeutic strategies whose clinical applications restore systemic homeostasis in patients [2, 4].

Metabolic dysfunction-associated fatty liver disease (MAFLD), formerly known as non-alcoholic fatty liver disease (NAFLD), is considered the hepatic manifestation of the metabolic syndrome. The newly proposed criteria for a diagnosis of MAFLD includes hepatic steatosis, in addition to the presence of obesity, type 2 diabetes mellitus (T2DM), or evidence of metabolic dysregulation defined by at least two metabolic risk abnormalities [14]. Phenomapping analysis identified a metabolic phenotype of HFpEF characterized in obese, diabetic patients with obstructive sleep apnea and extreme delay in LV relaxation [15], which was later proposed as a distinct cardiac manifestation of NAFLD [16]. Numerous cross-sectional studies and meta-analyses revealed a significant association between left ventricular (LV) diastolic dysfunction and NAFLD in T2DM populations using echocardiography parameters [17-19], coupled with impaired myocardial metabolism [20-22], and adverse structural remodeling [23, 24]. This association strengthened with the progression of NAFLD to non-alcoholic steatohepatitis (NASH), correlating to worsened diastolic dysfunction [25, 26]. Surprisingly, despite the clear clinical links between HFpEF and NAFLD/MAFLD, there are almost no preclinical animal studies that explore this relationship or attempt to elucidate the potential mechanisms underlying inter-organ crosstalk.

Treatment for HFpEF currently focuses on the management of comorbidities (e.g. obesity, type 2 diabetes, hypertension), with new guidelines recommending the use of glycemia-reducing therapeutics (e.g. SGLT2 inhibitors) that improve cardiovascular outcomes in heart failure [73]. The recent STEP-HFpEF trial of obesity-related HFpEF demonstrated that therapy-mediated reductions in body weight can also be a key tool in disease regression [74]. These developments suggest that approaches targeting metabolic changes can offer substantial treatment benefits in HFpEF, leading to the search for new therapeutic targets. Previous studies have shown that adropin, a brain- and liver-derived peptide hormone, improved physical activity [6-8], attenuated hepatic steatosis and injury [7, 34], and reduced vascular stiffness in diet-induced obesity [9]. Furthermore, short-term adropin treatment can improve cardiac fuel substrate flexibility by restoring glucose oxidation in the diabetic heart [30]. Therefore, this peptide was investigated to determine whether it can be used as a therapeutic intervention for multi-organ dysfunction in HFpEF.

### Methods

Animal Care and Use. Male C57BL/6J 8-week-old mice were obtained from the Jackson Laboratory and maintained on chow for 4 weeks to acclimate to their new environment. Animals were housed in the University of Pittsburgh animal facility under standard conditions with ad libitum access to water and food and maintained on a constant 12-hour light/dark cycle. At 12 weeks of age, the mice were exposed to normal chow (Chow; 60% carbohydrate, 26% protein, 14% fat), or a combination of high-fat diet (HFD; 20% carbohydrate, 20% protein, 60% fat) plus No-nitro-L-arginine methyl ester (L-NAME: 0.5 g/L) supplemented in their drinking water after adjusting the pH to 7.4 with 1N NaOH to induce HFpEF [10]. For cohort 1, the mice were subject to the HFpEF diet for 15 weeks. For cohort 2, after 8 weeks of diet, mice were randomly assigned to a control or treatment group receiving daily intraperitoneal injections of either vehicle or recombinant adropin at 450 nmol/kg animal weight prepared in PBS with 0.1% BSA for the remaining 5 weeks of the study. L-NAME supplemented drinking water was changed weekly and adropin was made fresh and sterile filtered before each use.

Animals were euthanized by isofluorane anesthesia, followed by cervical dislocation and cardiac tissue was excised after drawing blood from the right ventricle (RV) that was spun at 10,000 rpm for 1 minute to obtain plasma for analysis. Experiments were conducted in compliance with National Institutes of Health guidelines and followed procedures approved by the University of Pittsburgh Institutional Animal Care and Use Committee.

Echocardiography. Mice were anesthetized using isofluorane (1.5%-2.0% v/v by inhalation) and monitored for cardiac functional parameters in the supine position using a Visual Sonics Vevo 3100. Core temperature was maintained at 37° C. by imaging mice while on a heating pad, and heart rates were kept consistent between experimental groups (˜400-500 beats per min). Short axis M-mode was used to assess LV dimensions and motion patterns, pulsed-wave (PW) doppler mode to evaluate blood flow velocities across the mitral valve, and tissue doppler mode to measure mitral annular plane velocity. Doppler profiles were acquired in the parasternal long axis (PLAX) apical 4 chamber view. The left atrial area was quantified in the apical 4-chamber view by tracing the border of the left atrium. Markers of systolic and diastolic function were calculated using standard echocardiography equations. At the end of the procedure, all mice recovered from anesthesia without difficulties. Image analysis was performed independently by a blinded sonographer and all parameters were measured at least 3 times, and averages are presented.

Intraperitoneal Glucose Tolerance Test (IPGTT). Glucose tolerance tests were performed as previously described with minor modifications [11]. After an overnight fast (˜16 h), mice were set-up in restrainers with tails snipped <5 mm to prime for blood collection and left to acclimate under a heated bulb for 2 h prior to GTT start time at 9 a.m. Basal plasma samples (t=0) were collected, and D-glucose was diluted in water (20% final concentration) and sterile filtered for intraperitoneal administration at 1.5 g/kg body weight. Blood glucose was measured by tail bleed at set time points using a Bayer Contour Next EZ handheld glucometer (t=15, 30, 45, 60, and 120 min).

Histology. Preparation and staining of all histological samples were conducted by the Pitt Biospecimen Core at the University of Pittsburgh. All mice were sacrificed, and tissue harvested for analysis within one hour of completion of the IPGTT study. The apex of the heart and right (cohort 1) or left (cohort 2) lobe of the liver was excised from each mouse following euthanasia, fixed in 10% buffered formalin phosphate overnight at shaking incubation, washed 3 times for 5 min with 1× phosphate-buffered saline (PBS), and then transferred to 70% ethanol. Samples were then embedded in paraffin, sectioned into 4 μm slides and stained with hematoxylin and eosin (H&E), wheat germ agglutinin (WGA) or Masson's Trichrome for analysis. The NAFLD/MAFLD activity score (NAS) was obtained based on methods established by the Pathology Committee of the Non-alcoholic Steatohepatitis (NASH) Clinical Research Network [12]. Liver pathology scoring was performed in a blinded independent fashion. NAS consisted of separate category scores which included steatosis (0-3), where 0=0-5%, 1=6-33%, 2=34-66%, and 3=67-100% of hepatocytes positive for steatosis; lobular inflammation (0-3), where 0=no foci, 1=more than 2 foci, 2=2-4 foci and 3=greater than 4 foci all per 200× field; and hepatocellular ballooning (0-2), where 0=none present, 1=few and 2=many/prominent. The final NAS represents a sum of the three category scores. Histology sections were visualized with the Evos FL Auto 2 Microscope, observed changes in structure were quantified using ImageJ software and representative images are shown.

Transcriptomics. Total RNA was isolated from myocardial LV and hepatic tissue using the RNeasy Plus Mini Kit. Approximately 2 μg RNA was used for bulk RNA sequencing performed by Azenta/GENEWIZ based on company recommendations to identify differential gene expression patterns between the experimental groups.

Quantitative Metabolomics. Metabolic quenching and polar metabolite pool extraction was performed by adding ice cold 80% methanol (aqueous) at a ratio of 1:15 wt input tissue:vol. (13 C1)-creatinine, (D3)-taurine, (D3)-lactate and (D3)-alanine were added to the sample lysates as an internal standard for a final concentration of 10 pM. Samples are homogenized using an MP Bio FastPrep system using Matrix D (ceramic sphere) for 60 seconds at 60 hz. The supernatant was then cleared of protein by centrifugation at 16,000 g. 2 μL of cleared supernatant was subjected to online LC-MS analysis. Analyses were performed by untargeted liquid chromatography-high-resolution mass spectrometry (LC-HRMS). Briefly, samples were injected via a Thermo Vanquish UHPLC and separated over a reversed phase. Thermo HyperCarb porous graphite column (2.1×100 mm, 3 m particle size) maintained at 55° C. For the 20 min LC gradient, the mobile phase consisted of the following: solvent A (water/0.1% FA) and solvent B (ACN/0.1% FA). The gradient was the following: 0-1 min 1% B, increase to 15% B over 5 min, continue increasing to 98% B over 5 min, hold at 98% B for five min, re-equillibrate at 1% B for five min. The Thermo IDX tribrid mass spectrometer was operated in both positive and negative ion mode, scanning in ddMS2 mode (2 scans) from 70 to 800 m/z at 120,000 resolution with an AGC target of 2e5 for full scan, 2e4 for ms2 scans using HCD fragmentation at stepped 15, 35, 50 collision energies. Source ionization setting was 3.0 and 2.4 kV spray voltage, respectively, for positive and negative mode. Source gas parameters were 35 sheath gas, 12 auxiliary gas at 320° C., and 8 sweep gas. Calibration was performed prior to analysis using the Pierce™ FlexMix Ion Calibration Solutions. Integrated peak areas were then extracted manually using Quan Browser (Thermo Fisher Xcalibur ver. 2.7). Untargeted differential comparisons were performed using Compound Discoverer 3.0 to generate a ranked list of significant compounds with tentative identifications from BioCyc, KEGG, and internal compound databases. Purified standards were then purchased and compared in retention time, m/z, along with ms2 fragmentation patterns to validate the identity of significant hits.

Protein Isolation and Immunoblotting. Heart tissues were rapidly harvested following euthanasia, weighed, RV discarded and LV flash-frozen in liquid nitrogen. For protein isolation, tissues were minced and lysed in CHAPS buffer (1% CHAPS, 150 mM NaCl, 10 mM HEPES, pH 7.4) using a VWR 4-Place Mini Bead Mill, then incubated on ice for ˜2.5 h. Homogenates were spun at 10,000 g at 4° C. for 10 min, and the supernatants collected for immunoblotting. For immunoblotting, protein lysates were quantitated using a BioDrop LITE Analyzer, prepared in LDS sample buffer, separated using Bolt SDS-PAGE 4-12% or 12% Bis-Tris Plus gels, and transferred to nitrocellulose membranes. Membranes were blocked using SuperBlock (PBS) Blocking Buffer and incubated overnight in the following primary antibodies: rabbit PDK4 (PA5-13776); mouse 0-GlcNAc (9875), rabbit GFAT (D12F4), rabbit OGT (D1D8Q), rabbit OGA (E9C5U), rabbit GAPDH (2118S); rabbit CD36 (18836-1-AP), rabbit CPT1b (22170-1-AP), rabbit LCAD (17526-1-AP), and GCN5L1 as previously reported (Scott et al., 2012). Protein loading was confirmed using GAPDH as a loading control. Fluorescent anti-goat or anti-rabbit secondary antibodies (red, 700 nm; green, 800 nm) were used to detect expression levels. Images were obtained using Licor Odyssey CLx System and protein densitometry was measured using the LiCor Image Studio Lite Ver. 5.2 Software.

Co-Immunoprecipitation. For co-immunoprecipitation experiments, protein lysates were harvested in CHAPS lysis buffer (1% CHAPS, 150 mM NaCl, 10 mM HEPES, pH 7.4), and 500 μg of protein were incubated overnight at 4° C. with 5 μL mouse O-GlcNAc antibody. Immunocaptured proteins were isolated using Protein-G and Protein-A agarose beads, washed 5 times with 500 μL 1% CHAPS buffer, and then eluted in LDS sample buffer at 95° C. Samples were separated on 4-12% Bis-Tris Bolt gels, transferred to nitrocellulose membranes, and probed with appropriate antibodies. Images were obtained using LiCor Odyssey CLx System and protein densitometry was measured using the LiCor Image Studio Lite Ver. 5.2 Software.

LCAD Activity Assay/Biochemical Assays/Enzyme activity assay. To assess the activity of long chain acyl-CoA dehydrogenase enzymes (LCAD), homogenized protein samples were incubated with palmitoyl-CoA as described previously [13]. Briefly, ˜10 g of protein was incubated with 0.1 M potassium phosphate, 50 pM 2,6-dichlorophenolindophenol, 2 mM phenazine ethosulfate, 0.2 mM N-ethylmaleimide, 0.4 mM potassium cyanide, and 0.1% Triton X-100 at 37° C. for 4 min. The reaction was initiated with 60 M acyl-CoA, and the rate of absorbance change was measured/observed at 600 nm over 45 min. Activities were converted to moles of substrate oxidized/min/mass of protein. In vitro LCAD activity was measured after incubating a reaction mixture of 4 ug of recombinant LCAD, 0.8 mM UDP-GlcNAc, 40 ug/mL of recombinant human O-GlcNAc Transferase (rhOGT) in assay buffer (25 mM Tris, 10 mM CaCl2), pH 7.5 and 10 mM MgCl2) at 37° C. for 30 min.

Statistics. Means±SEM were calculated for all data sets. Data were analyzed using either one-way ANOVA or two-way ANOVA with Tukey's post-hoc testing to determine differences between genotypes and feeding/treatment groups. Time course data was analyzed using two-way ANOVA with Sidak's post-hoc testing. P≤0.05 was considered statistically significant. Statistical analyses were performed using GraphPad Prism 9.5 Software.

### Results

Liver injury accompanies diastolic dysfunction in HFpEF. Metabolic dysfunction-associated fatty liver disease (MAFLD), formerly known as non-alcoholic fatty liver disease (NAFLD), is considered the hepatic manifestation of the metabolic syndrome. The newly proposed criteria for a diagnosis of MAFLD include hepatic steatosis in addition to the presence of obesity, type 2 diabetes mellitus (T2DM), or evidence of metabolic dysregulation defined by at least two metabolic risk abnormalities [14]. Phenomapping analysis identified a metabolic phenotype of HFpEF characterized in obese, diabetic patients with obstructive sleep apnea and extreme delay in LV relaxation [15], which was later proposed as a distinct cardiac manifestation of NAFLD [16]. Numerous cross-sectional studies and meta-analyses revealed a significant association between left ventricular (LV) diastolic dysfunction and NAFLD in T2DM populations using echocardiography parameters [17-19], coupled with impaired myocardial metabolism [20-22], and adverse structural remodeling [23, 24]. This association strengthened with the progression of NAFLD to non-alcoholic steatohepatitis (NASH), correlating to worsened diastolic dysfunction [25, 26]. Surprisingly, despite the clear clinical links between HFpEF and NAFLD/MAFLD, there are almost no preclinical animal studies that explore this relationship or attempt to elucidate the potential mechanisms underlying inter-organ crosstalk.

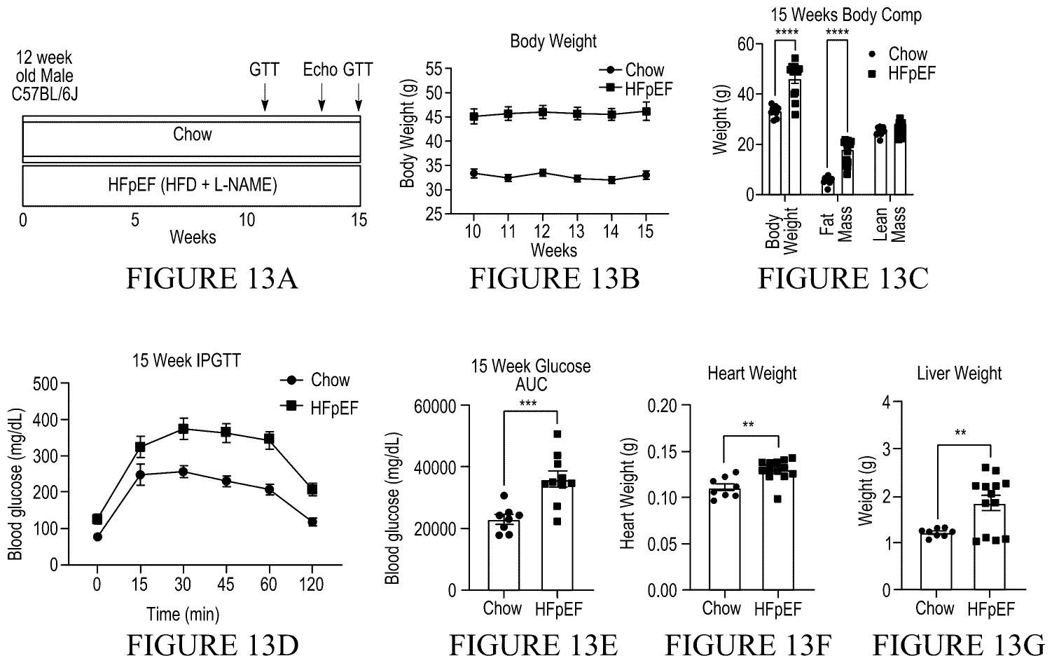

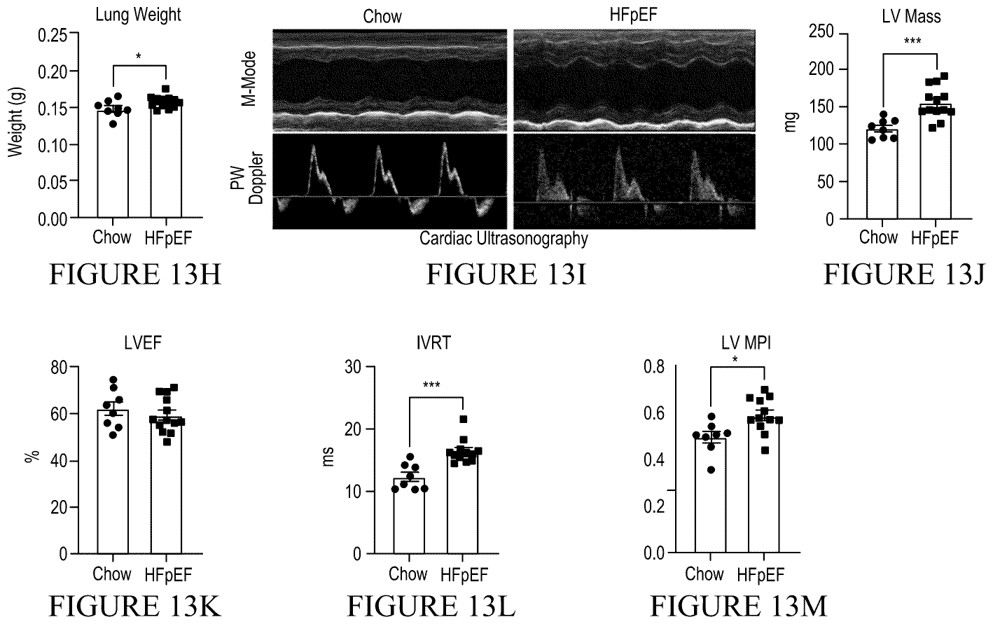

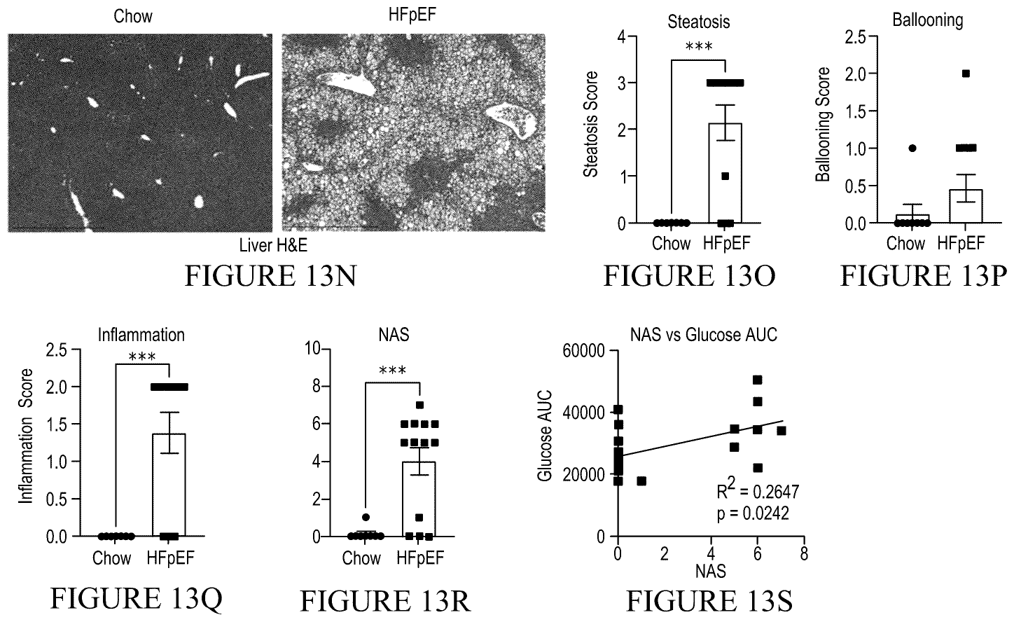

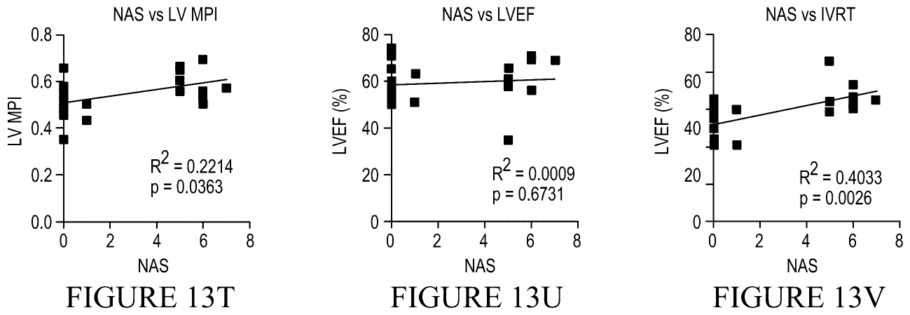

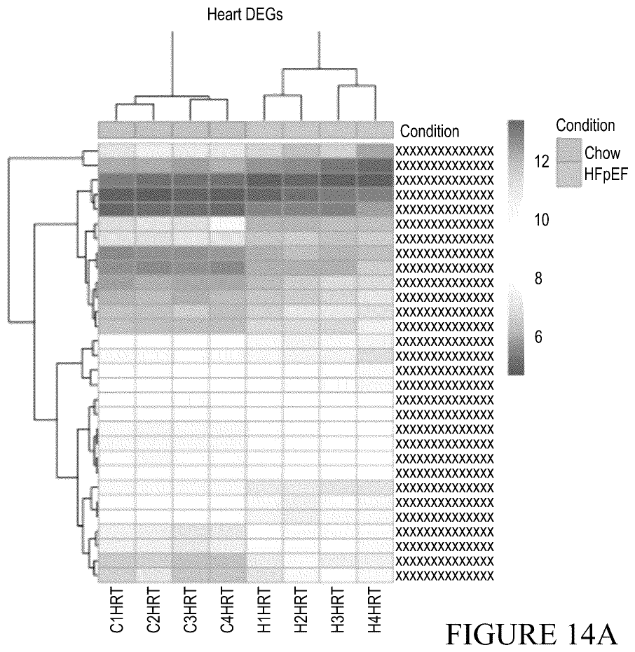

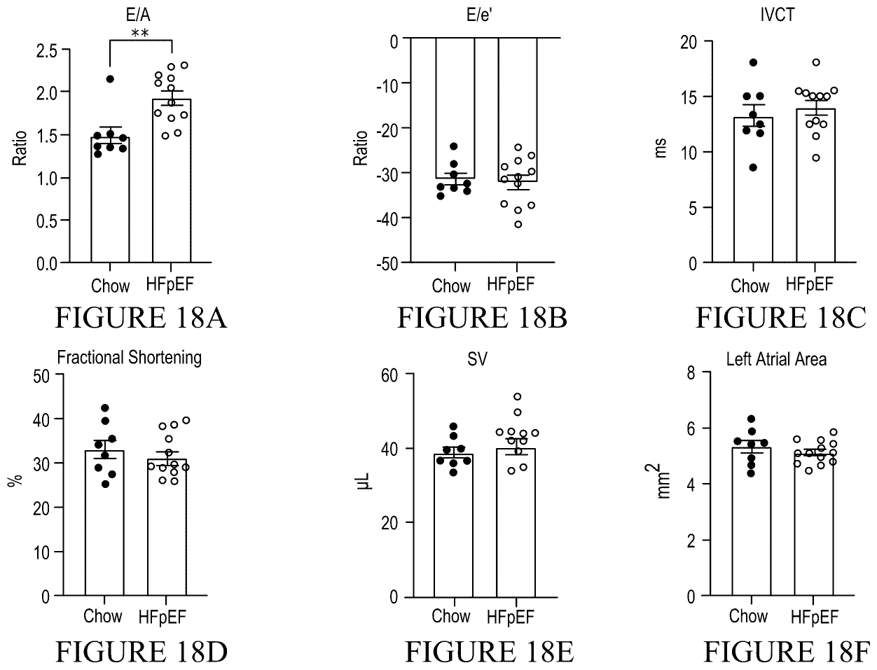

Utilizing a recently developed two-hit preclinical model of HFpEF [10], the presence of liver injury indicative of NAFLD/MAFLD development was assessed. Male C57BL/6J mice were subject to the HFpEF model (high fat diet+L-NAME) for 15 weeks (FIG. 13A), becoming obese due to a significant increase in fat mass (FIGS. 13B-13C). Intraperitoneal glucose tolerance tests (IPGTT) revealed a significant increase in circulating blood glucose levels compared to chow controls, indicating a T2DM-like state (FIGS. 13D-13E). HFpEF mice exhibited a significant increase in absolute heart weight (FIG. 13F), liver weight (FIG. 13G), and lung weight suggestive of pulmonary edema (FIG. 13H). Echocardiographic evaluation (FIG. 13I) revealed a significant increase in LV mass (FIG. 13J) coupled with preserved ejection fraction (FIG. 13K), increased isovolumetric relaxation time (IVRT) (FIG. 13L), and left ventricular myocardial performance index (LV MPI) (FIG. 13M), suggesting diastolic dysfunction in the absence of systolic dysfunction (FIGS. 18A-18F). In the liver, histological analyses by H&E staining (FIG. 13N) showed a significant increase in steatosis (FIG. 13O) and inflammation (FIG. 13Q) in the HFpEF mice compared to chow controls, suggesting the presence of NAFLD/MAFLD (FIG. 13R). Linear regression analysis revealed a significant positive correlation between whole-body circulating glucose levels and liver NALFD activity score (NAS) (FIG. 13S), confirming the association between insulin resistance and NAFLD/MAFLD. While there was no correlation with systolic function (FIG. 13T), there was significant positive correlation between LV diastolic dysfunction (IVRT, LV MPI) and liver NAS (FIGS. 13U-13V), suggesting that liver injury accompanies diastolic dysfunction in HFpEF. Together, these data are the first to report the coexistence of HFpEF and NAFLD/MAFLD in a preclinical mouse model, supporting previously published clinical data in HFpEF patients (Dong et al., 2020, Yong et al., 2022).

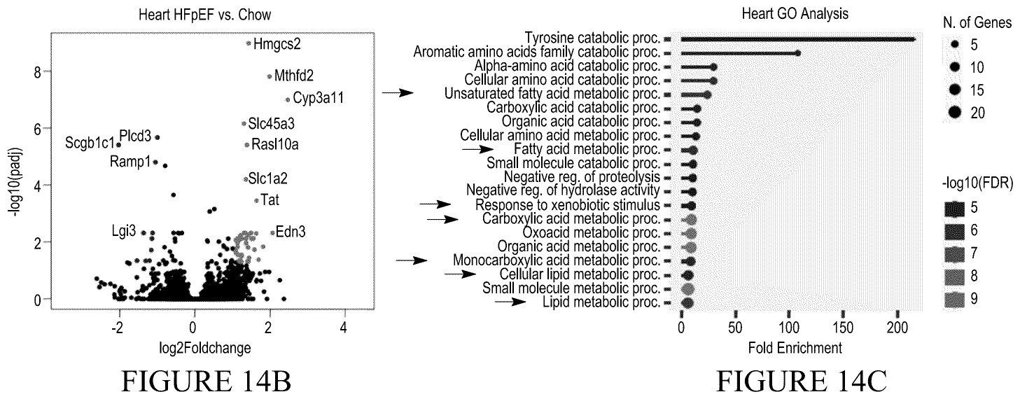

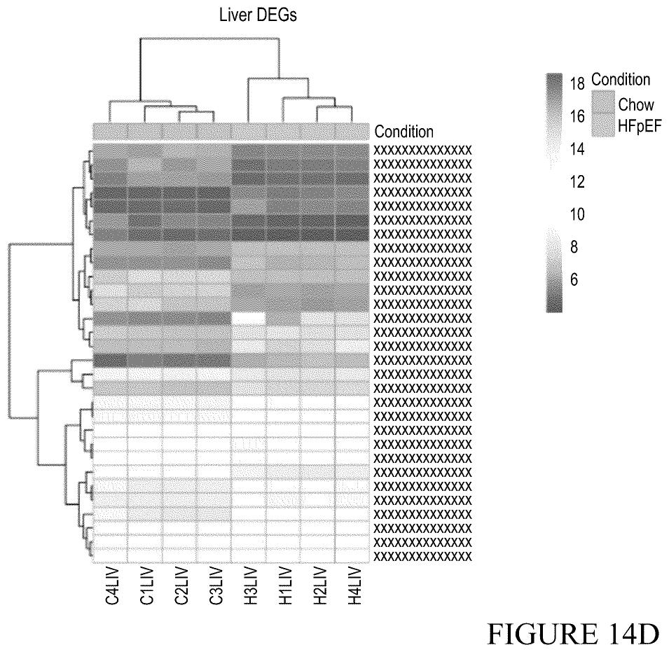

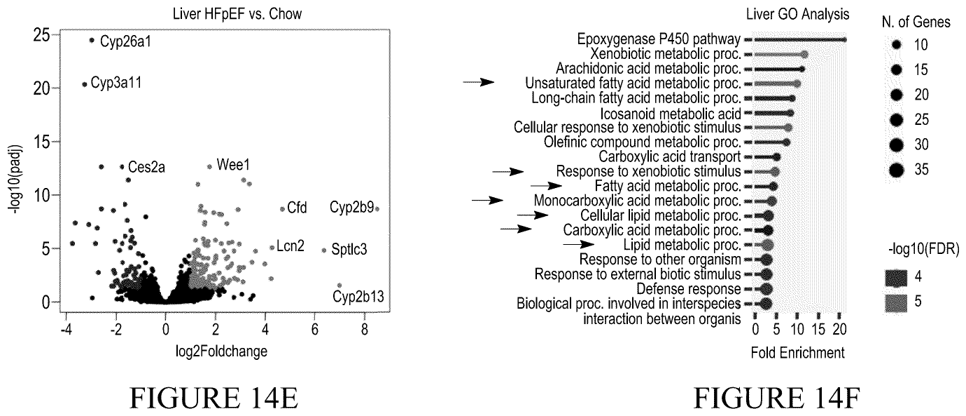

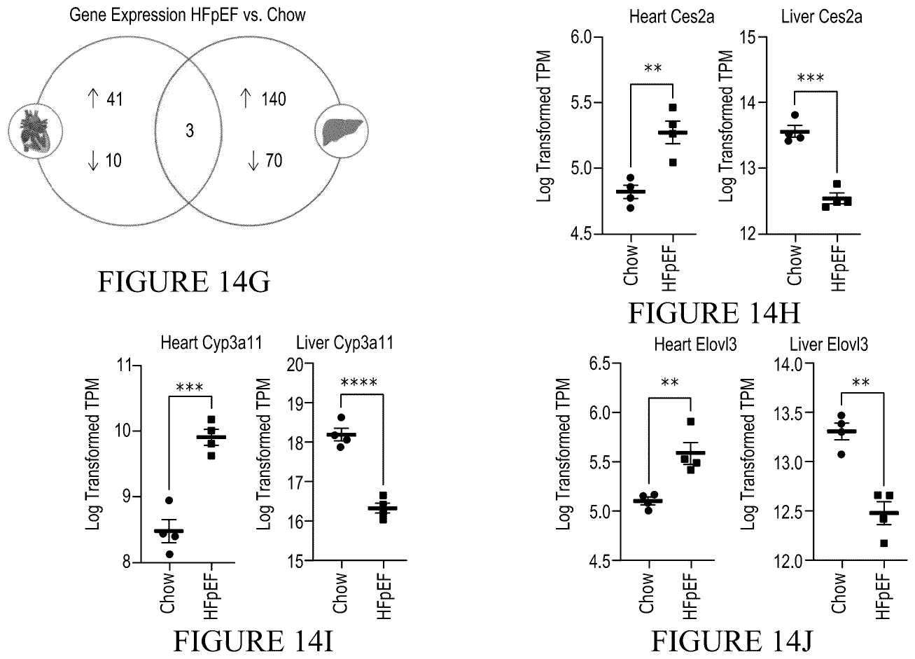

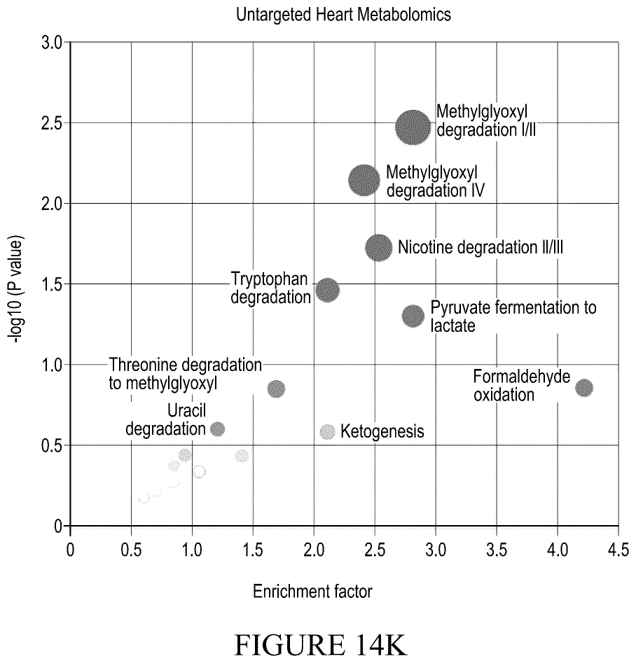

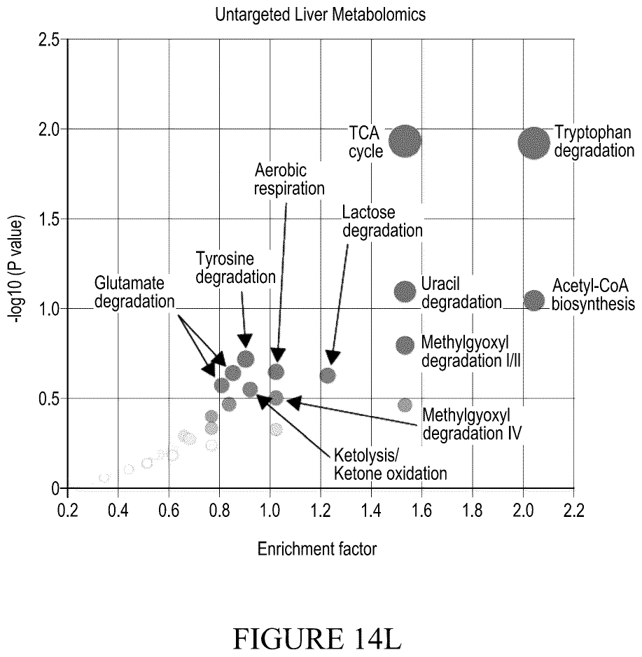

Impaired substrate utilization underlies cardio-hepatic metabolic dysregulation in HFpEF. Bulk-RNA sequencing revealed 51 differentially expressed genes (DEGs) in the HFpEF myocardium compared to the chow controls, with top hits including Hmgcs2, Mthfd2, Plcd3, Cenpf, and Gnb3 (FIGS. 14A-14C); all markers of cardiac metabolic dysfunction. In HFpEF hepatic tissue, 210 DEGs were identified, with significant hits including Cyp26a1, Cyp3a11, Cyp2b9, and Cyp2b13; all genes involved in the epoxygenase p450 pathway (FIGS. 14D-14F). GO analysis highlighted seven common biological processes between the HFpEF cardiac and hepatic tissue, including unsaturated fatty acid metabolic processes, fatty acid metabolic processes, and lipid metabolic processes (FIGS. 14C, 14F), highlighting metabolic dysregulation as a shared cardio-hepatic factor driving HFpEF development. Surprisingly, only three genes were differentially expressed in both the heart and liver tissues (FIG. 14G), all of which are associated with fatty acid metabolism. Specifically, Ces2a, Cyp3a11, and Elovl3 were significantly upregulated in the heart, but downregulated in the liver, indicating a divergent response to increased fat availability (FIGS. 14H-14J). Untargeted metabolomics revealed methylglyoxal degradation, pyruvate fermentation to lactate, and ketogenesis among the top enriched pathways in the heart (FIG. 14K), while the liver showed significant enrichment in TCA cycle, acetyl-CoA biosynthesis, and ketone oxidation pathways in HFpEF mice (FIG. 14L). Interestingly, the metabolomic signatures of heart and liver tissues in HFpEF mice shared three major pathways, including methylglyoxal degradation, tryptophan degradation, and uracil degradation (FIG. 14K-14L). Collectively, these data suggest that impaired energy substrate utilization is an underlying mechanism of metabolic dysregulation in the cardio-hepatic HFpEF axis.

Long-term adropin treatment restores cardiac function in HFpEF. Hepatokines play a crucial role in inter-organ interactions, mediating tissue-protective effects that counteract oxidative stress, inflammation, and mitochondrial dysfunction [27]. Adropin, a novel hepatokine, exhibits significantly reduced levels in obese and diabetic individuals [28]. This decrease has been linked to increased adiposity, impaired glucose homeostasis, hypertriglyceridemia, insulin resistance, and hepatosteatosis [7, 8]. Previous studies have demonstrated that adropin treatment modulates key intracellular signaling pathways to reduce hyperglycemia and restore cardiac glucose oxidation in diet-induced obese mice [29-31]. Acute adropin treatment has been shown to increase cardiac contractility in hearts isolated from chow-fed mice [32], however a recent study demonstrated that this effect is lost after exposure to a long-term HFD in ex vivo perfusion studies [33]. Thus, chronic adropin treatment was investigated to determine whether any protection is conferred from contractile dysfunction in vivo following HFpEF induction.

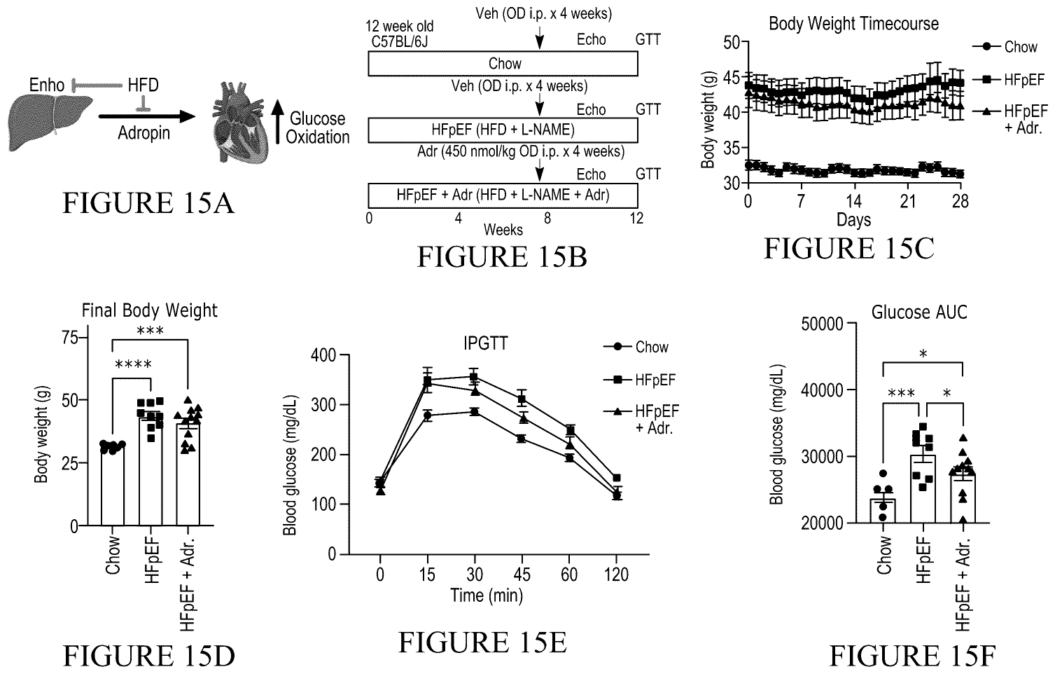

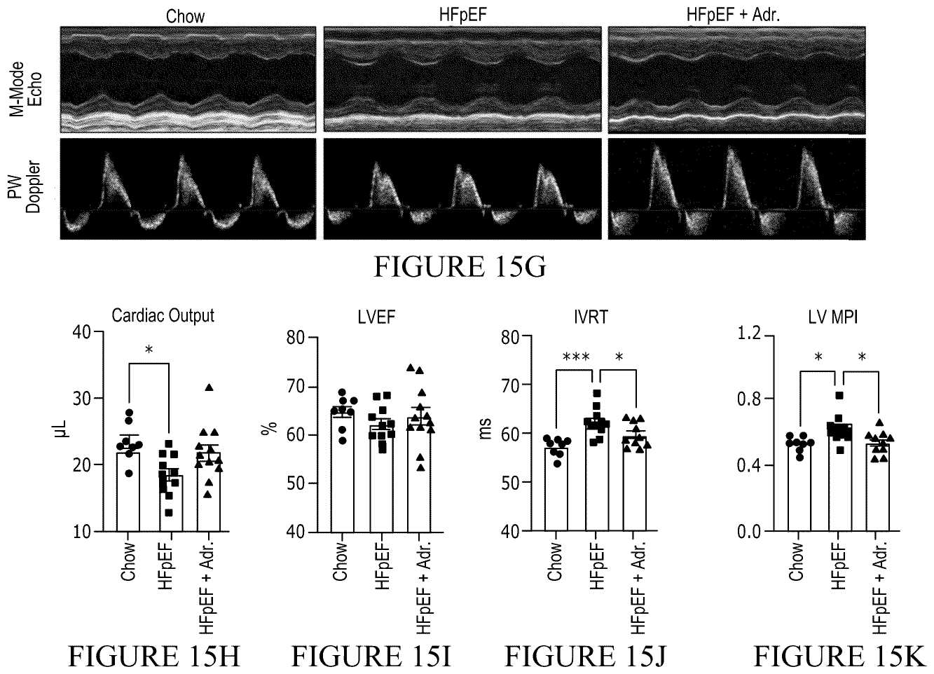

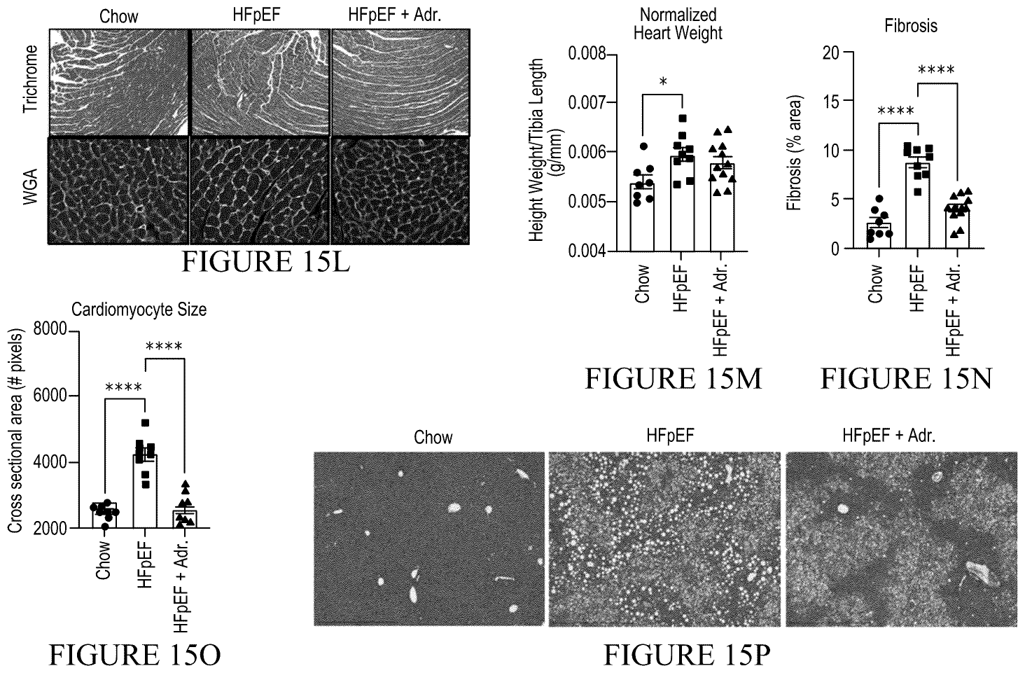

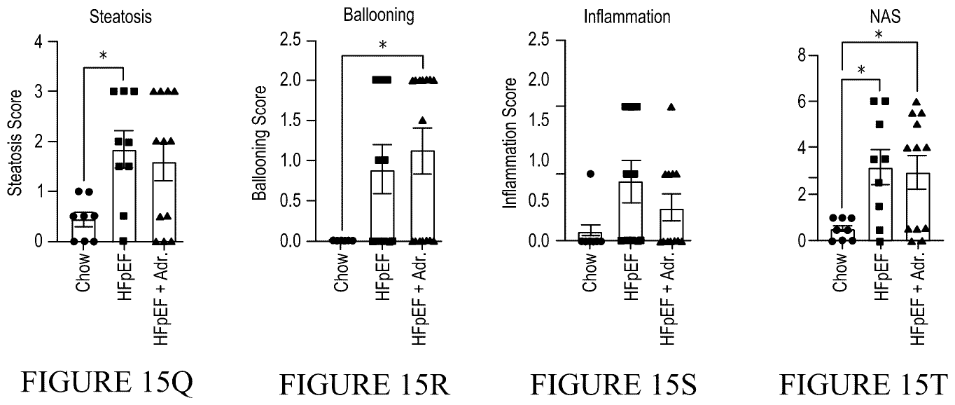

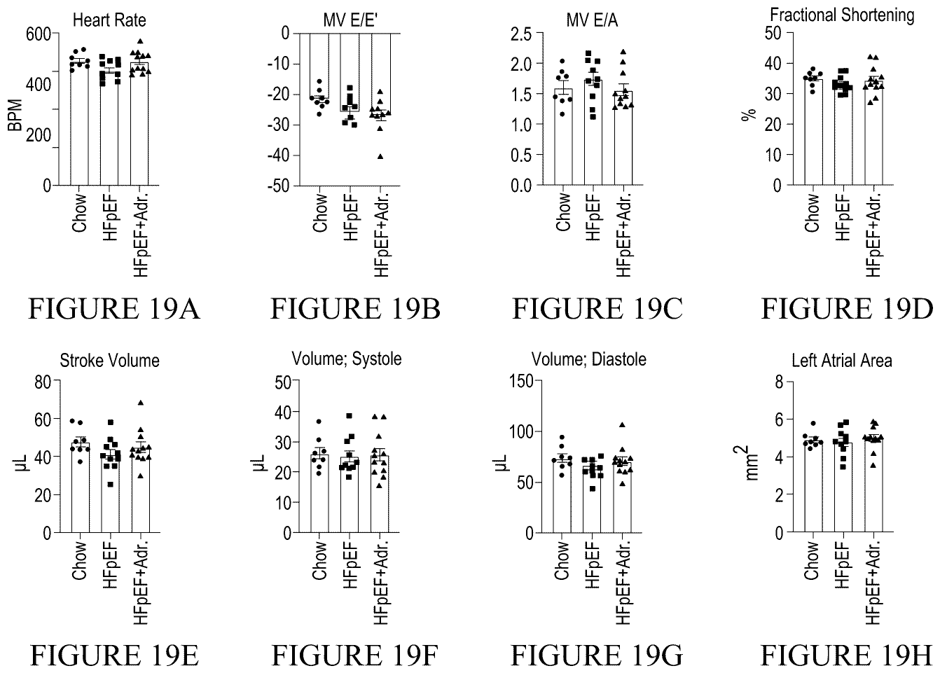

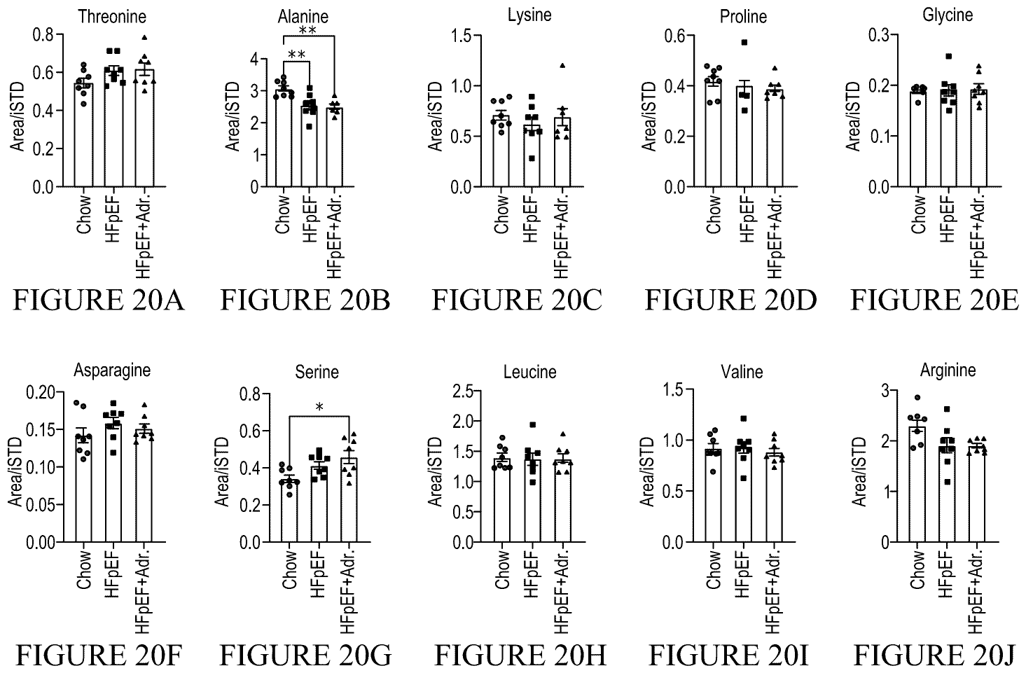

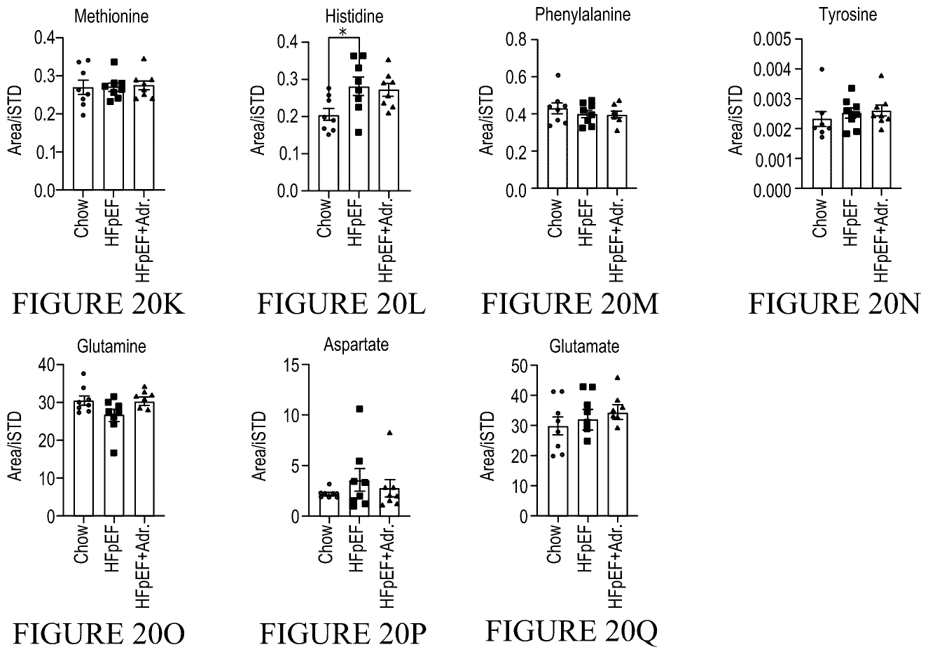

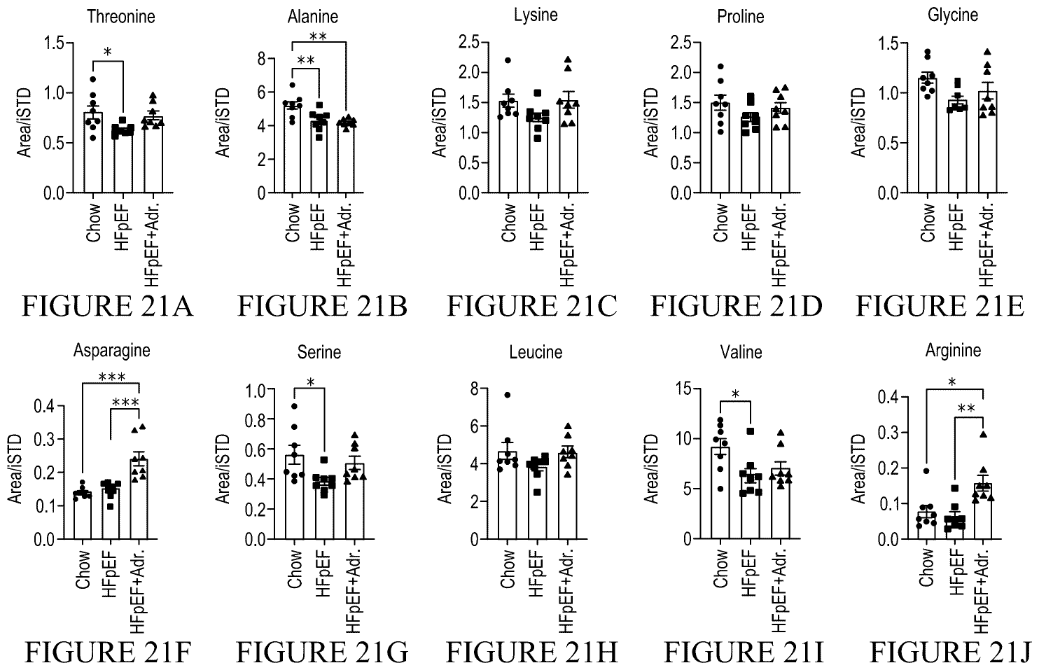

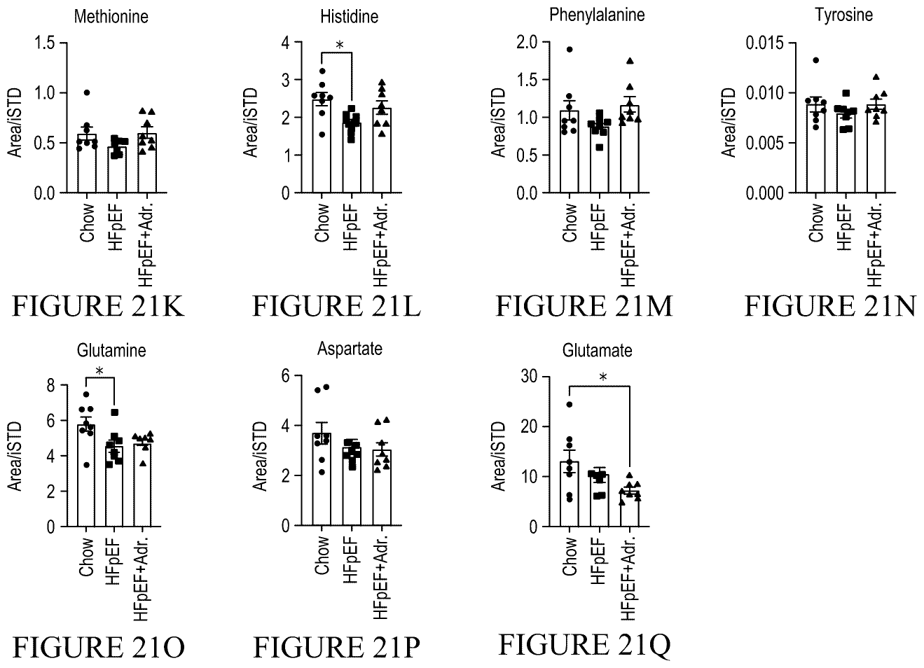

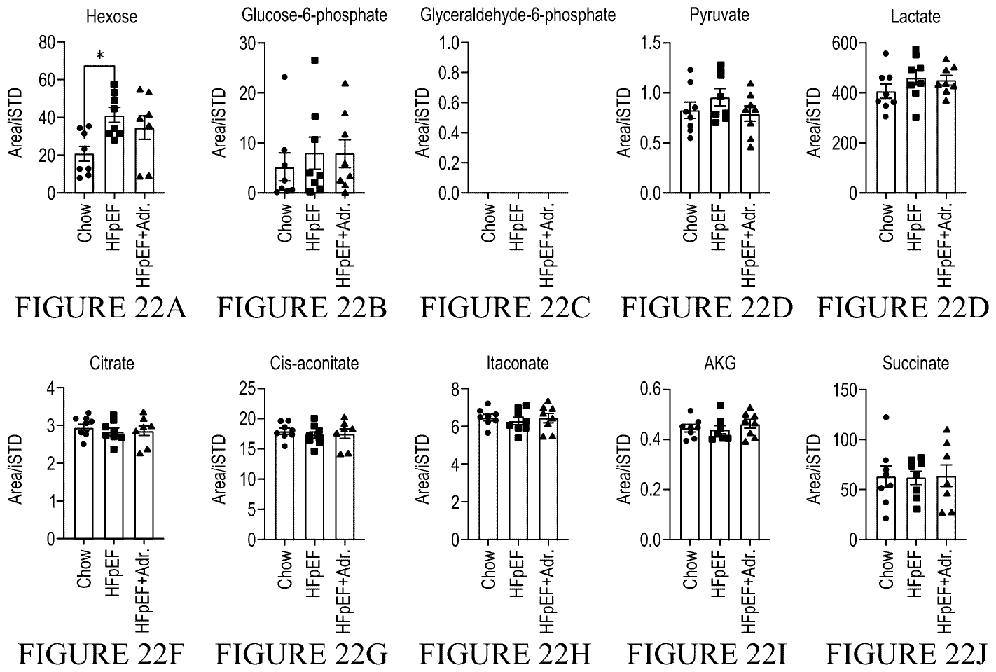

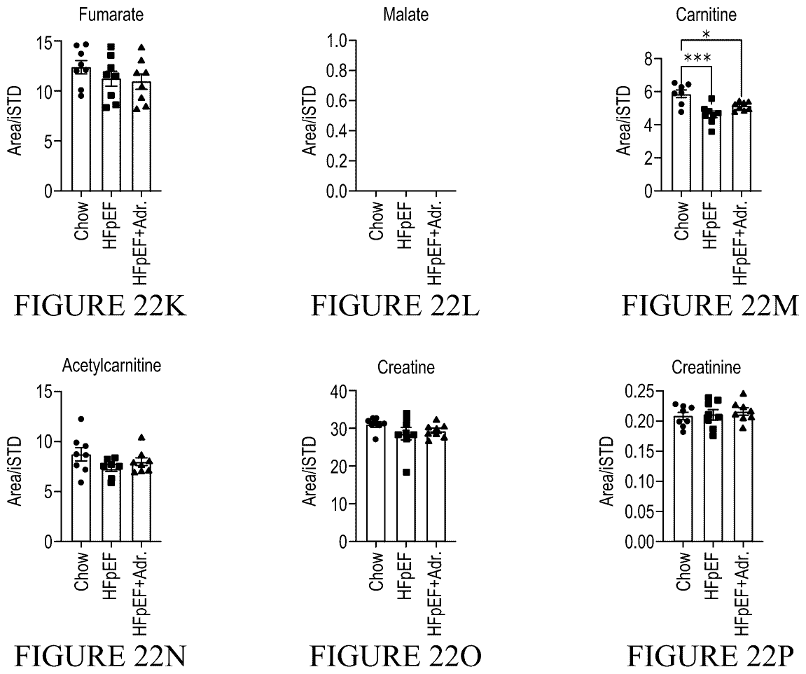

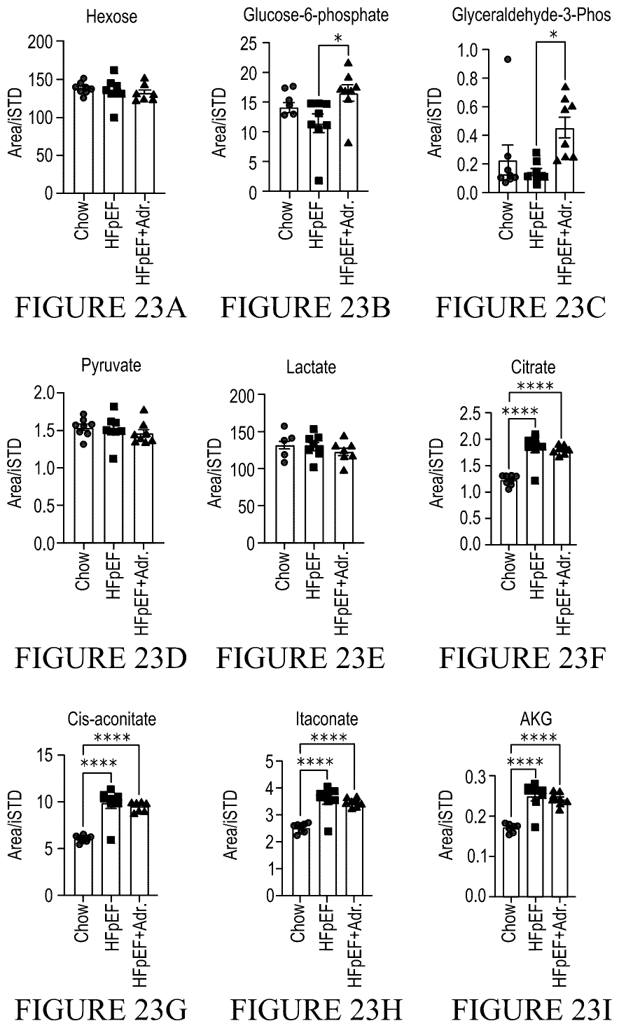

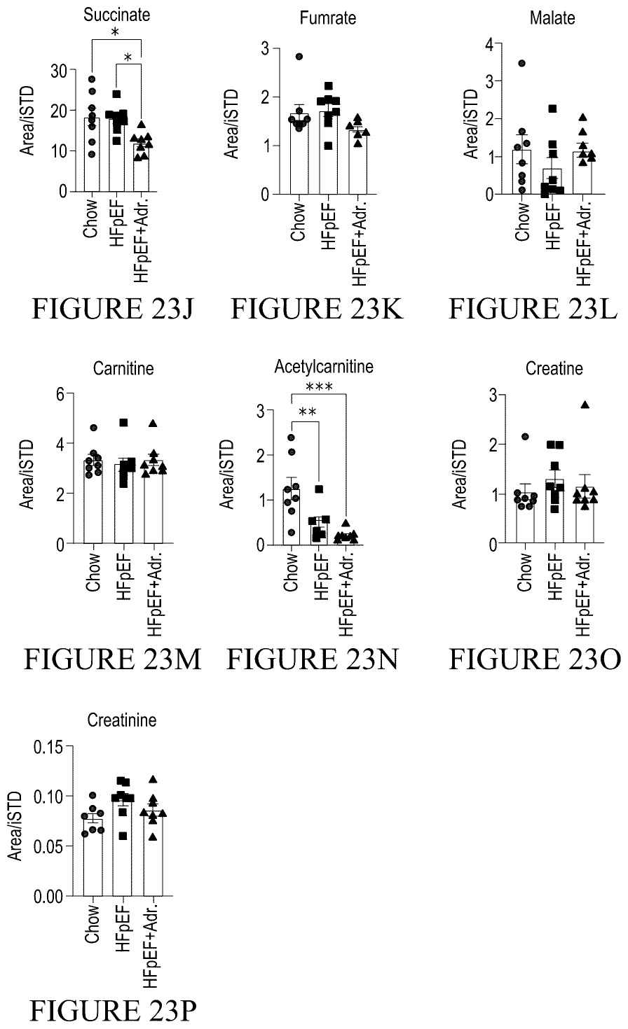

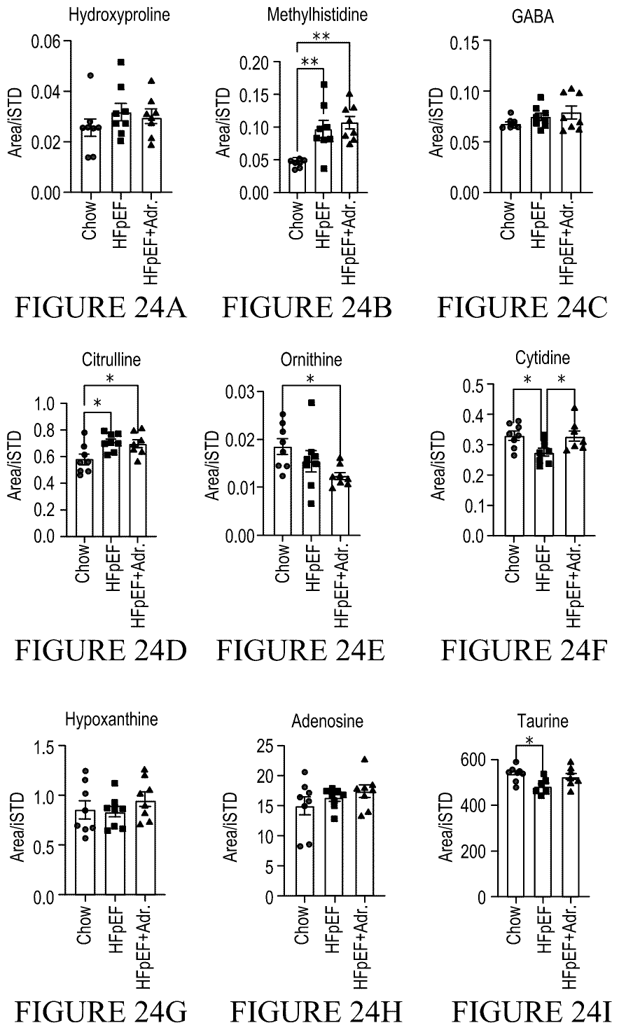

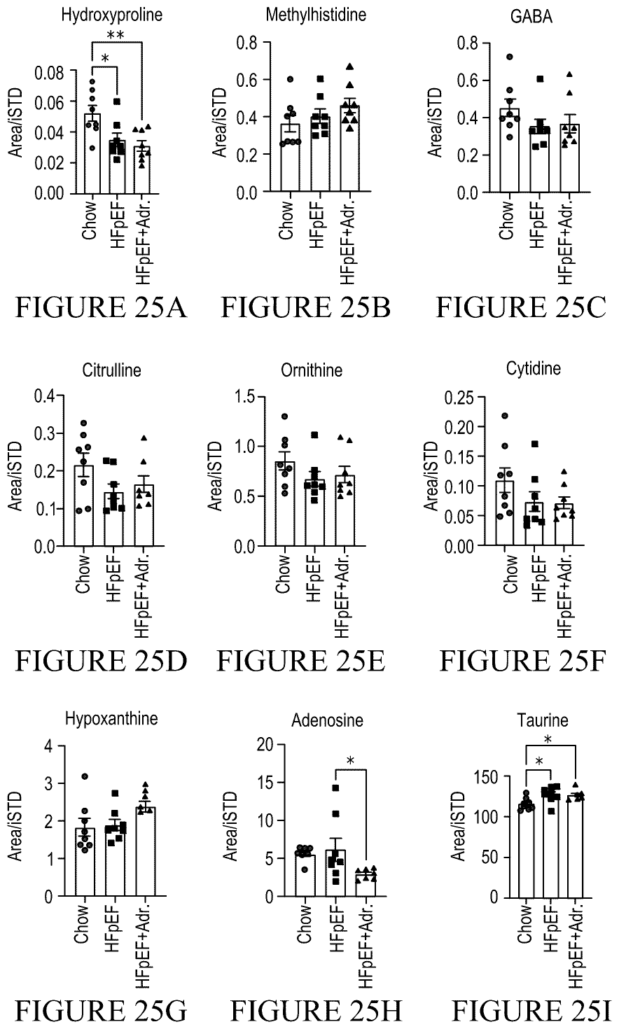

Male C57BL/6J mice were exposed to the same HFpEF model (high fat diet+L-NAME) as in FIGS. 13A-13V for 8 weeks, and subsequently received daily intraperitoneal injections of either vehicle or adropin (FIG. 15B). After one week of dosing, adropin treatment induced a non-significant decrease in body weight compared to the vehicle-treated HFpEF group, that was sustained for the duration of the study (FIG. 15C-15D). IPGTT analysis demonstrated a significant increase in circulating blood glucose levels in HFpEF mice compared to chow controls, which was significantly attenuated after adropin treatment (FIG. 15E-15F). This suggests that long-term adropin treatment inhibits hyperglycemia by improving insulin sensitivity in HFpEF mice. Echocardiographic evaluation (FIG. 15G) confirmed the development of cardiac diastolic dysfunction in HFpEF animals, as evidenced by increased LV mass (FIGS. 19A-19H), decreased cardiac output (FIG. 15H), increased isovolumetric relaxation time (IVRT; FIG. 15J), and increased left ventricular myocardial performance index (LV MPI; FIG. 15K), in the absence of systolic dysfunction (FIGS. 15I, 19A-19H). Each of these markers of diastolic dysfunction were absent in HFpEF mice treated with adropin (FIGS. 15H-15K), suggesting restored cardiac diastolic function.

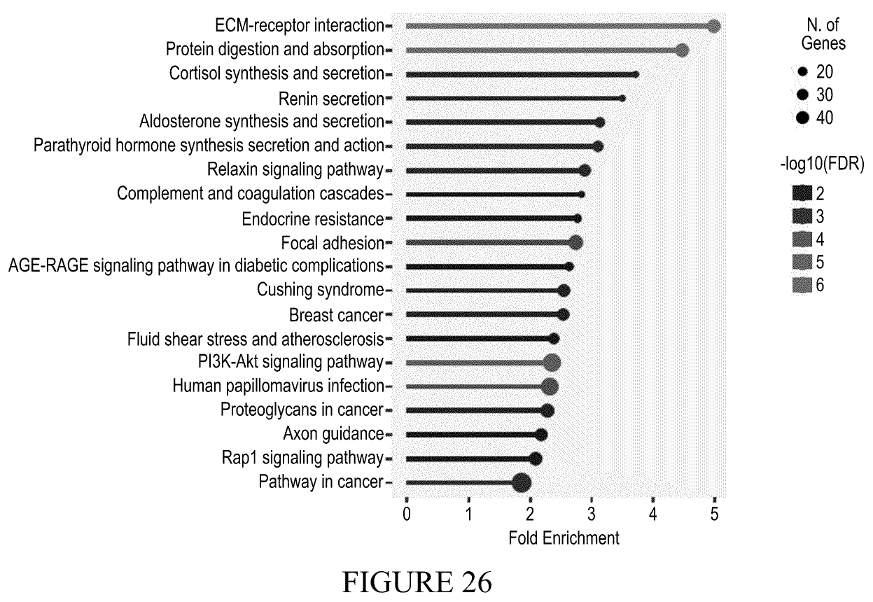

Adropin treatment attenuates left ventricular structural remodeling without affecting liver injury in HFpEF. While ex vivo studies have demonstrated the positive effects of adropin treatment on contractile function [32, 33], its impact on cardiac morphology in vivo has yet to be investigated. HFpEF mice exhibited a significant increase in normalized heart weight to tibia length when compared to chow mice, which was absent in the adropin-treated animals (FIG. 15M). Histological analyses revealed the development of significant fibrosis and cardiomyocyte hypertrophy in HFpEF mice, which were reversed upon adropin treatment (FIG. 15L-150). Bulk RNA-seq analysis revealed that several extracellular matrix remodeling transcripts, including Col1a1, Col3a1, Col5a1, and Mmp2, had a >2-fold decrease in adropin-treated mice compared to vehicle-treated HFpEF mice (FIG. 26). These transcriptomics data provide further evidence that adropin treatment mitigates cardiac fibrosis as observed in histological analyses.

Recent studies have shown that exogenous adropin treatment alleviates hepatocellular injury in NAFLD/MAFLD [34]. Therefore, it was next assessed whether adropin-mediated protective effects in the liver are sustained in HFpEF. As demonstrated above (FIG. 13N-13Q), HFpEF mice developed NAFLD/MAFLD as shown by increased steatosis, ballooning (P=0.09), and inflammation (P=0.08); however, adropin treatment led to only a non-significant decrease (˜7%) in liver NAS (FIG. 15P-15T). Together, these findings show that prolonged adropin treatment ameliorates cardiac structural abnormalities and maladaptive remodeling.

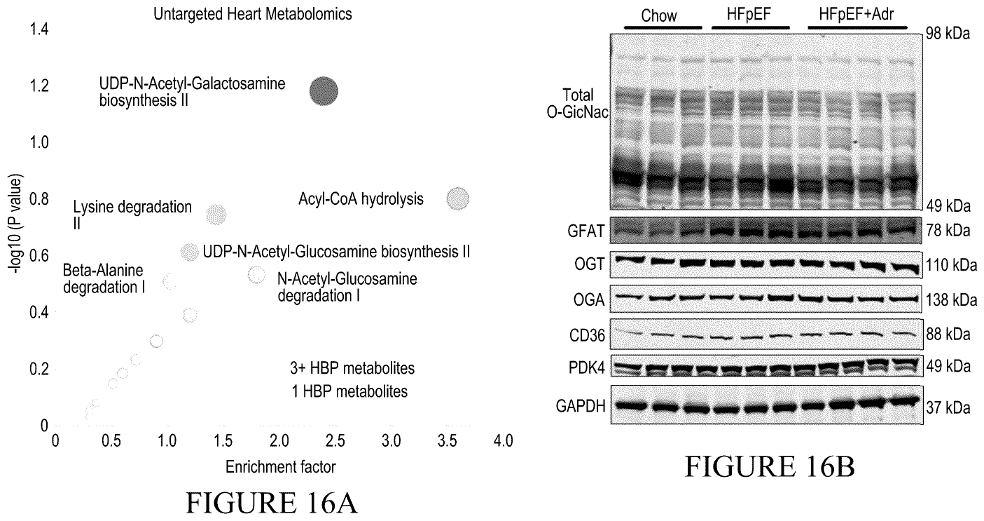

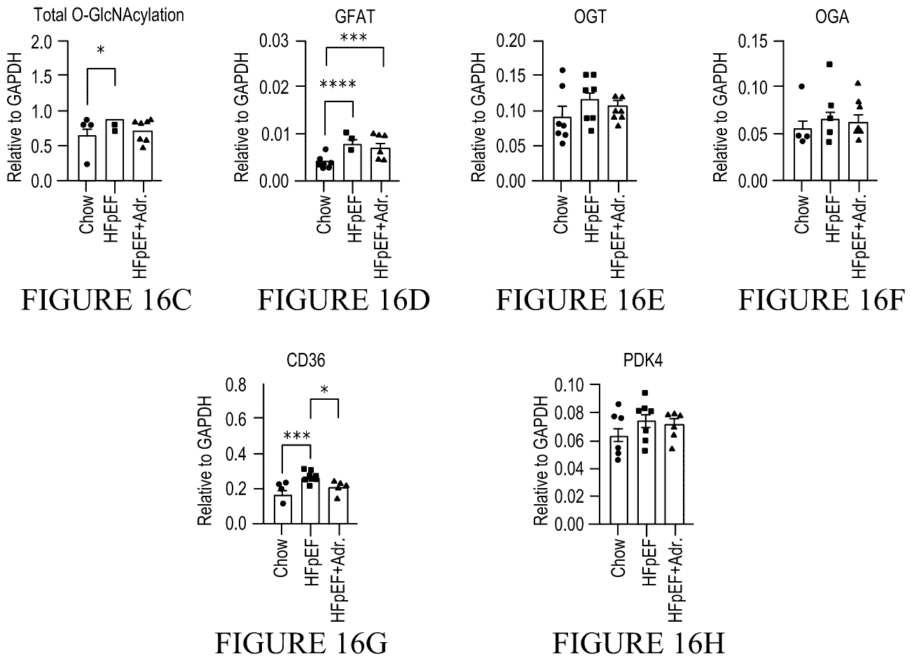

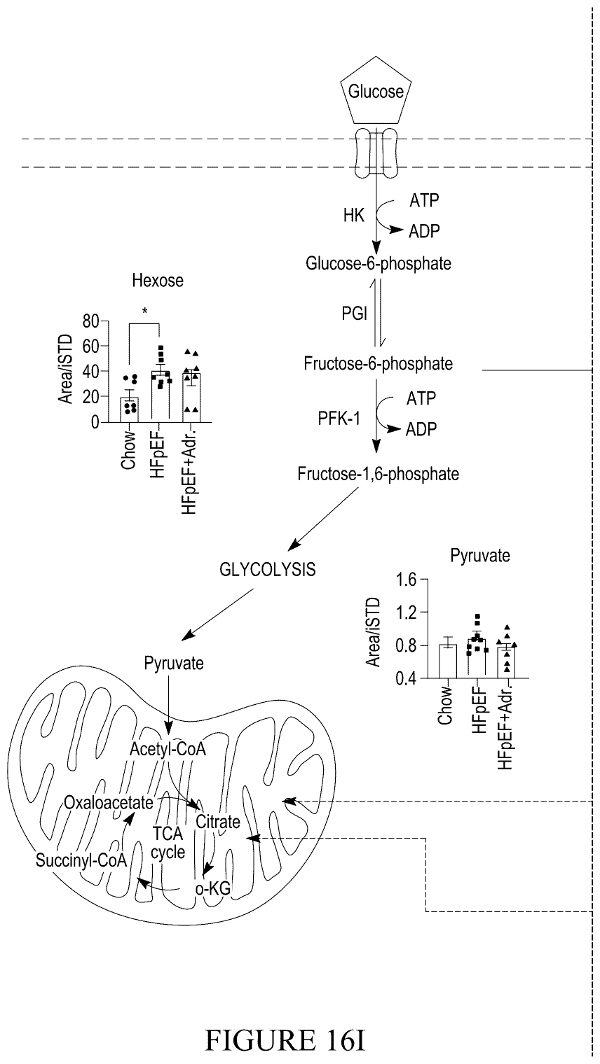

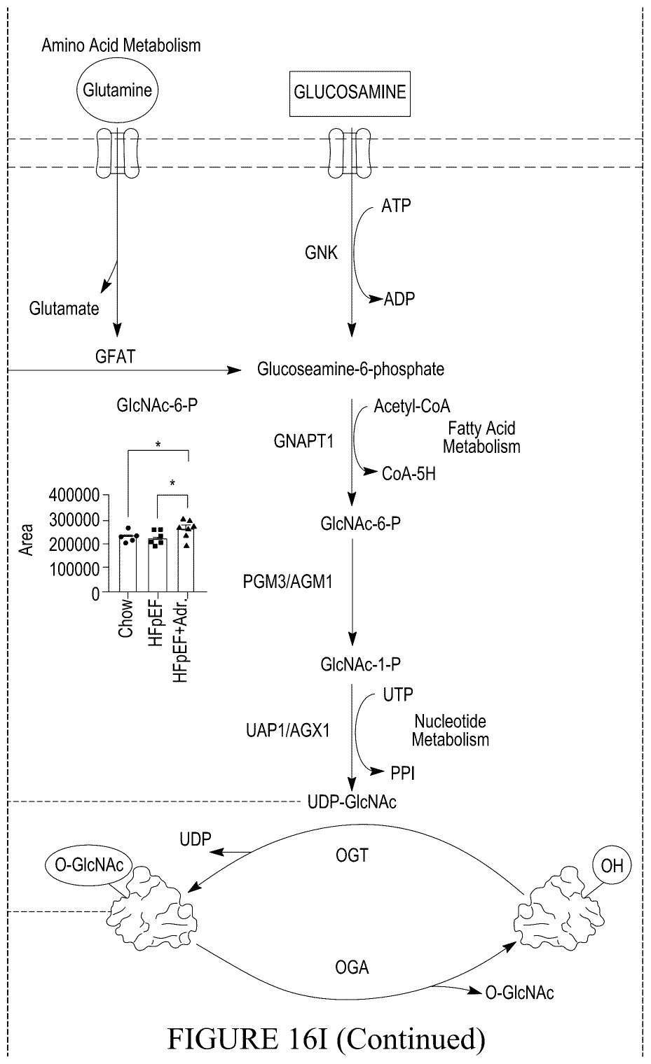

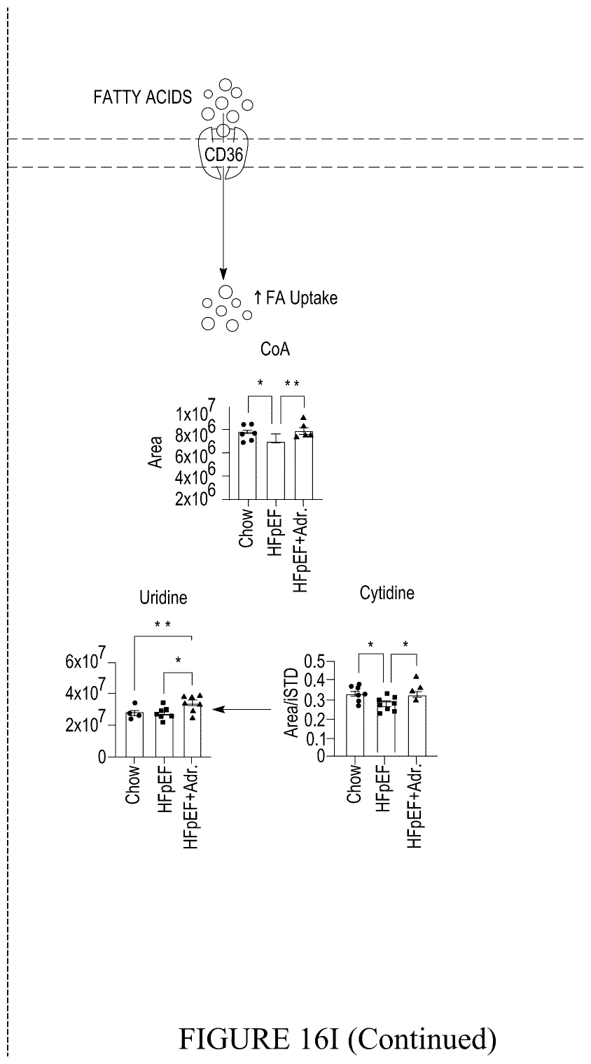

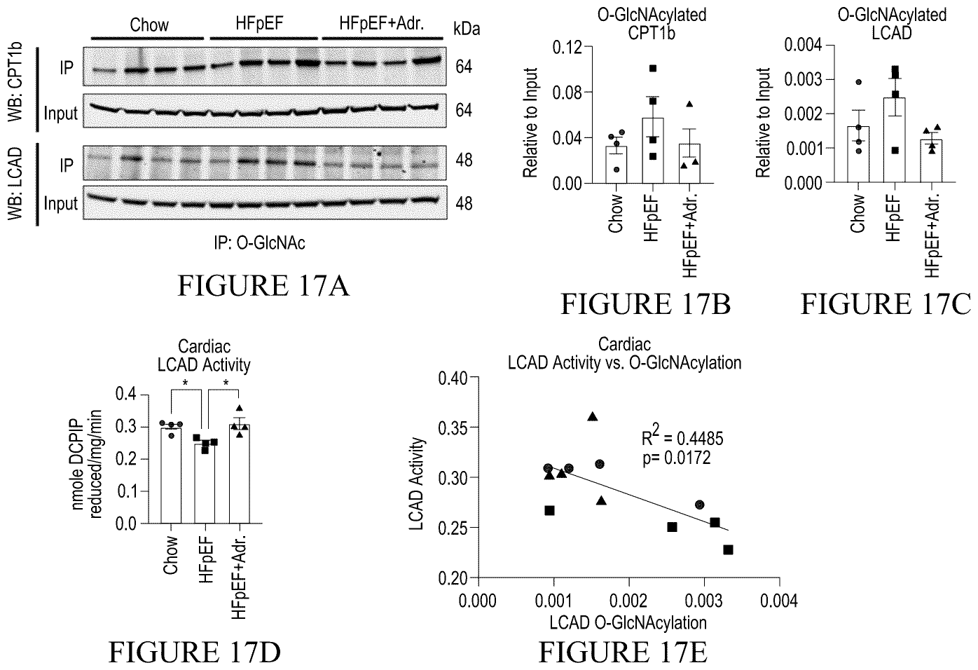

Adropin treatment inhibits flux into the hexosamine biosynthesis pathway (HBP) in HFpEF. Short-term adropin treatment has been shown to drive cardiac fuel utilization towards glucose oxidation, through increased pyruvate dehydrogenase activity [30] and augmented insulin signaling [32]. However, its long-term effect on metabolic flexibility has yet to be evaluated. Therefore, it was next determined whether the adropin-mediated improvements in cardiac function and structure were linked to changes in energy substrate utilization. Untargeted BioCyc pathway analysis revealed that metabolites related to UDP-N-acetyl-D-glucosamine (UDP-GlcNAc) biosynthesis represented the major metabolomic difference between vehicle-treated and adropin-treated HFpEF groups (FIG. 16A), thereby implicating the hexosamine biosynthesis pathway (HBP) modulation as a potential mechanism underlying adropin protection in HFpEF.

The HBP is a nutrient sensing signaling pathway that coordinates flux through major energy metabolism pathways, integrating glucose, amino acid, fatty acid, and nucleotide metabolism to generate UDP-GlcNAc, the donor substrate for protein 0-GlcNAcylation. Targeted metabolomics and biochemical analyses suggest that flux through the HBP was significantly increased in HFpEF mice, as shown by increased expression of the rate-limiting HBP enzyme glutamine fructose-6-phosphateaminotransferase (GFAT; FIG. 16C), and a significant increase in total cardiac protein O-GlcNAcylation (FIG. 16F). Additionally, there was a significant increase in the abundance of the plasma fatty acid transporter CD36 in HFpEF mice (FIG. 16G), which can exacerbate HBP activity via the increased provision of fatty acid oxidation substrates for the generation of HBP intermediates (FIG. 16I). While adropin treatment did not reduce GFAT expression (FIG. 16D), it restored total protein O-GlcNAcylation (FIG. 16C; P=0.06) and CD36 abundance to levels observed in chow mice (FIG. 16G). Interestingly, there were no observed changes in PDK4 enzyme expression (FIG. 16H). Furthermore, adropin treatment did not inhibit cardiac protein 0-GlcNAcylation by mediating expression of the enzymes regulating the dynamic on and off cycling of the O-GlcNAc moiety, OGT and OGA (FIG. 16E-16F). Instead, metabolomic analysis suggested that adropin decreased substrate entry from major metabolic pathways that contributes to the HBP (FIG. 16I). Relative to HFpEF mice, adropin treatment led to a significant increase in the abundance of metabolites used to generate UDP-GlcNAc, such as coenzyme A, GlcNAc-6-P, and uridine (which is deaminated from cytidine), suggesting a decrease in their use for UDP-GlcNAc biosynthesis (FIG. 16I). Combined with a trend towards restored cardiac hexose and pyruvate levels (FIG. 16I), these data suggest that adropin treatment reduces metabolite entry into the HBP, thereby increasing the use of glycolysis intermediates for oxidative energy metabolism.

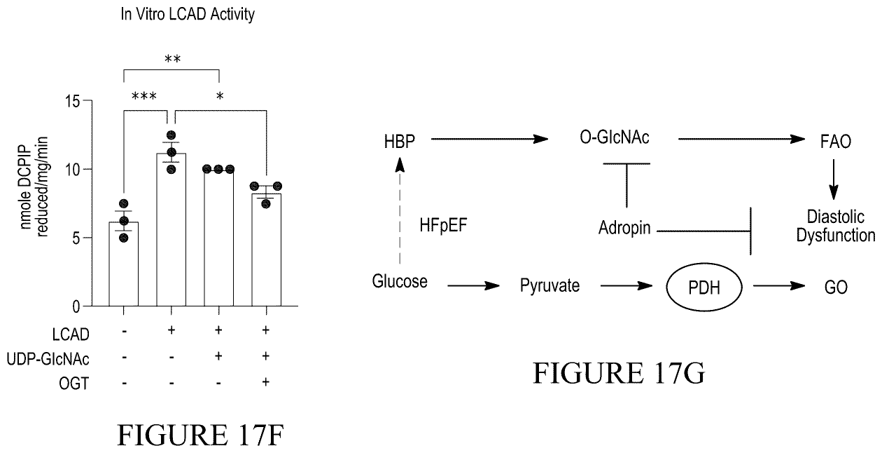

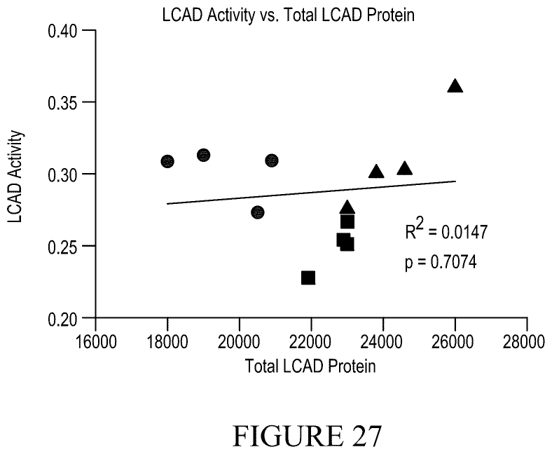

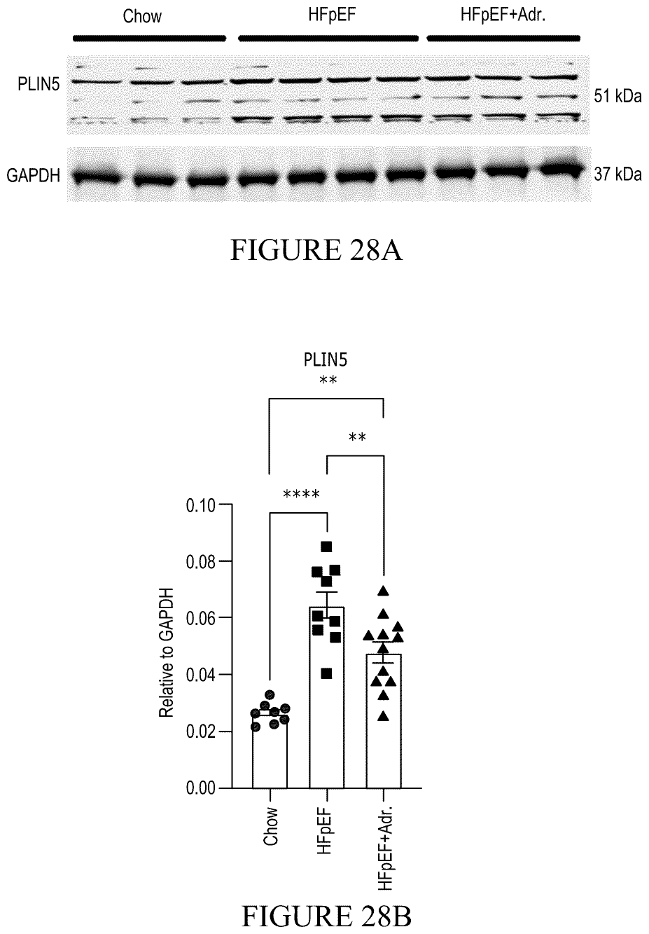

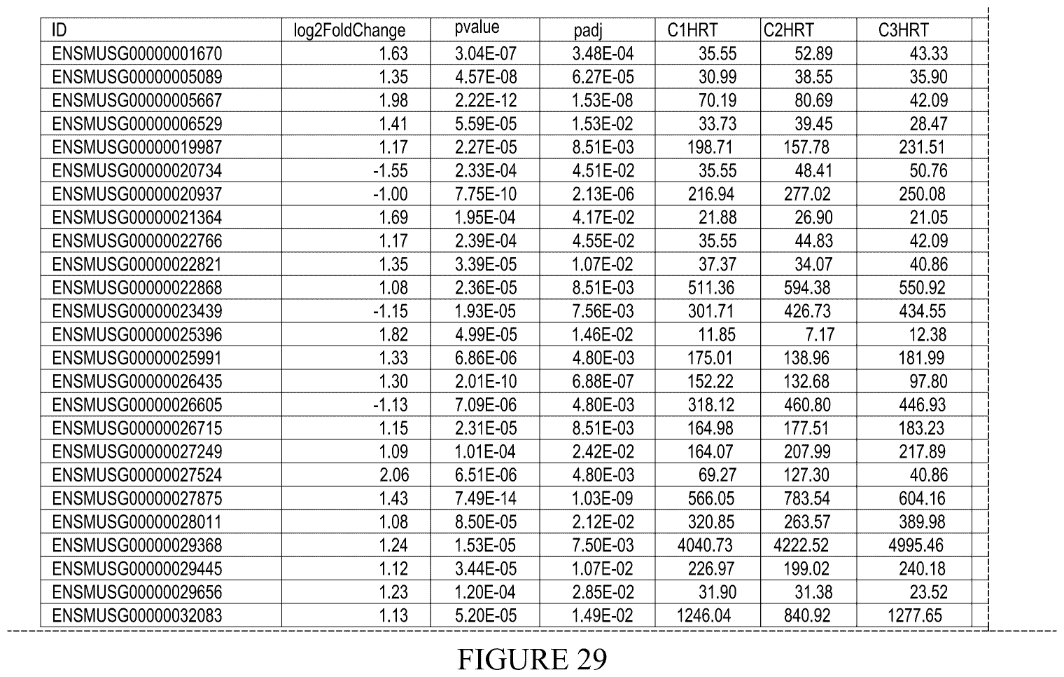

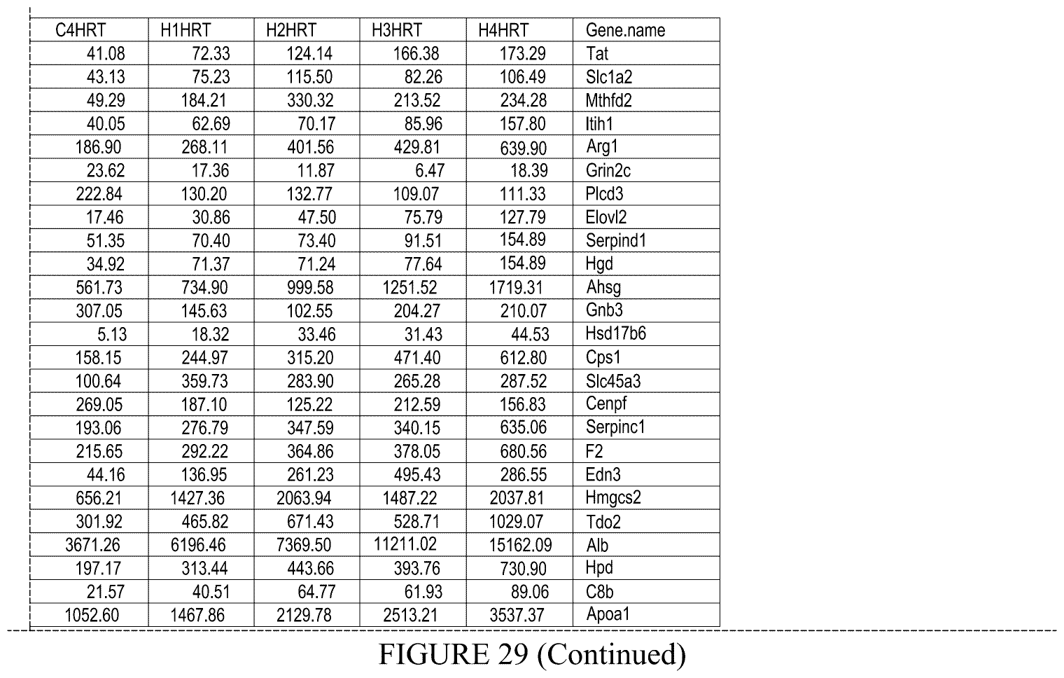

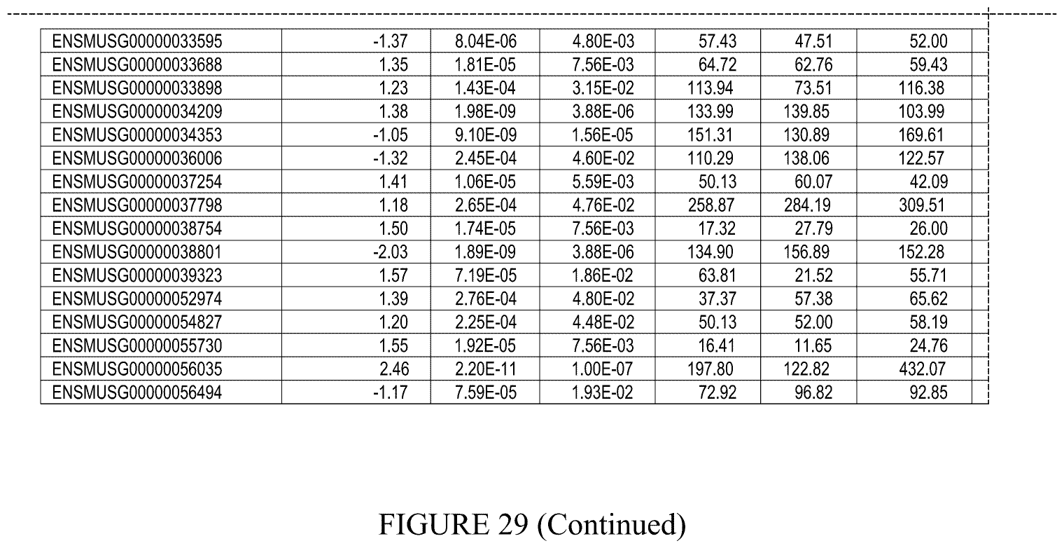

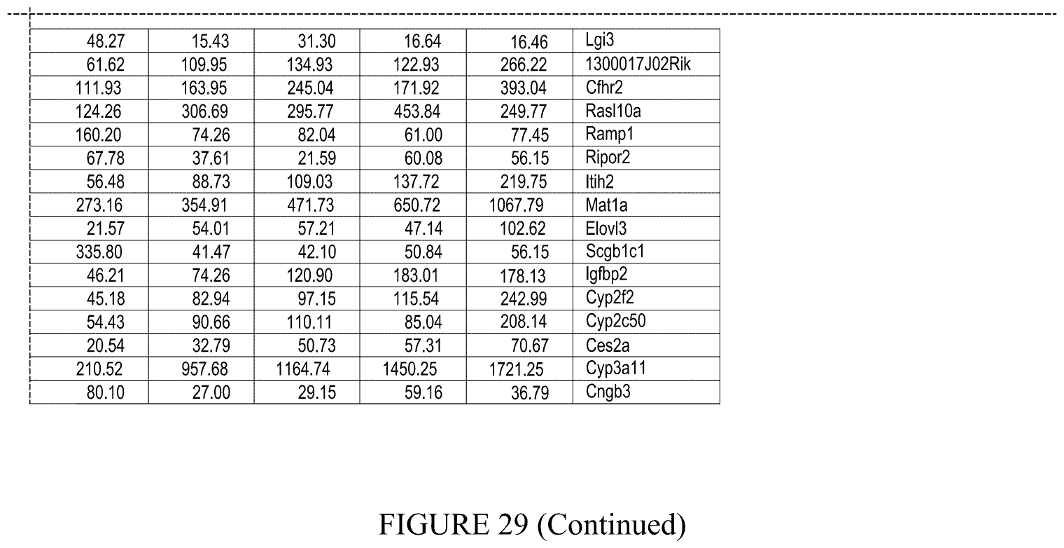

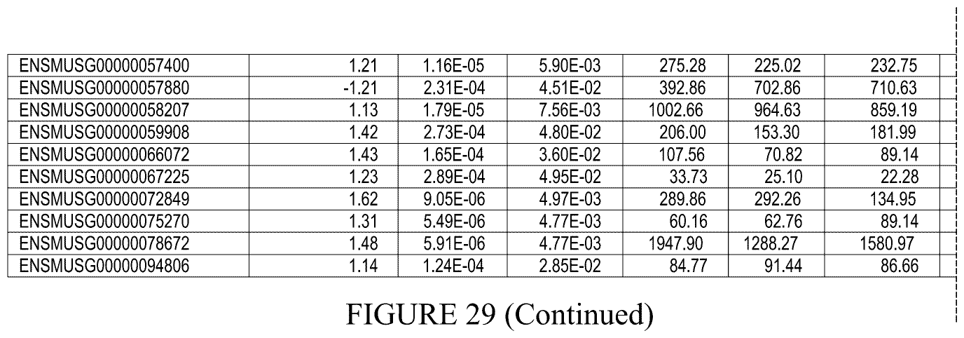

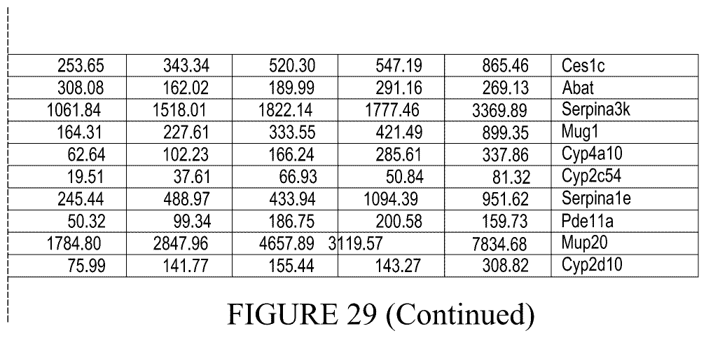

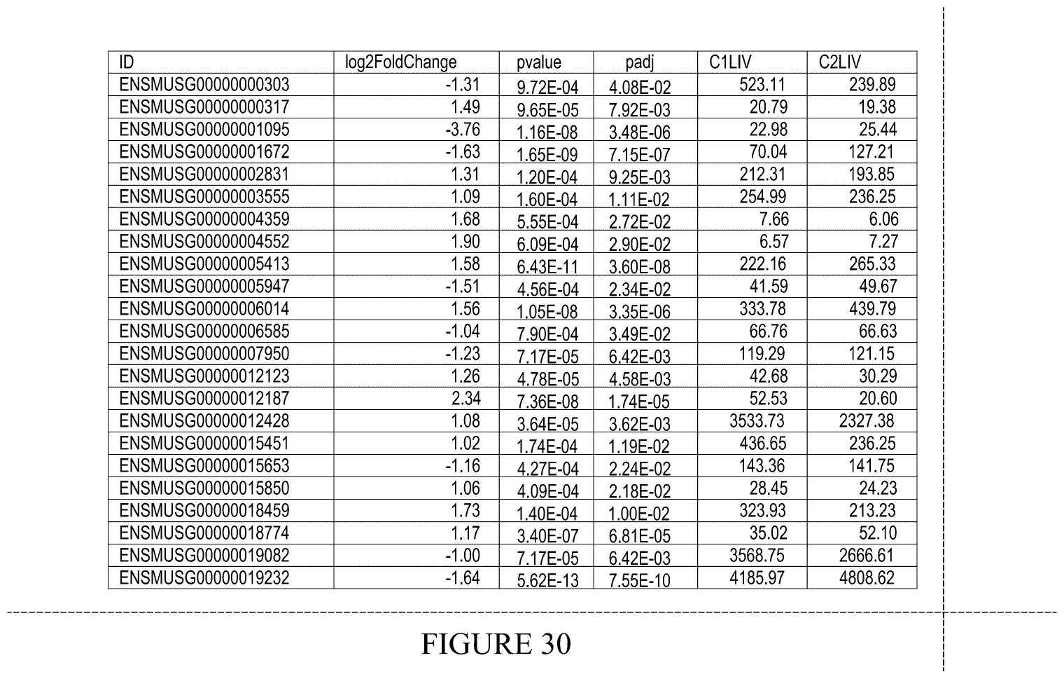

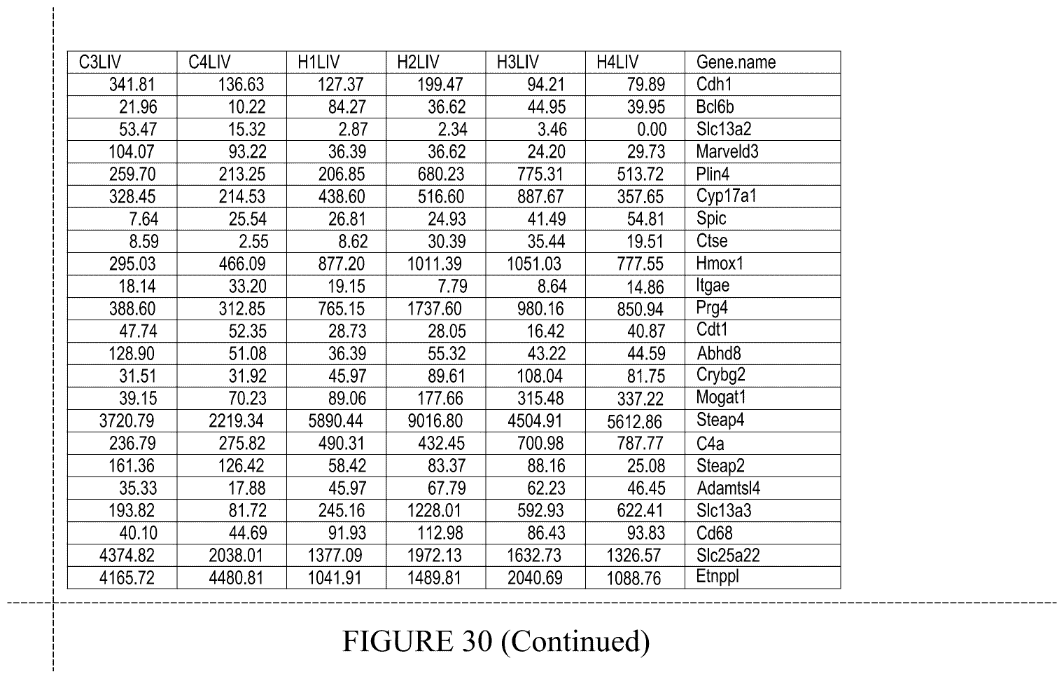

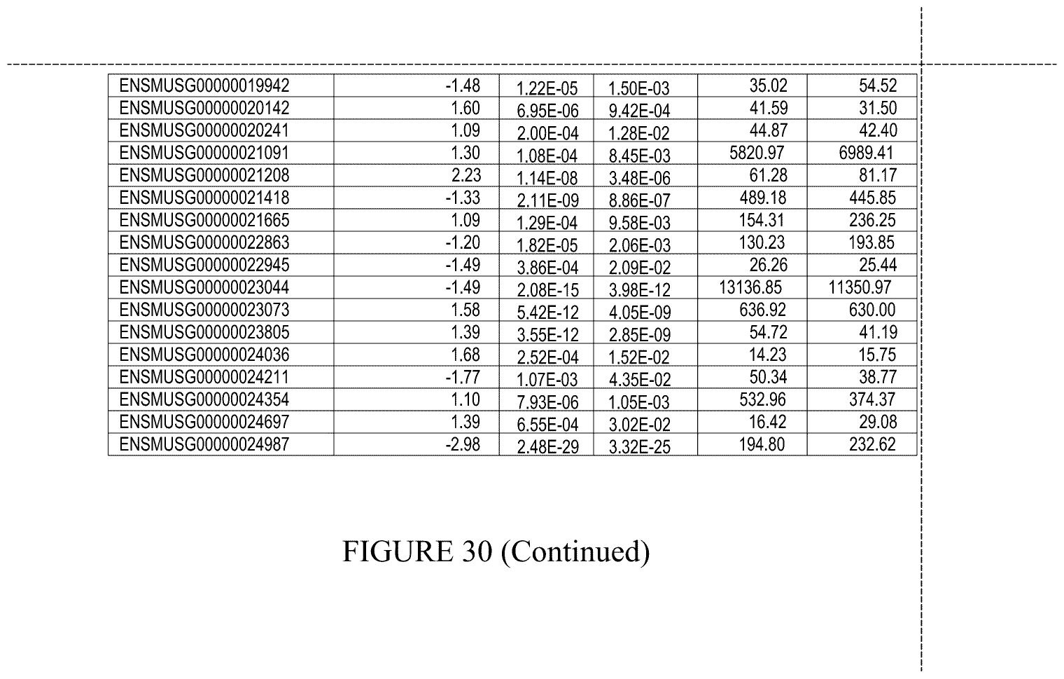

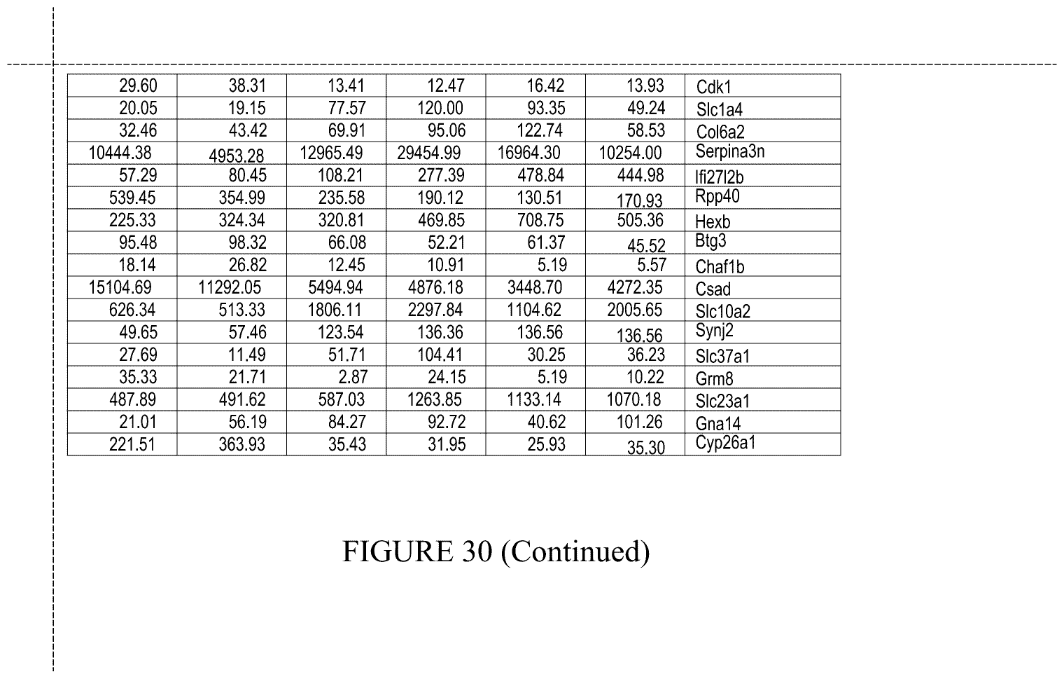

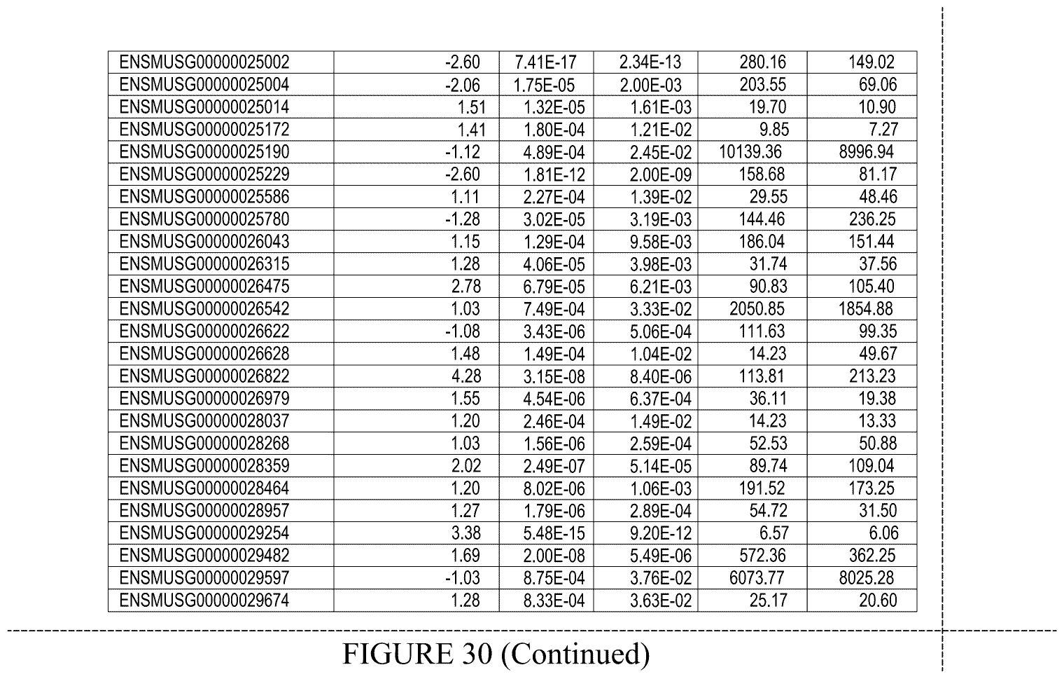

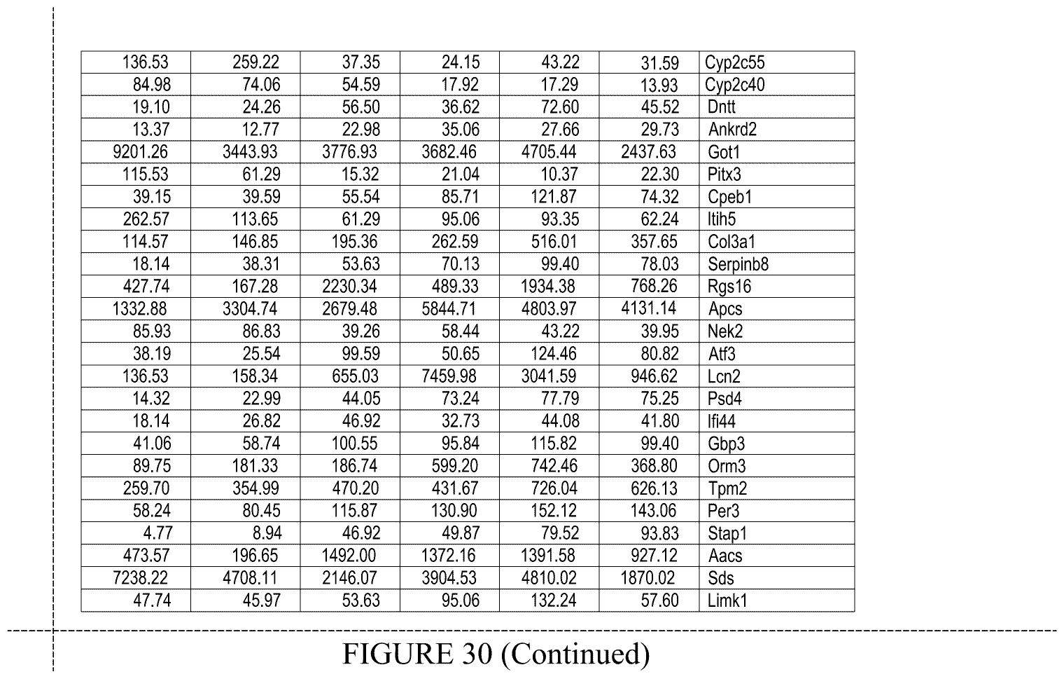

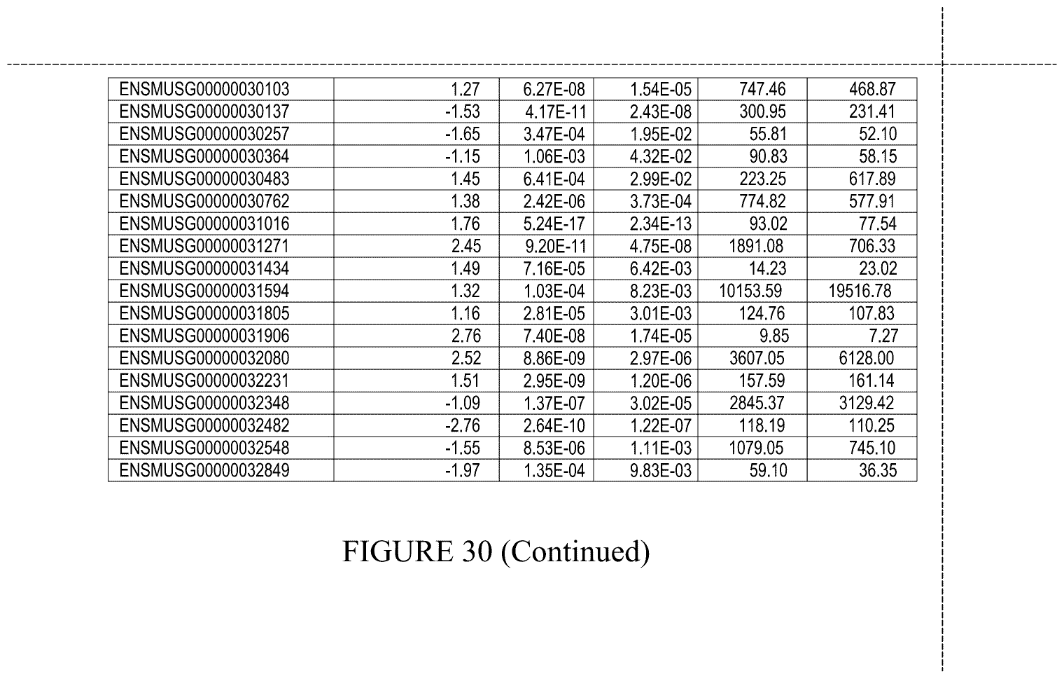

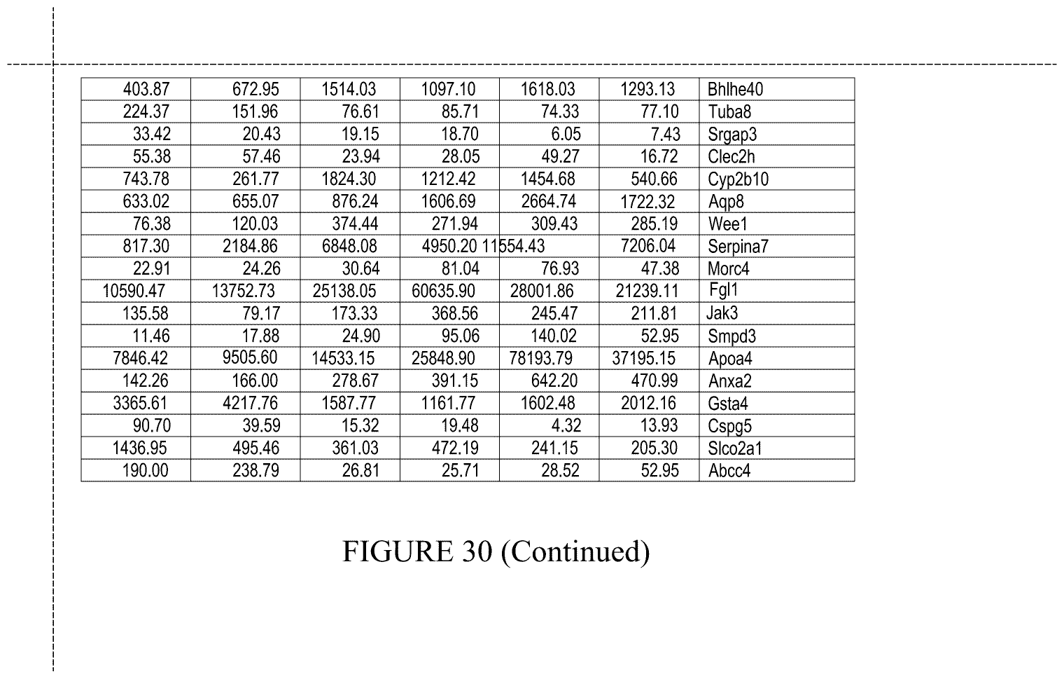

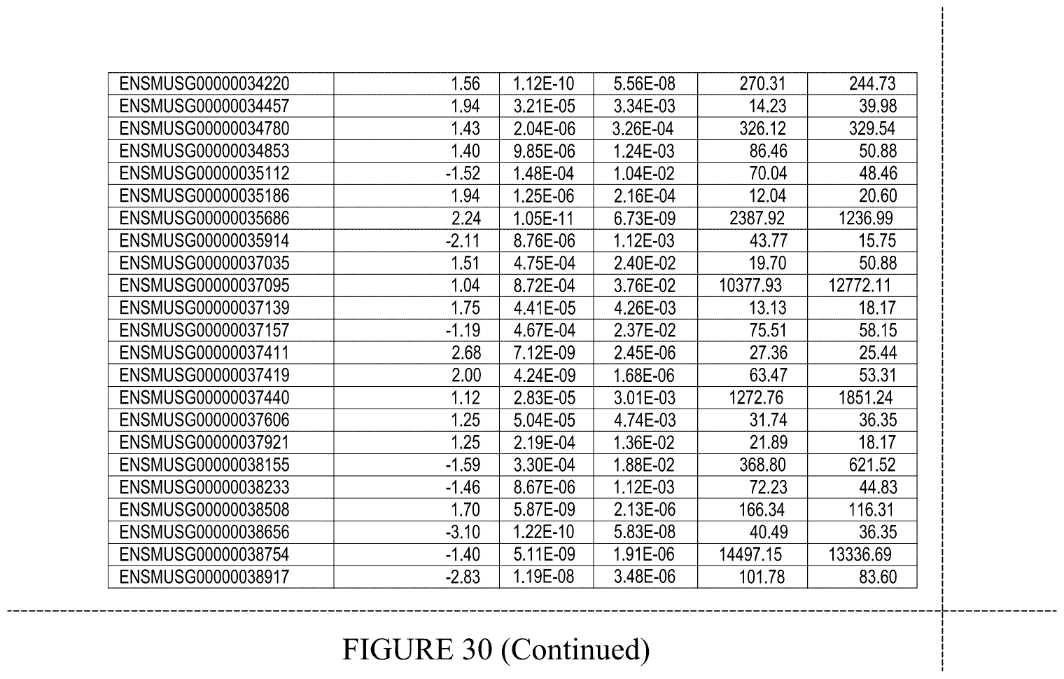

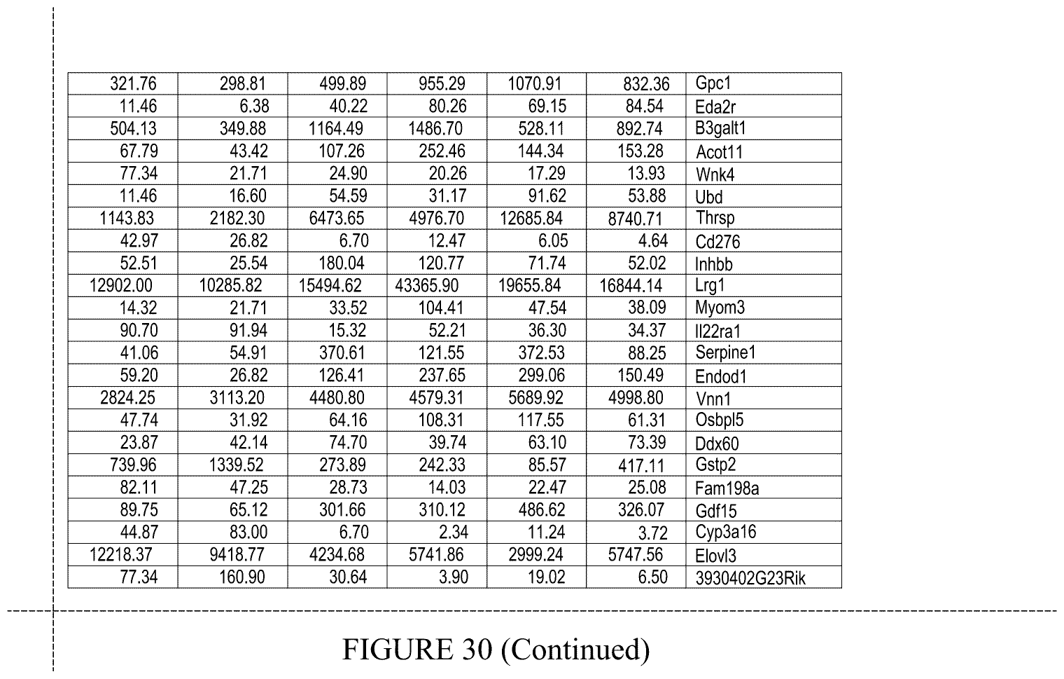

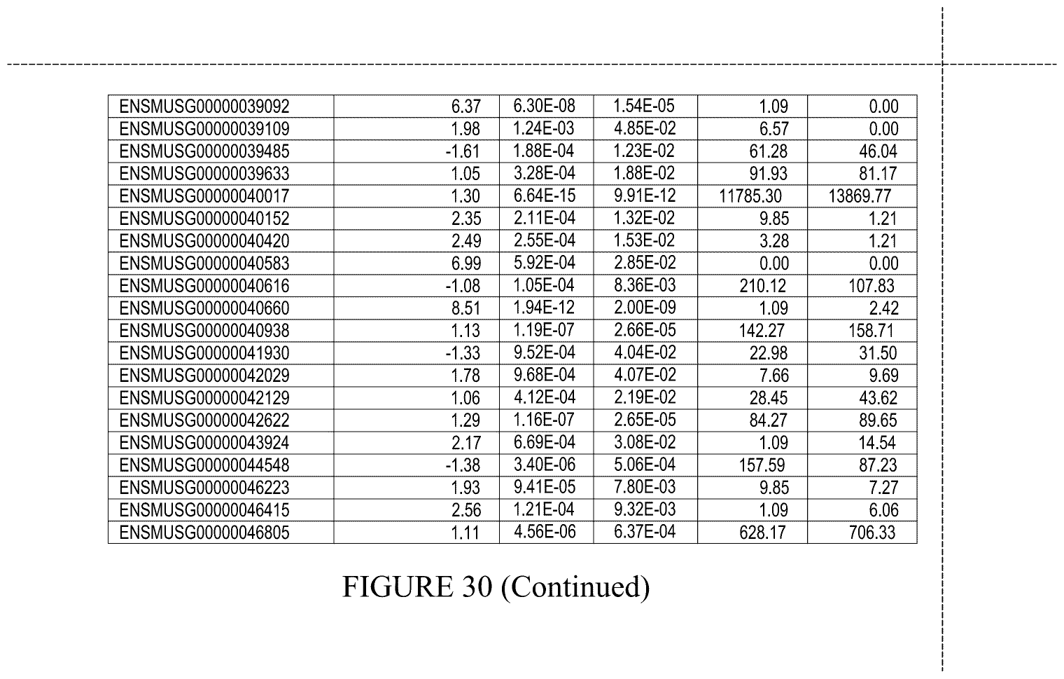

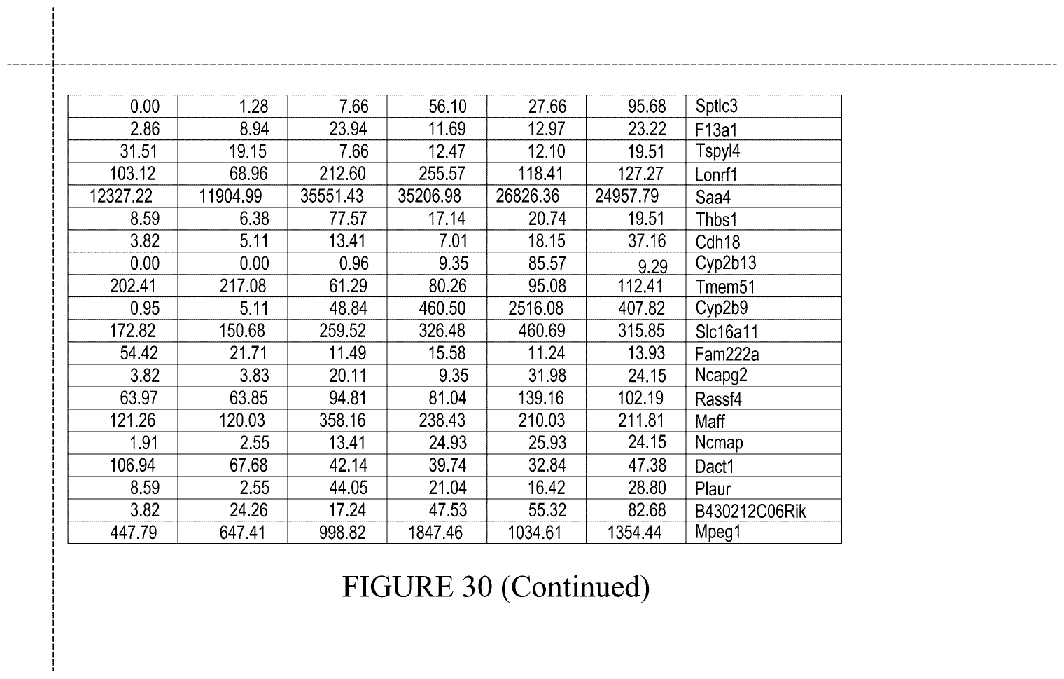

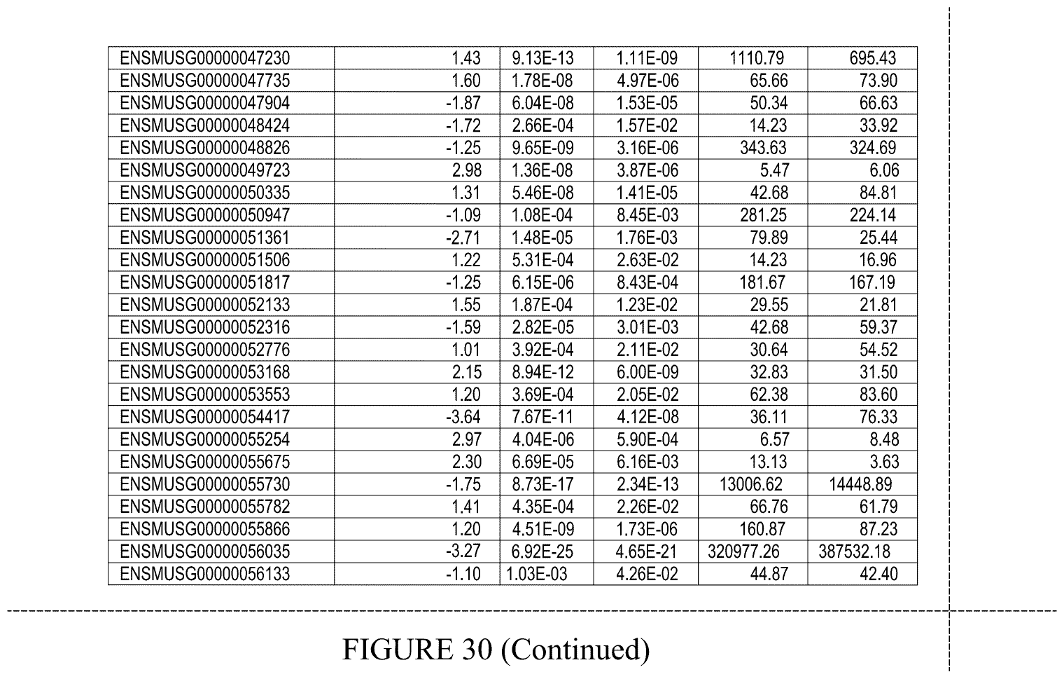

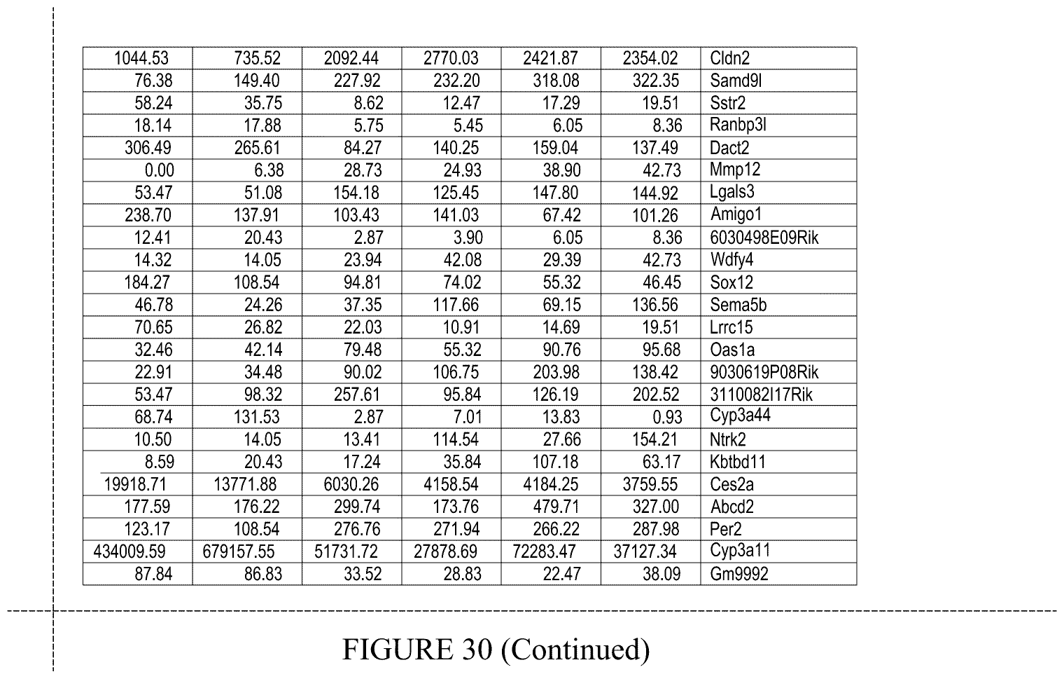

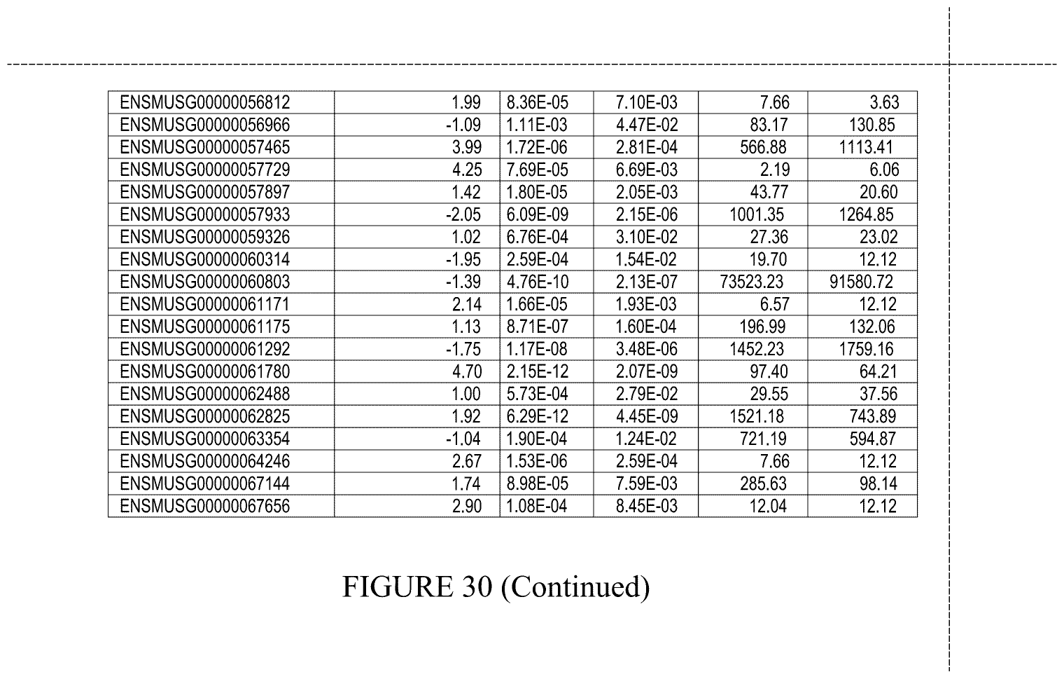

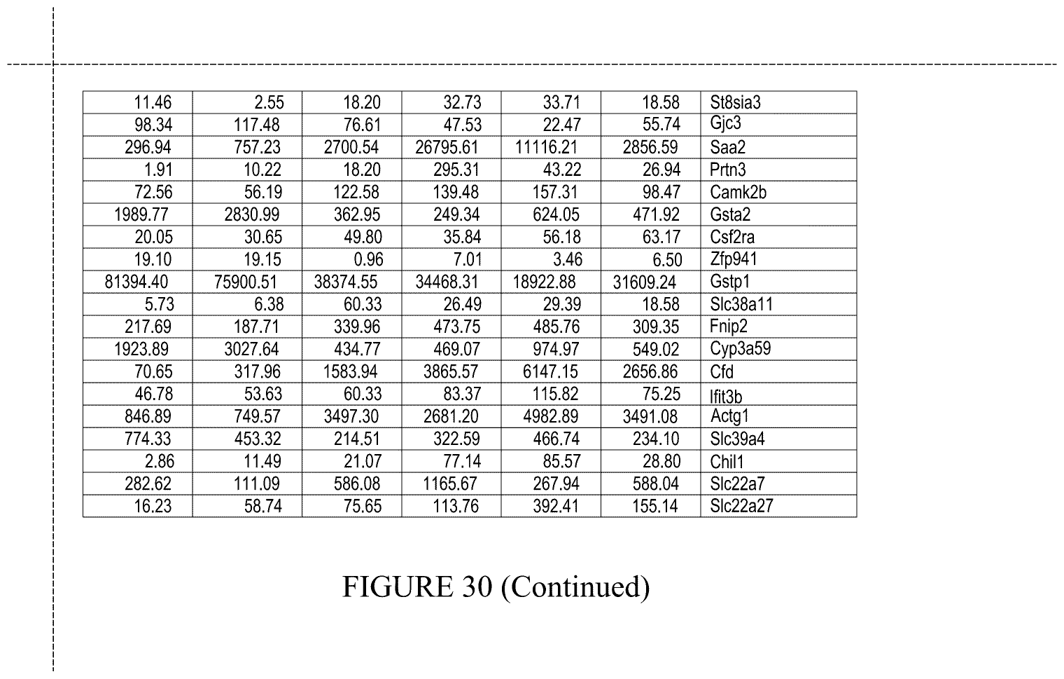

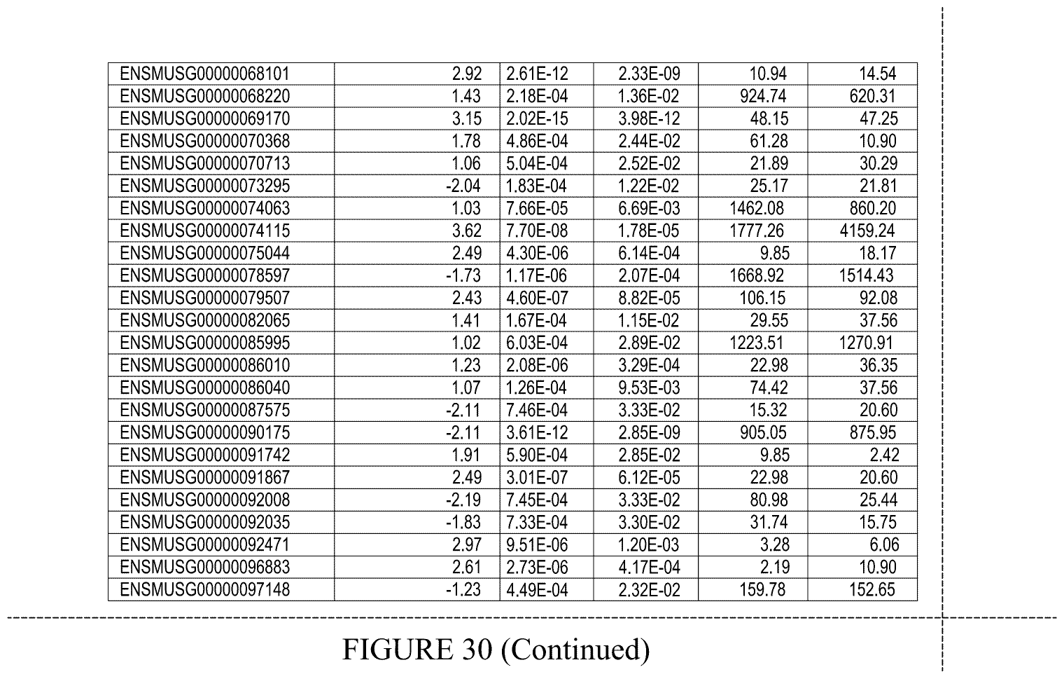

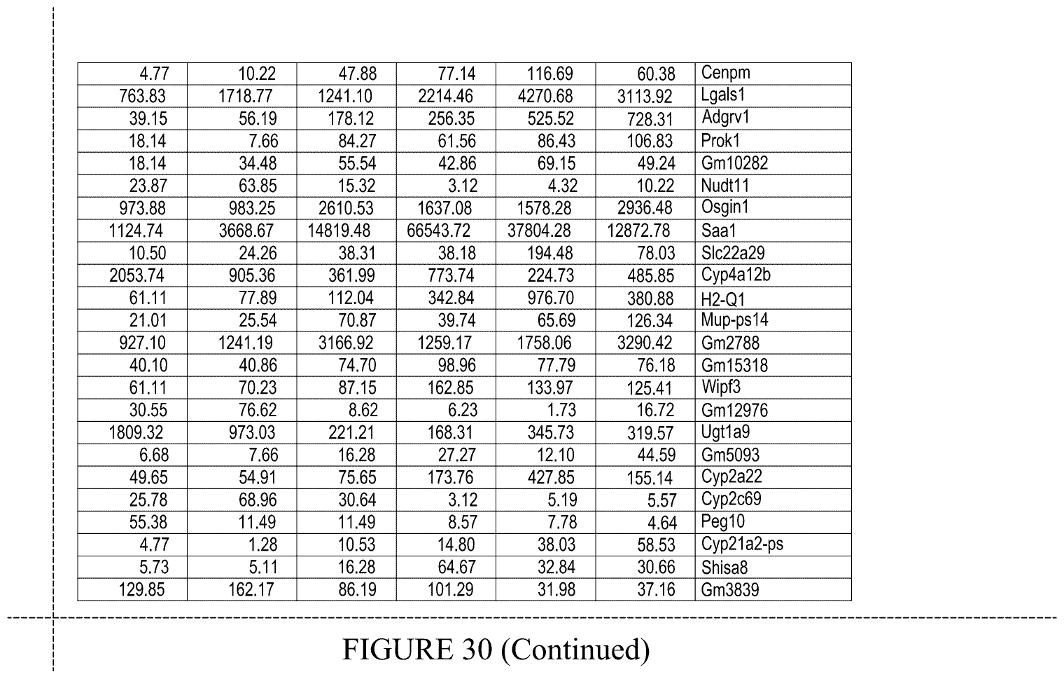

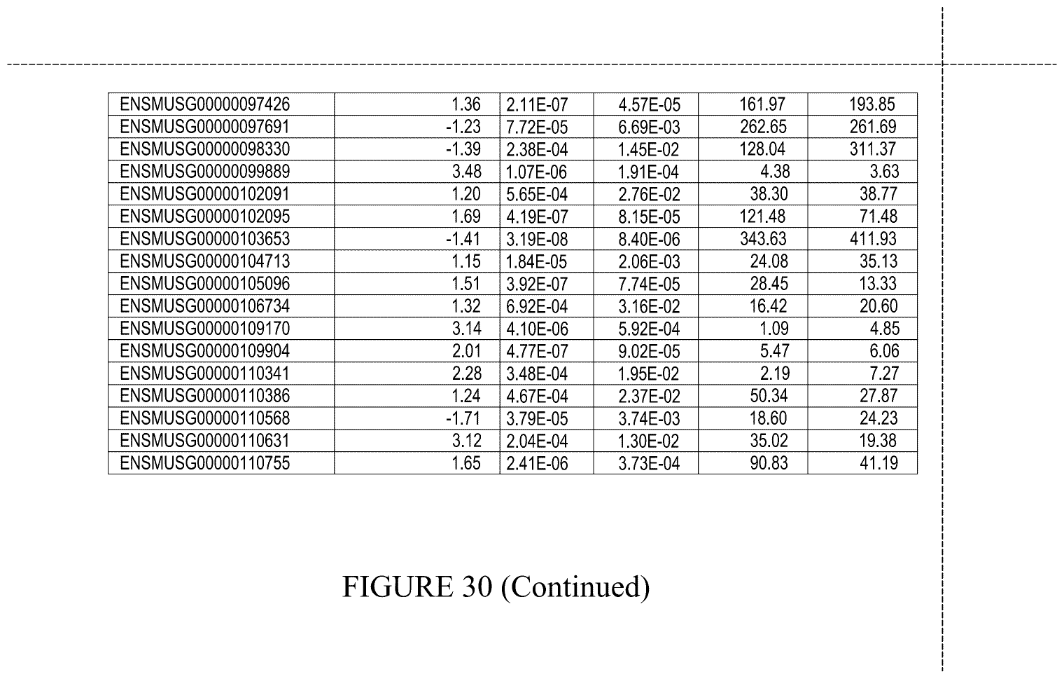

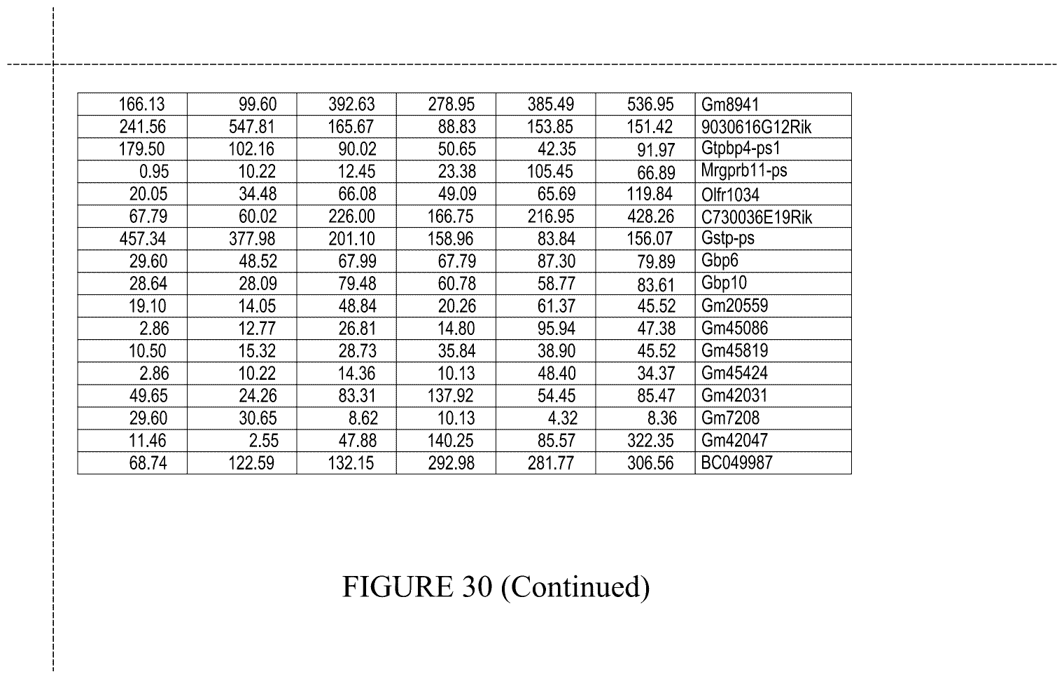

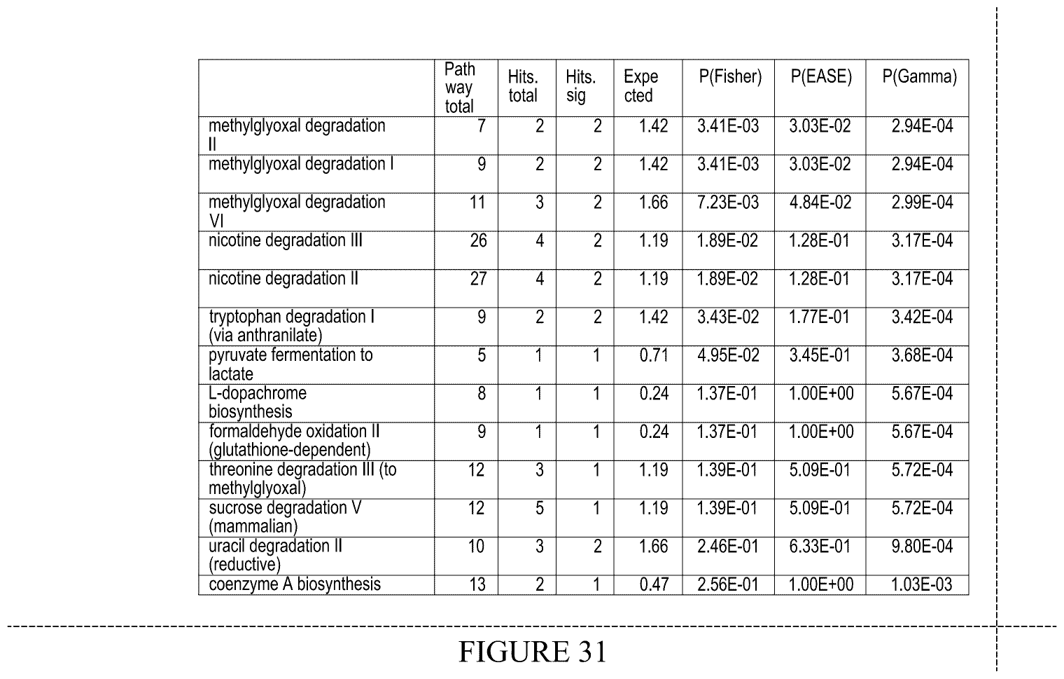

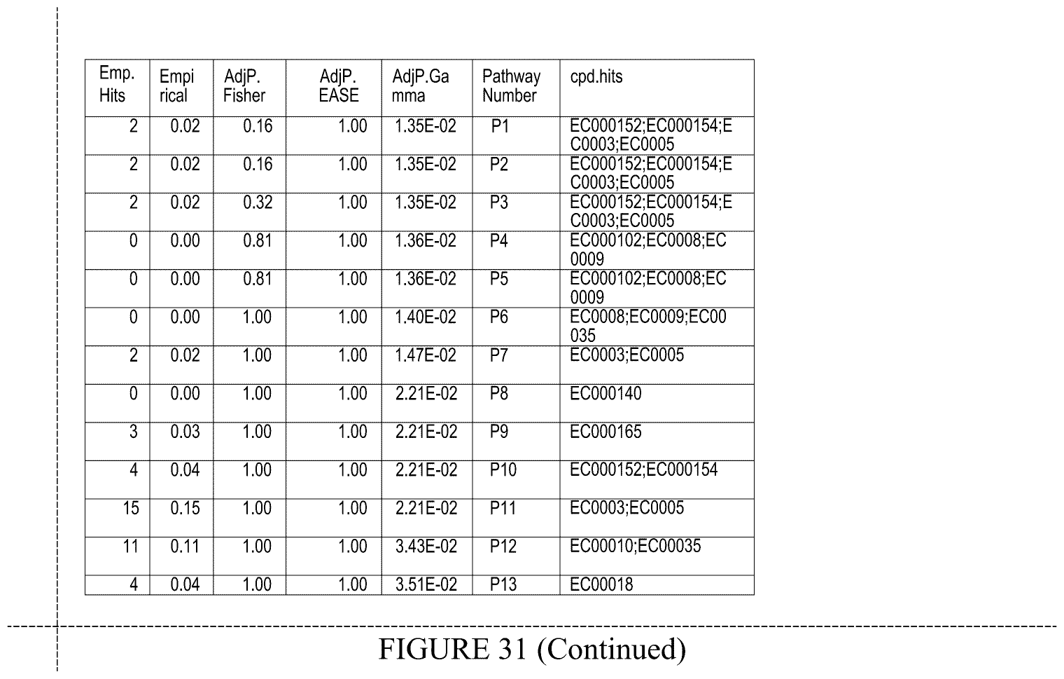

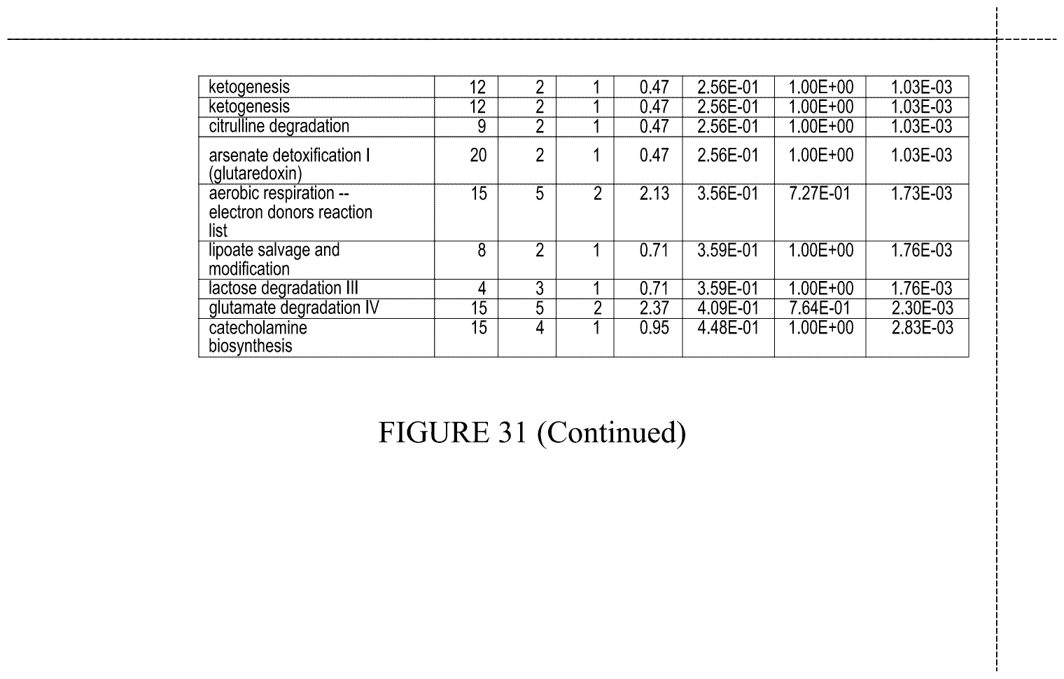

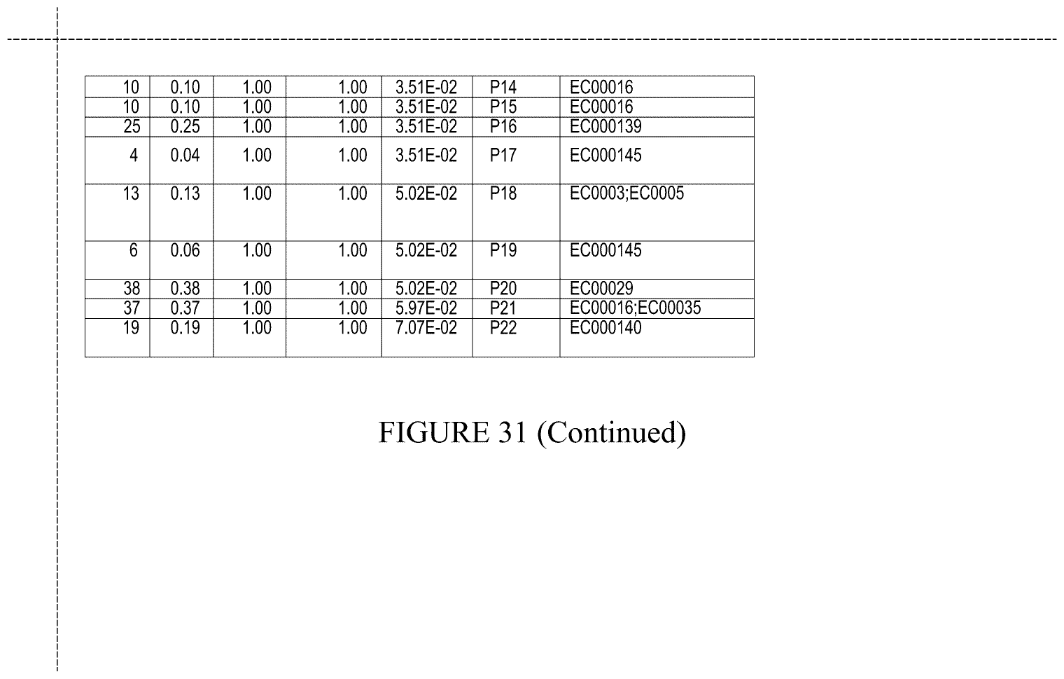

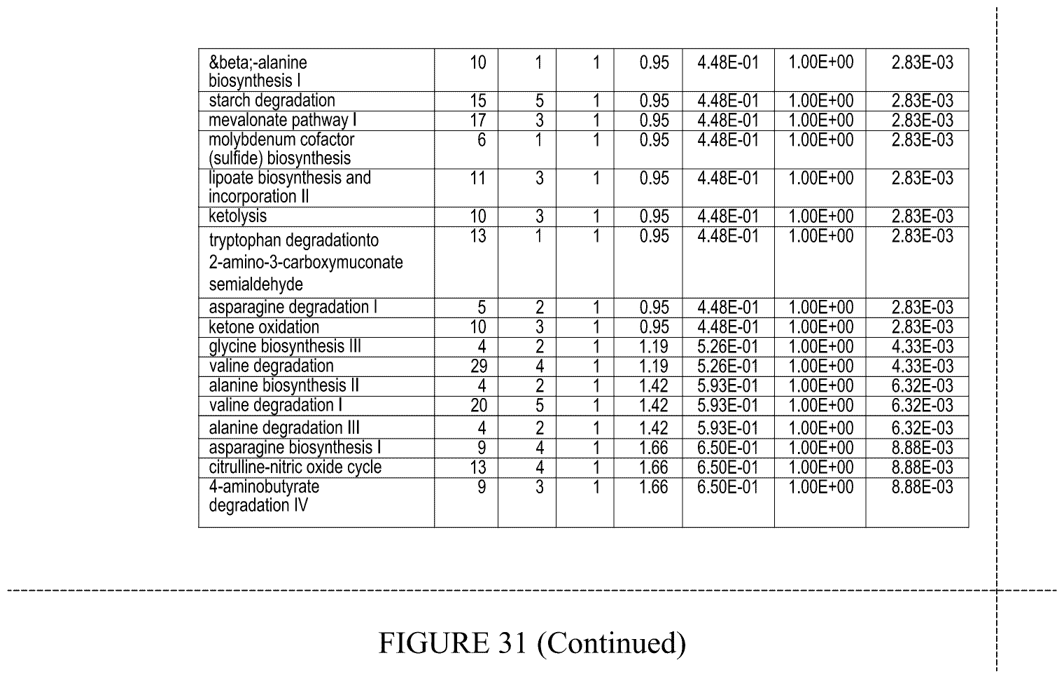

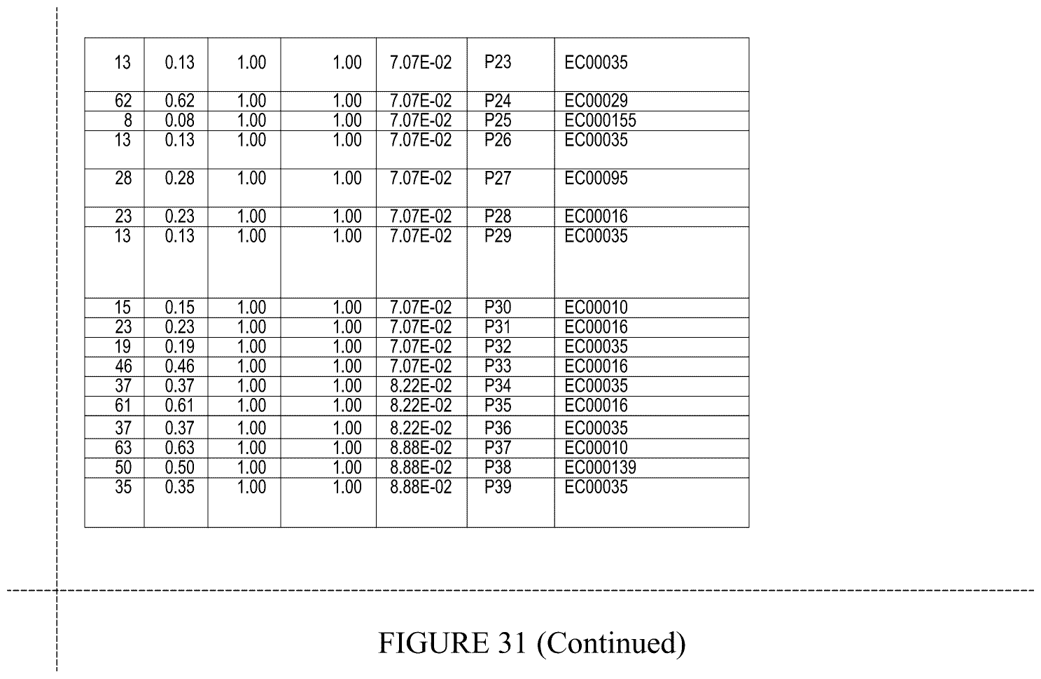

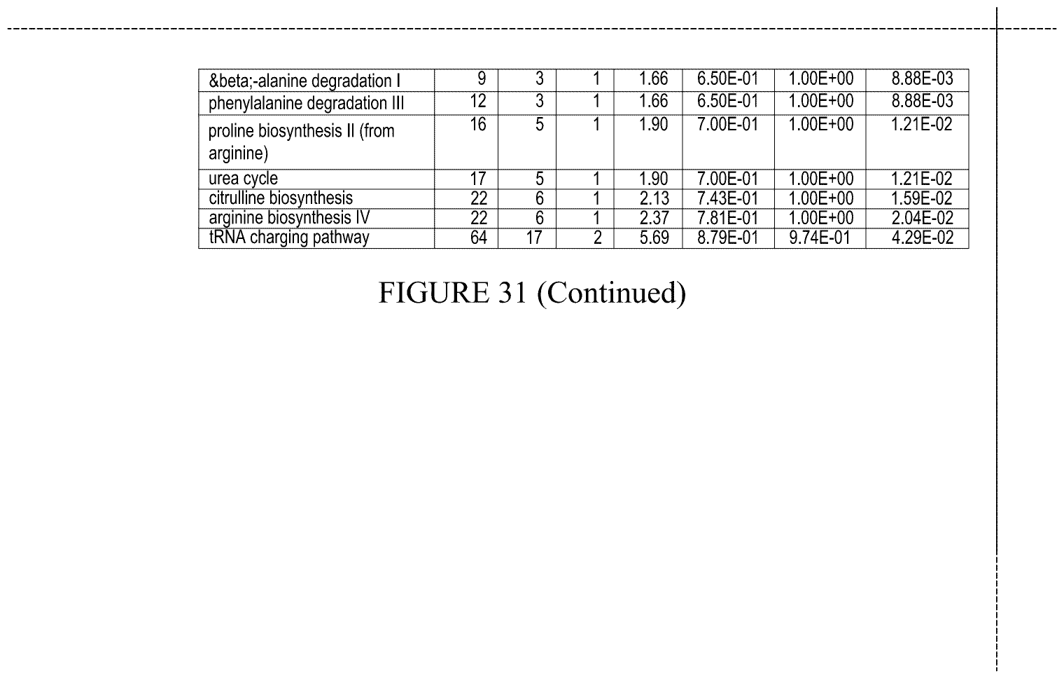

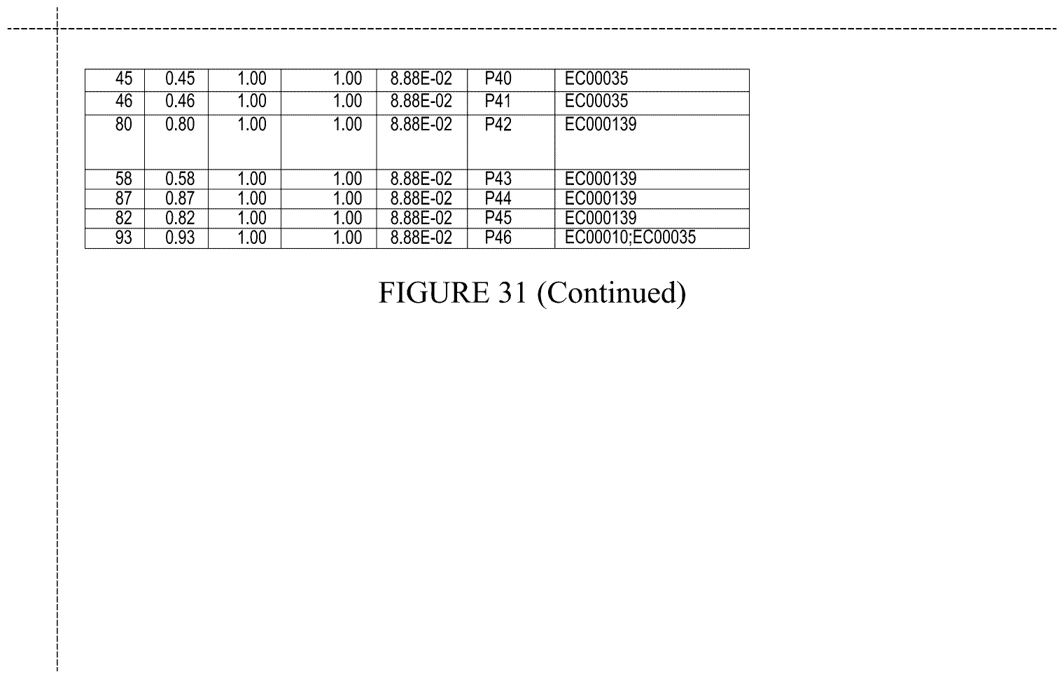

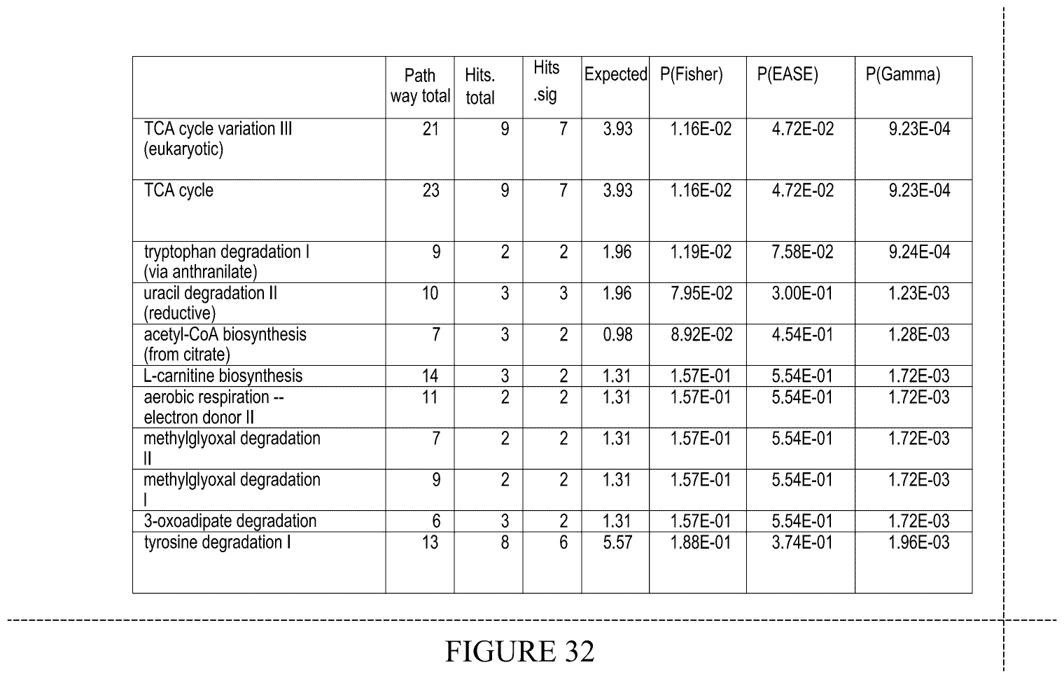

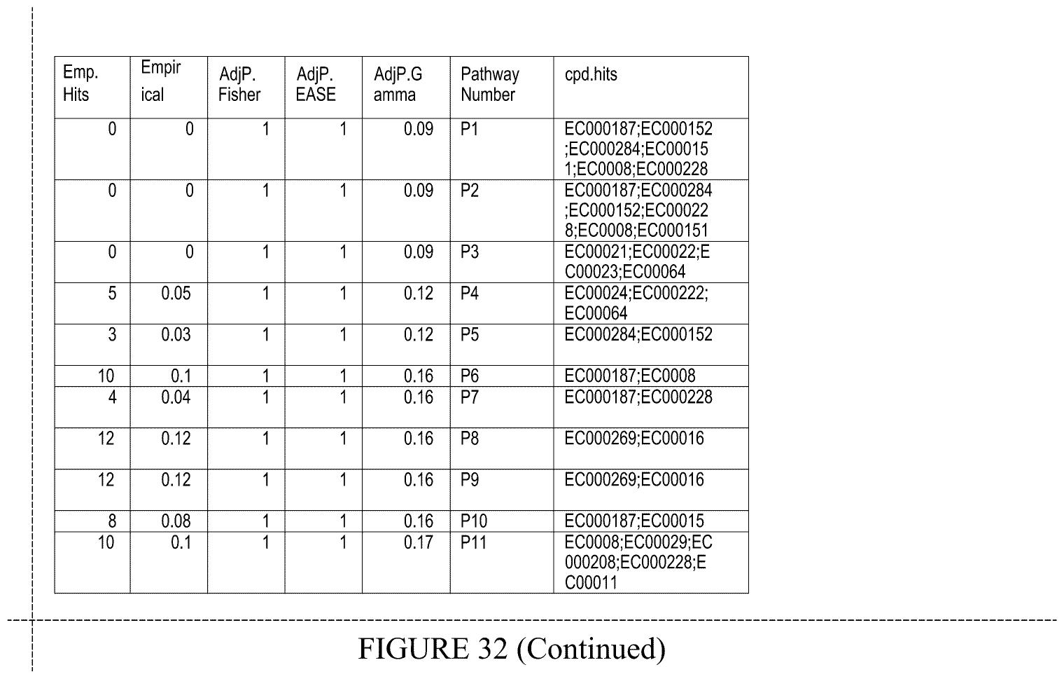

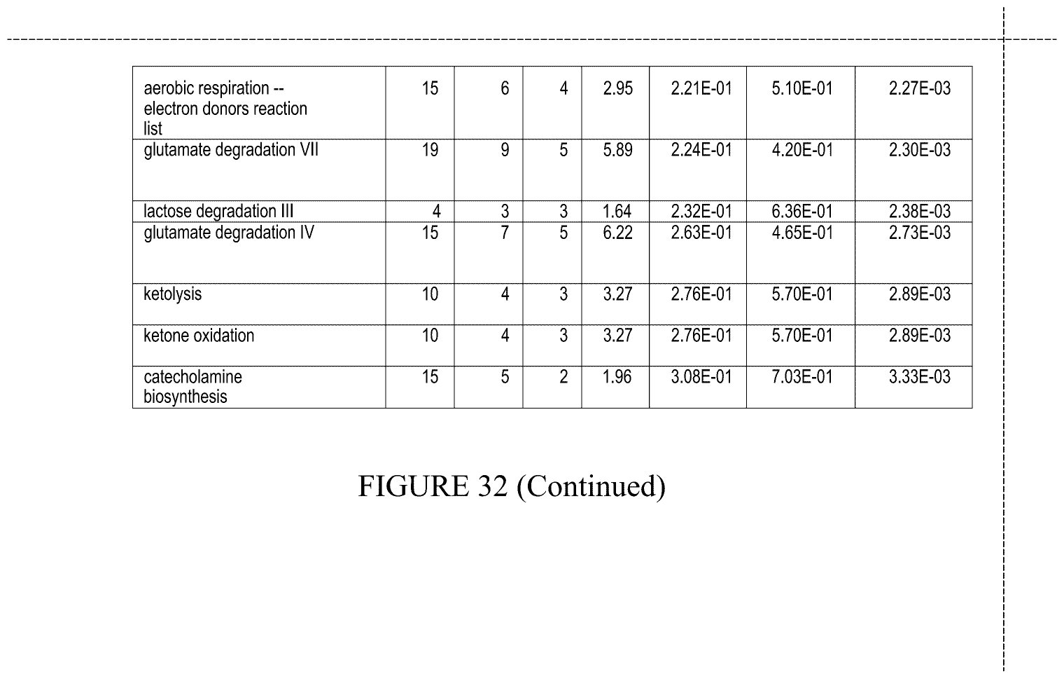

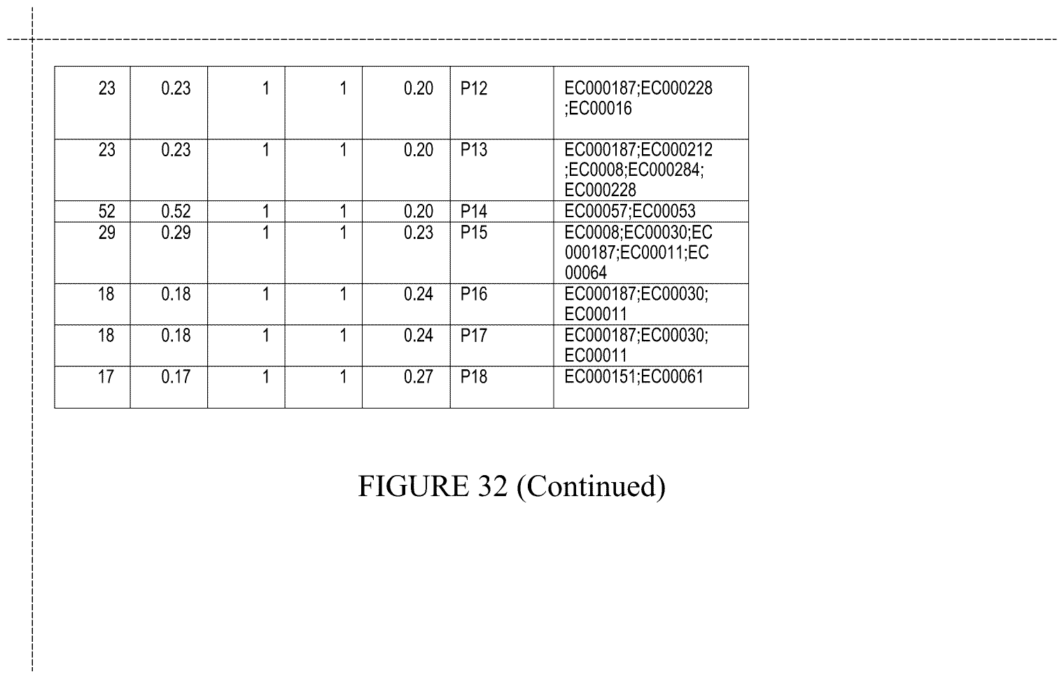

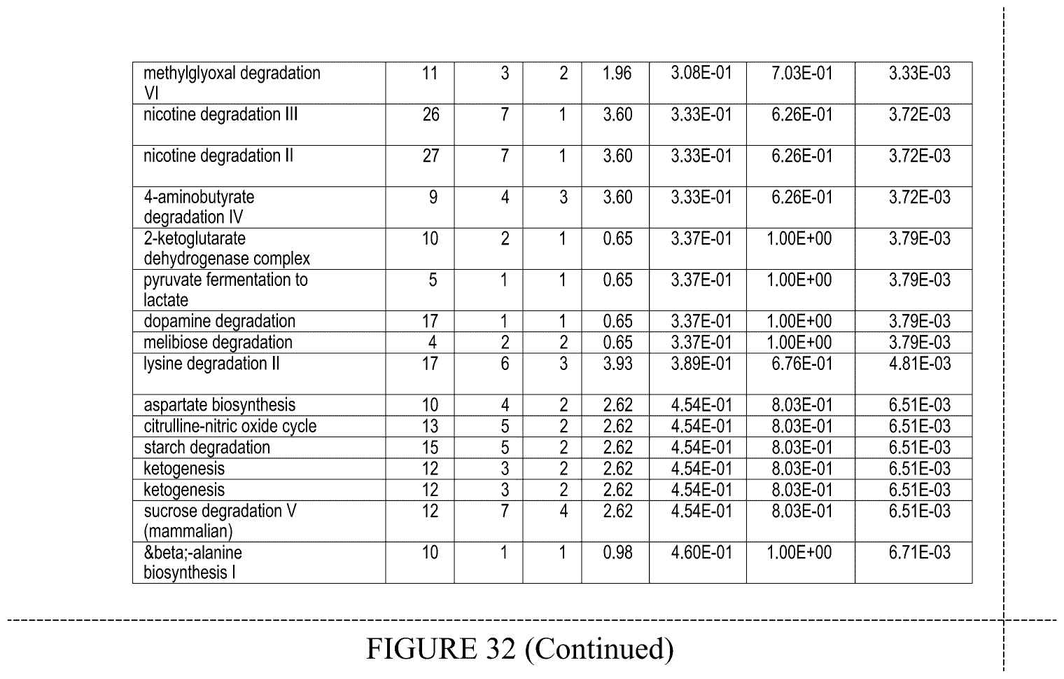

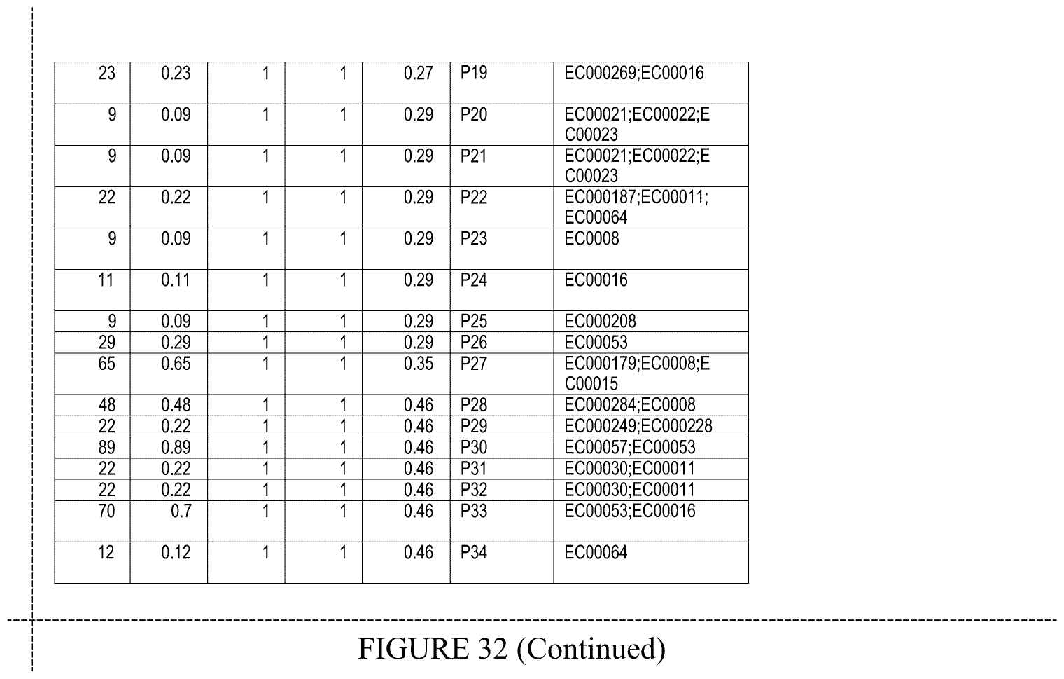

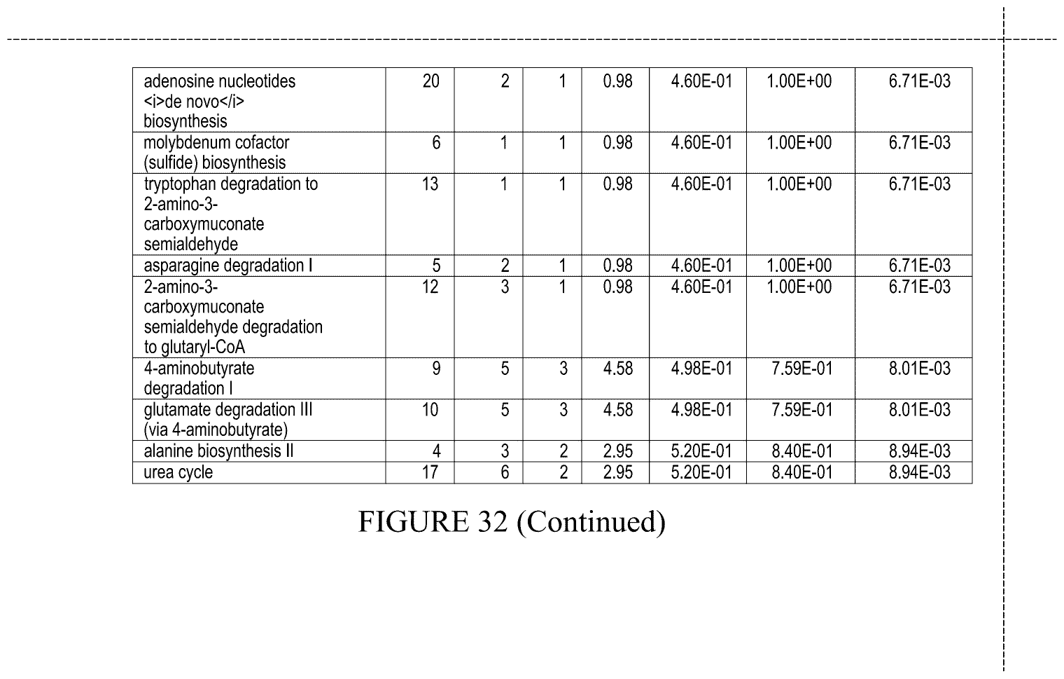

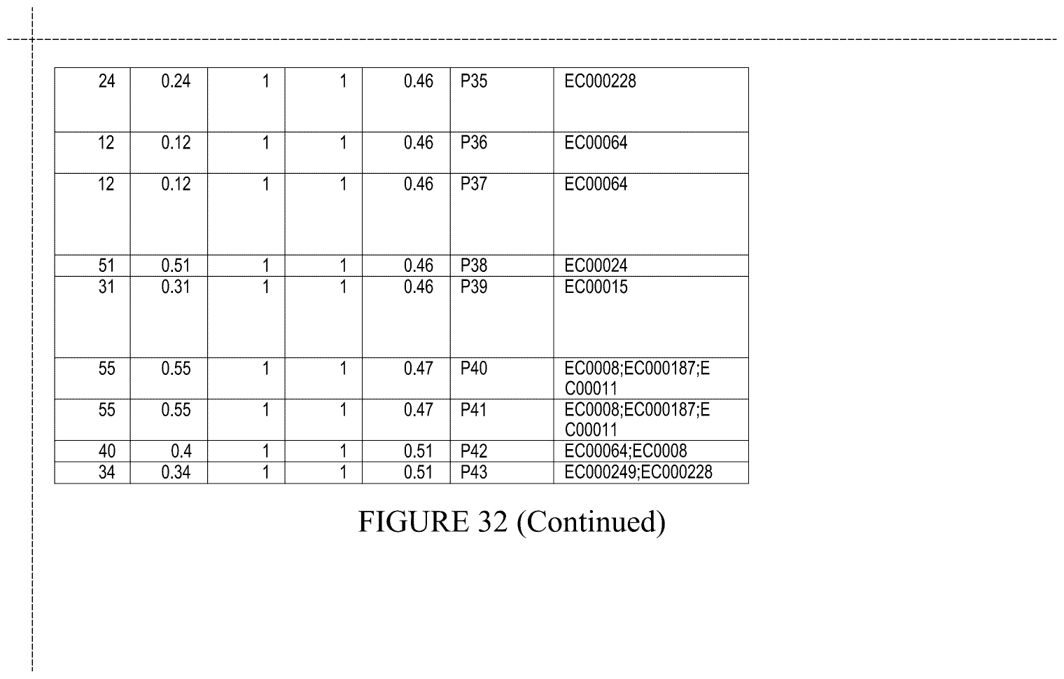

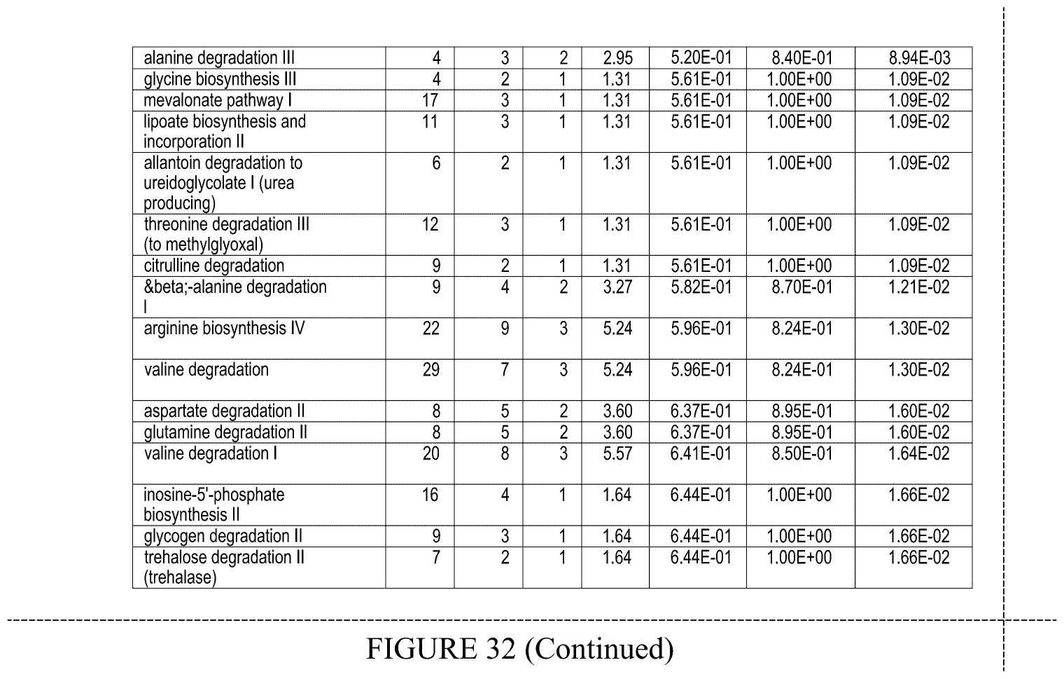

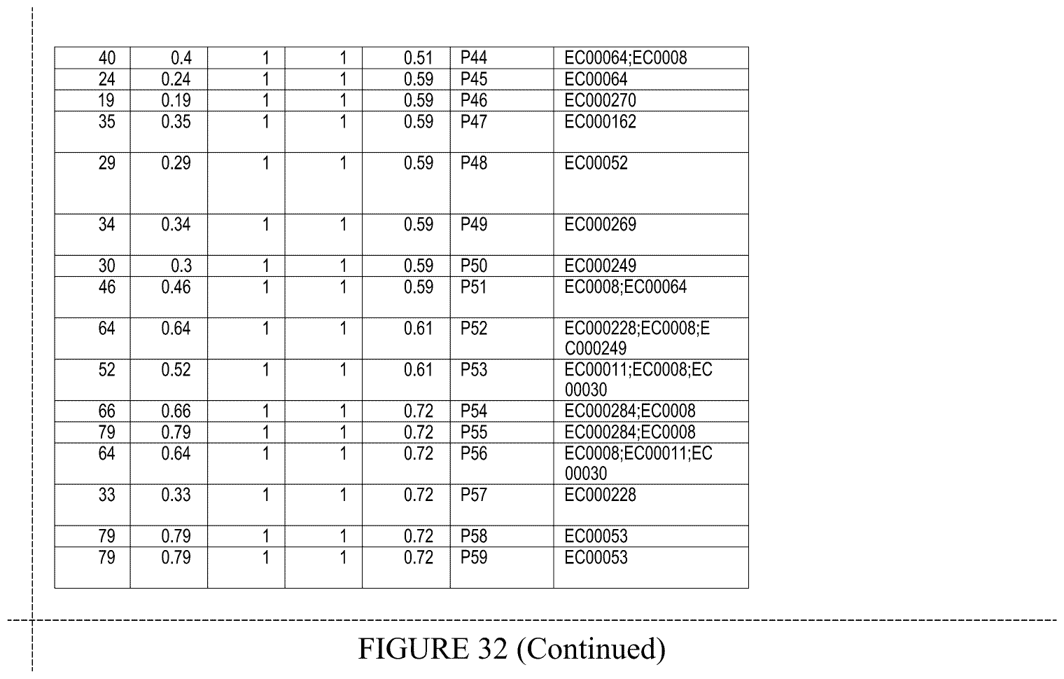

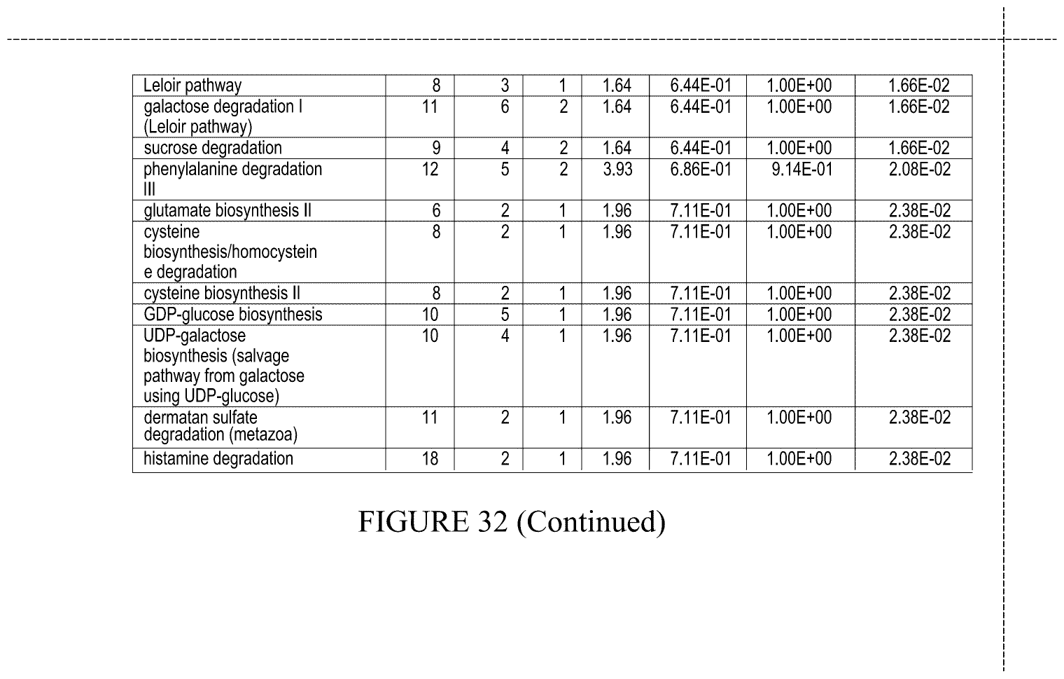

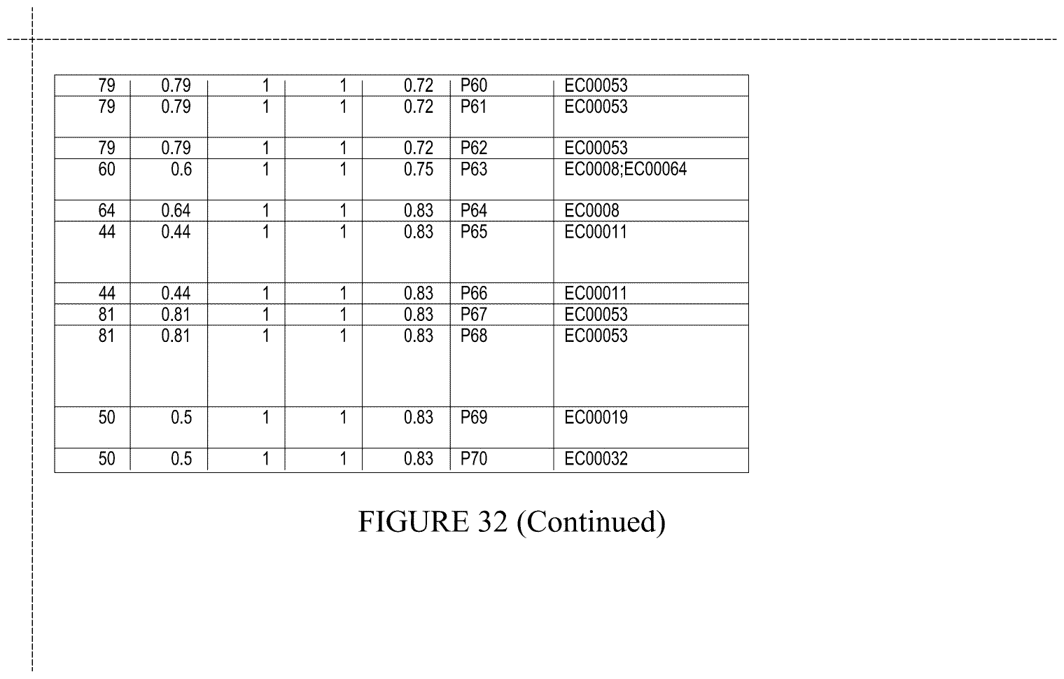

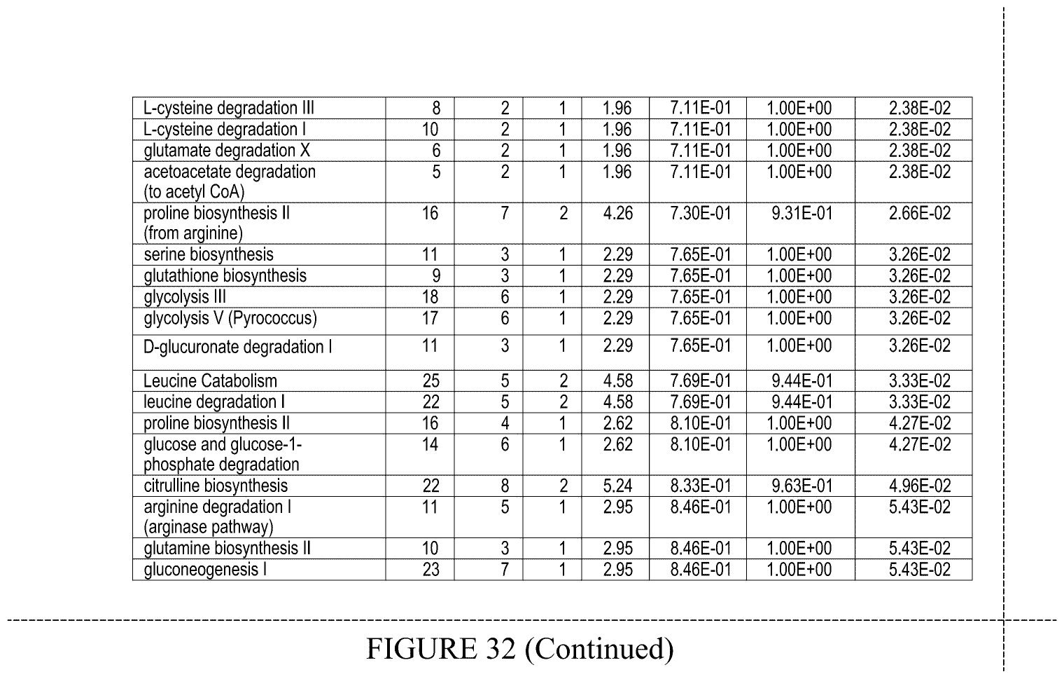

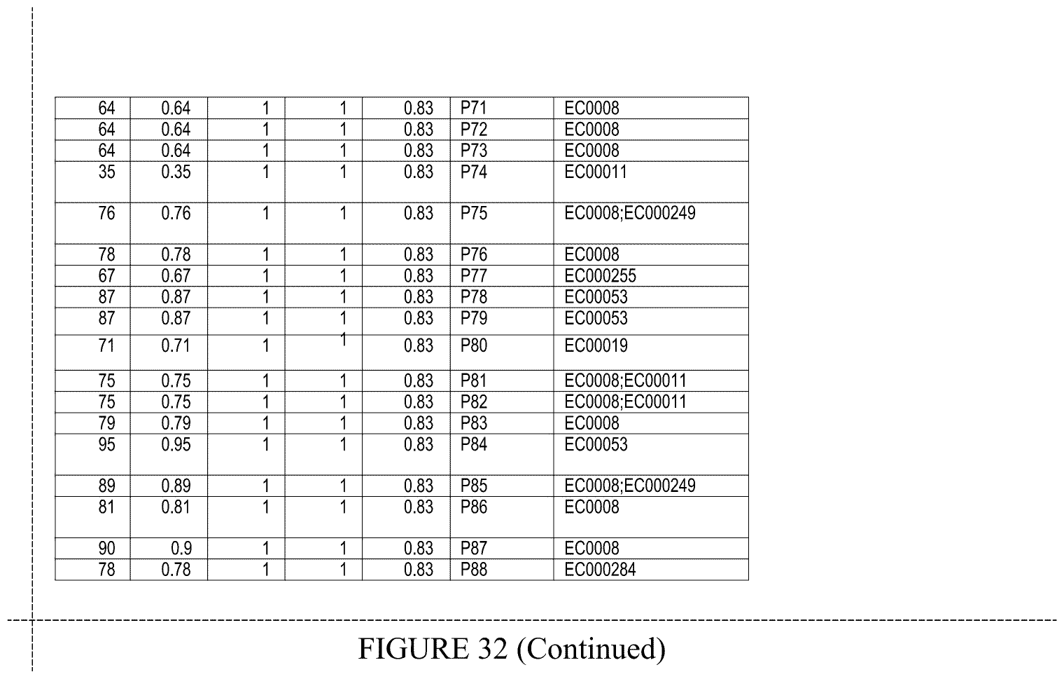

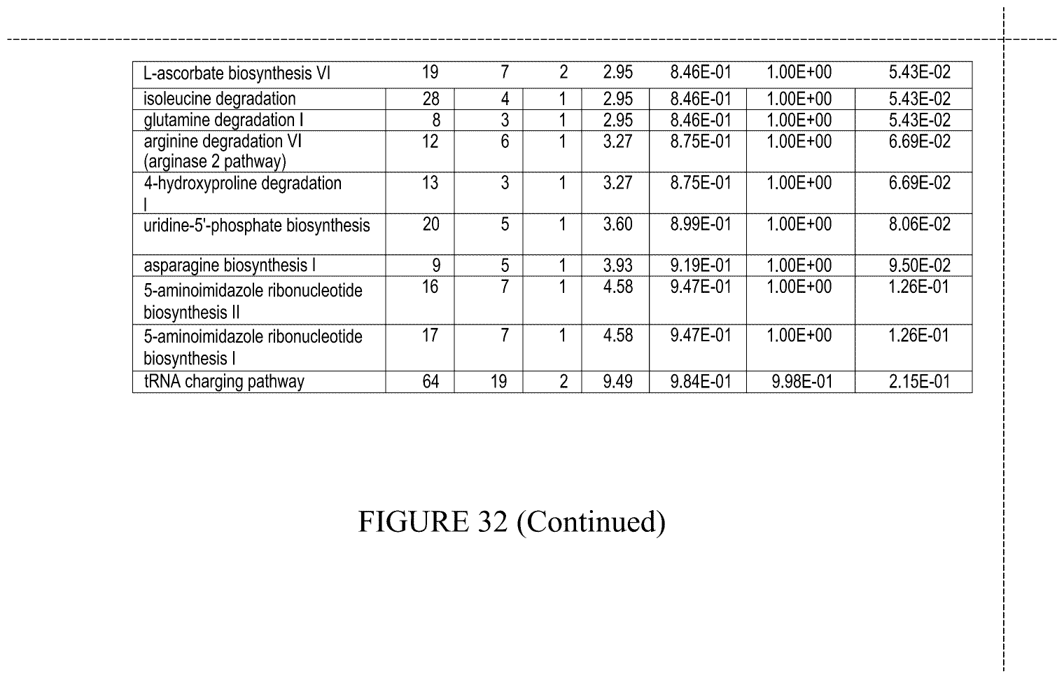

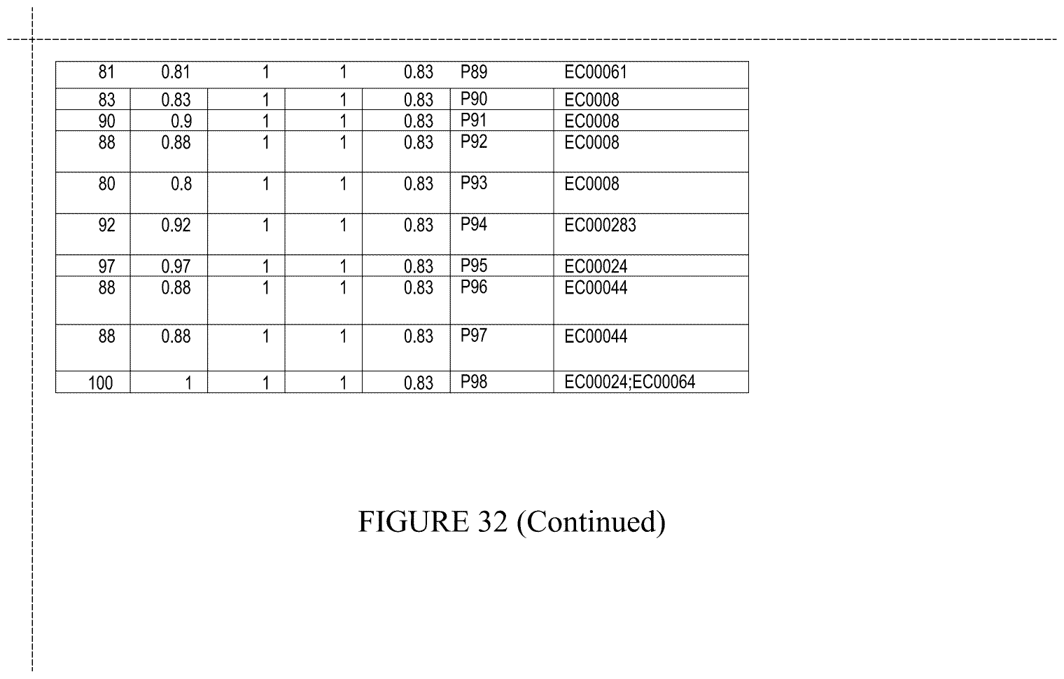

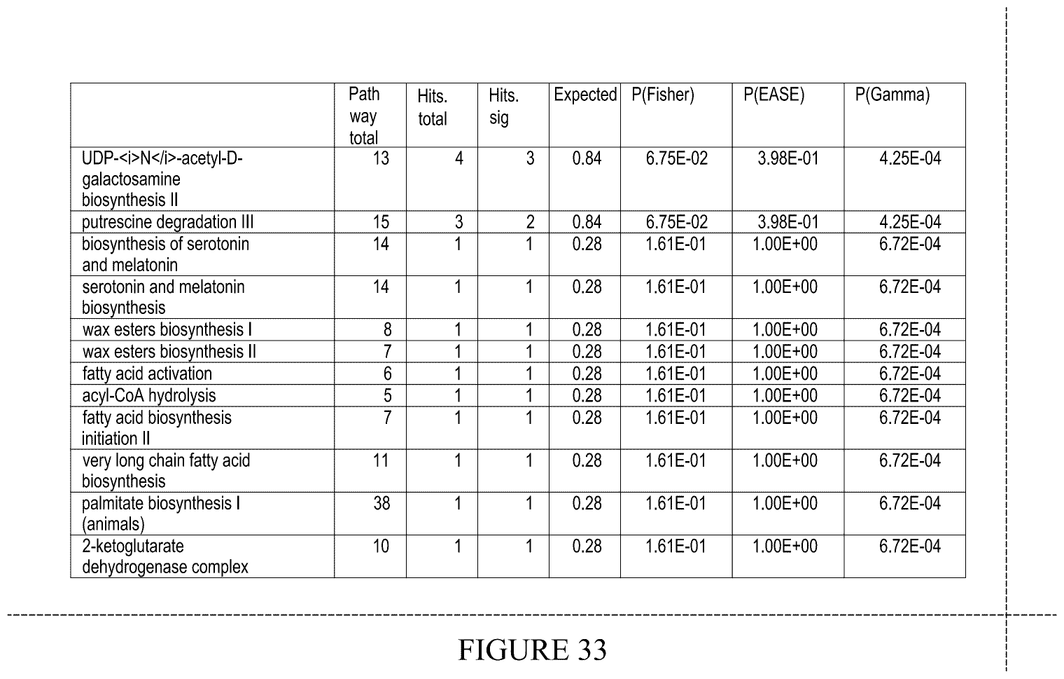

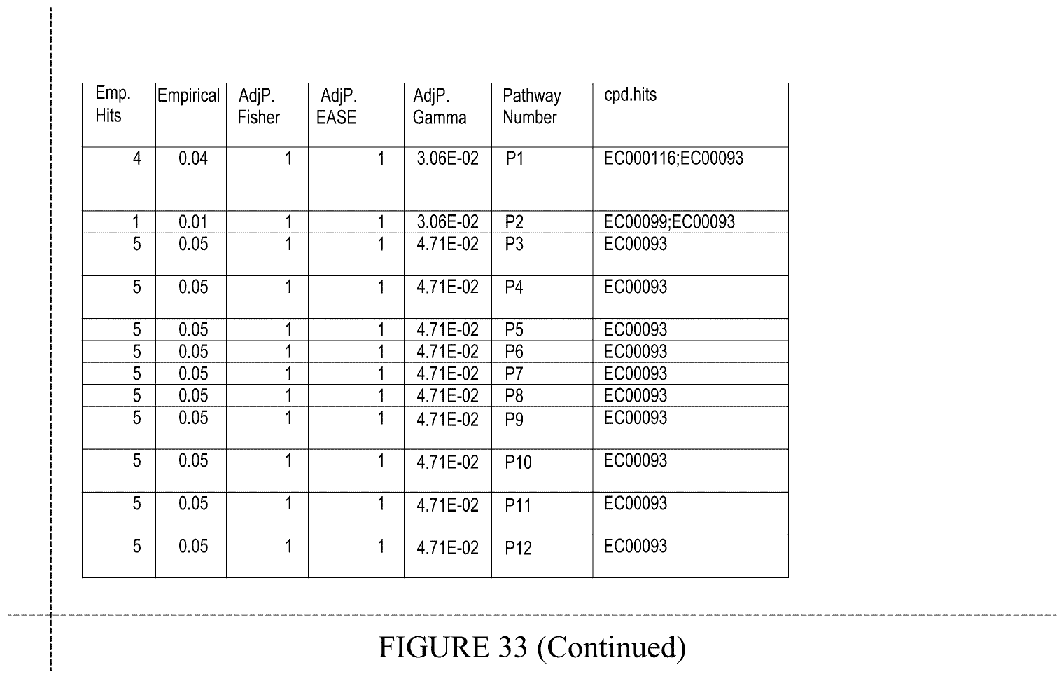

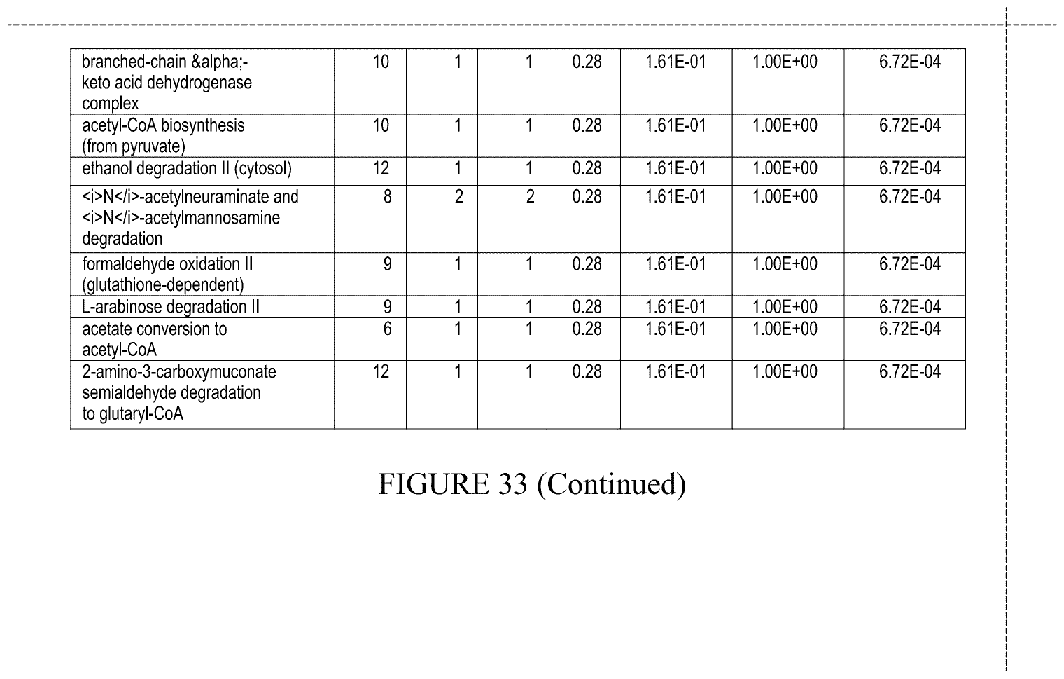

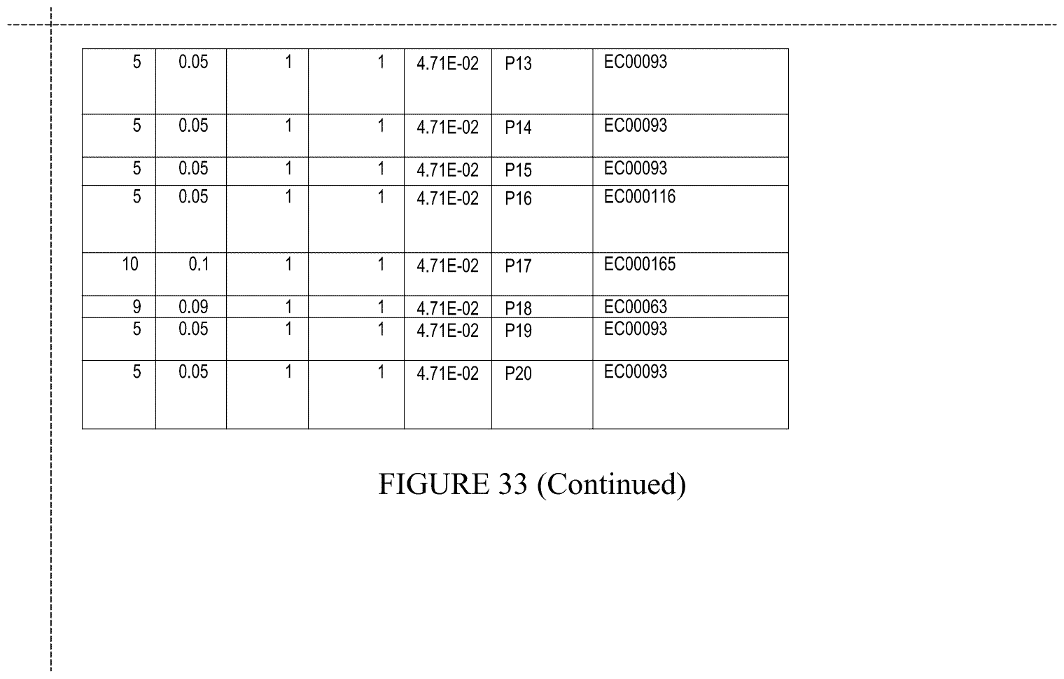

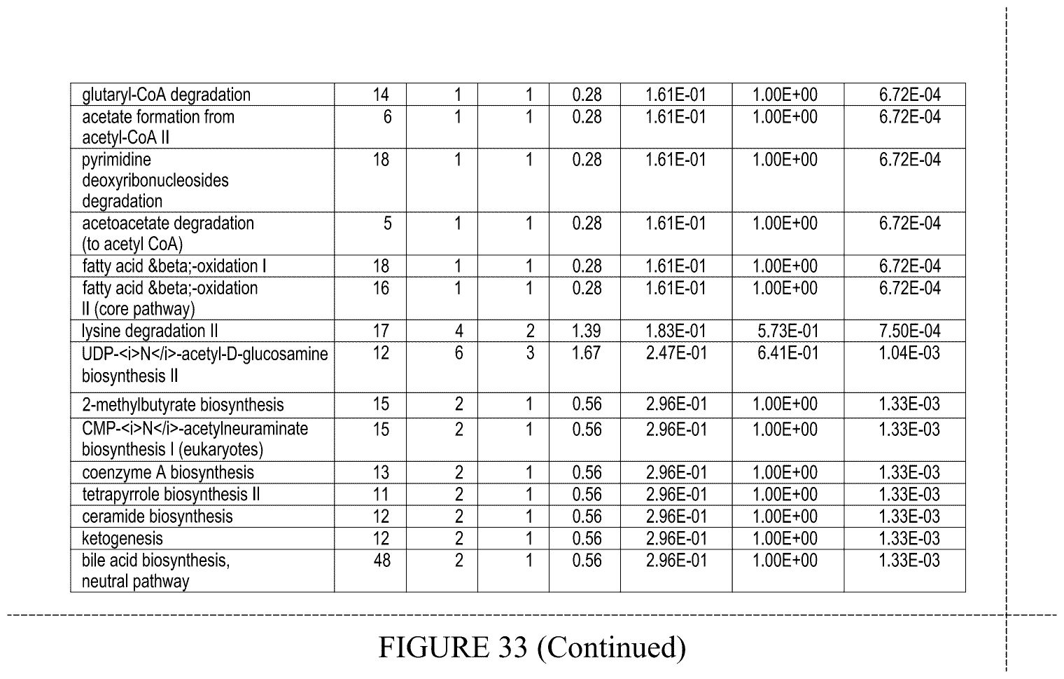

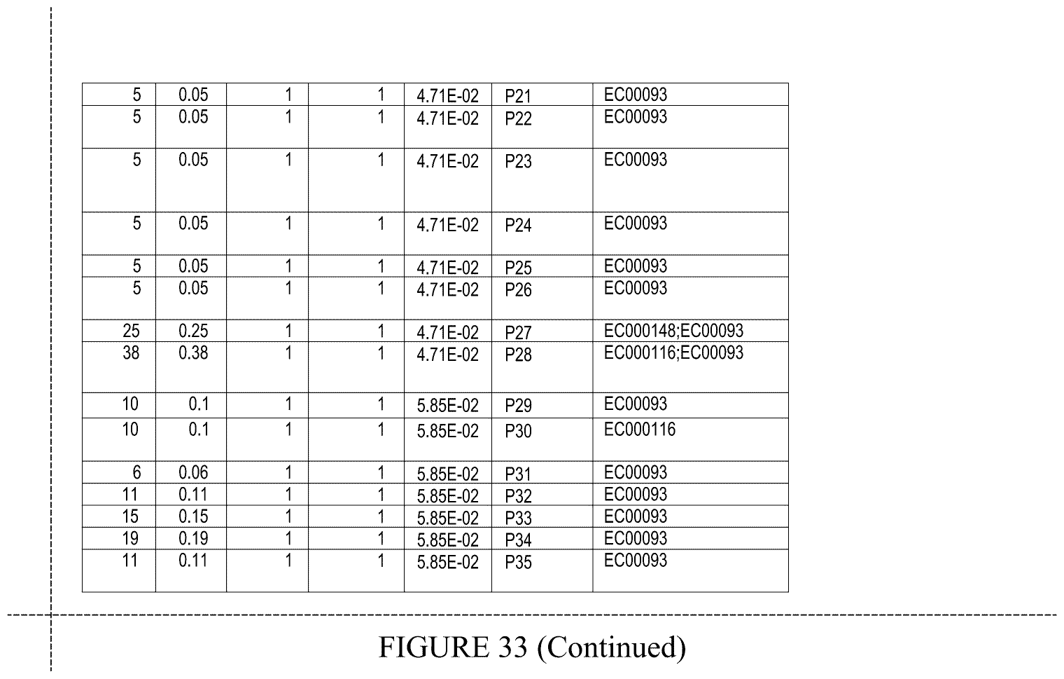

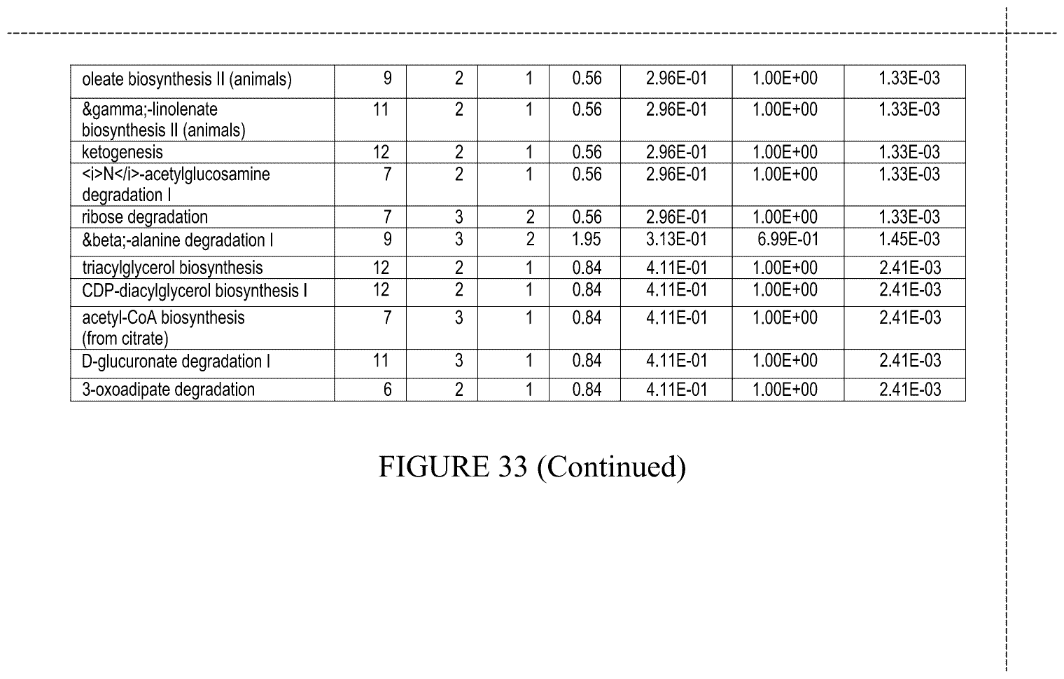

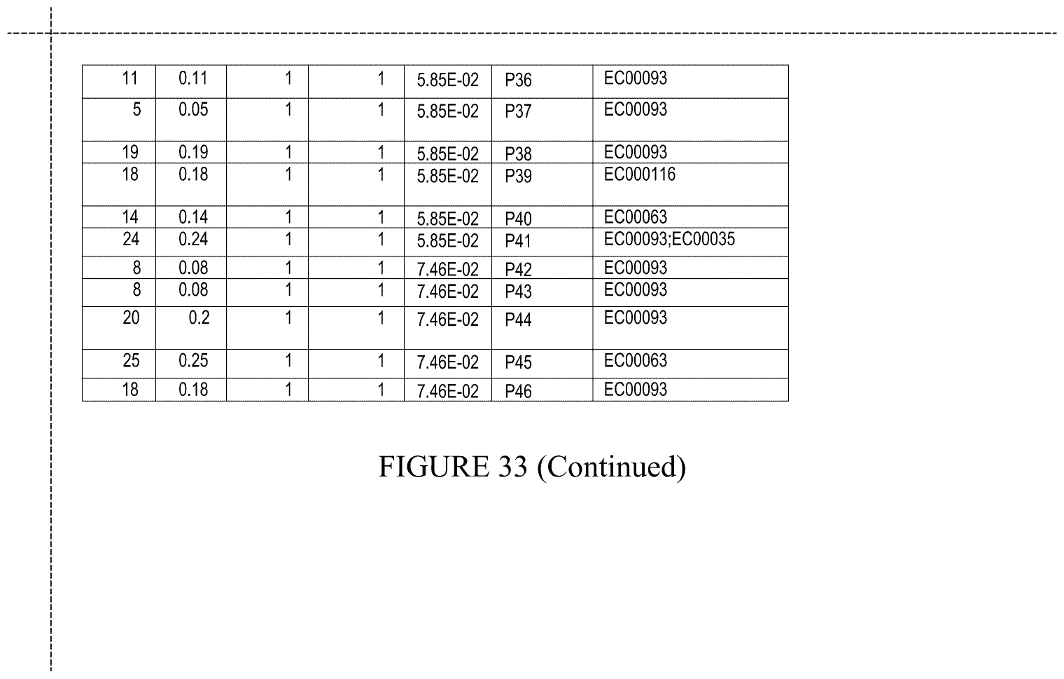

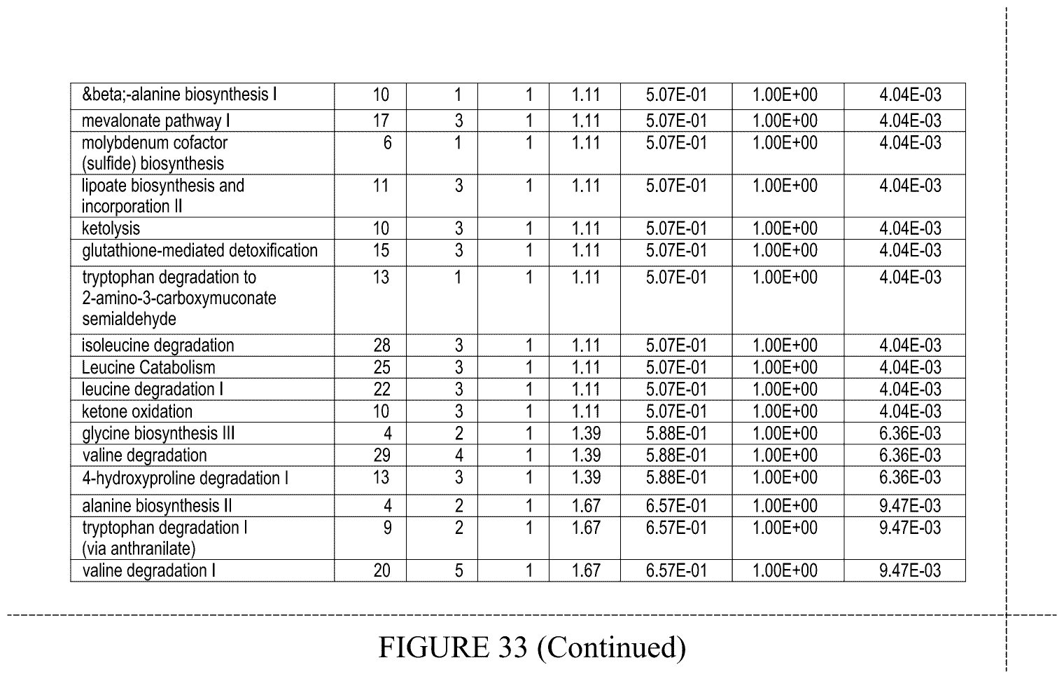

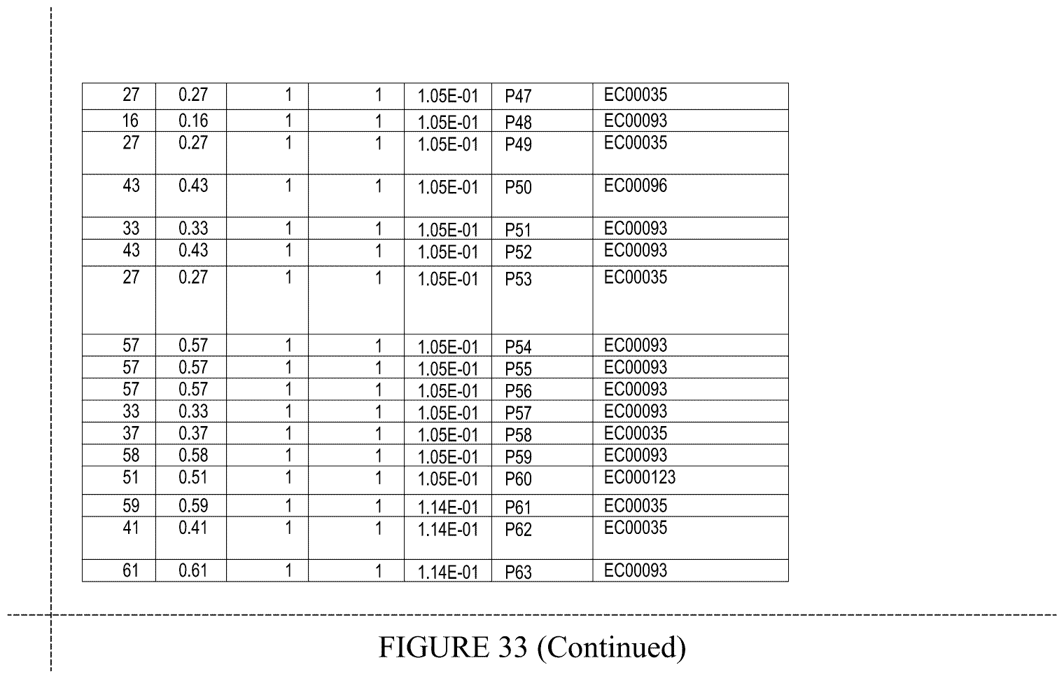

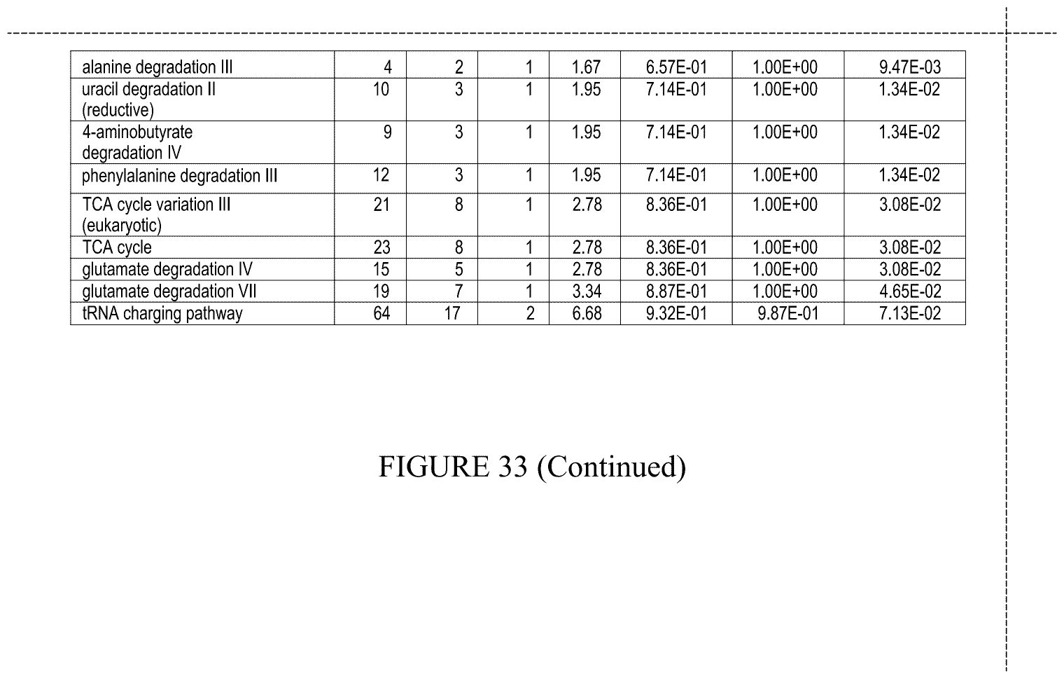

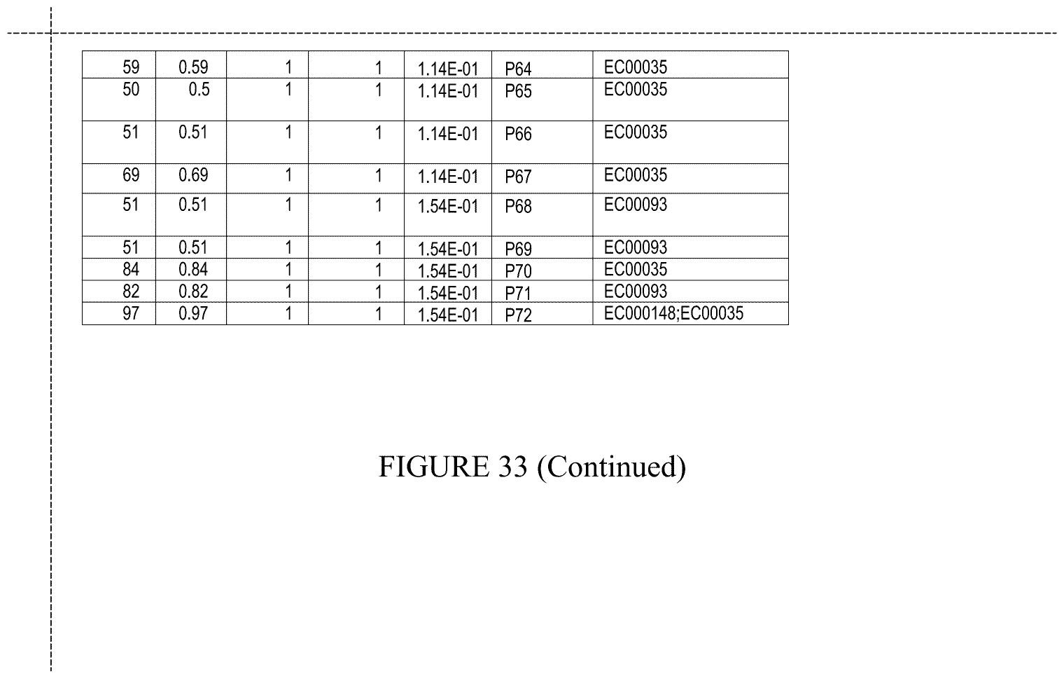

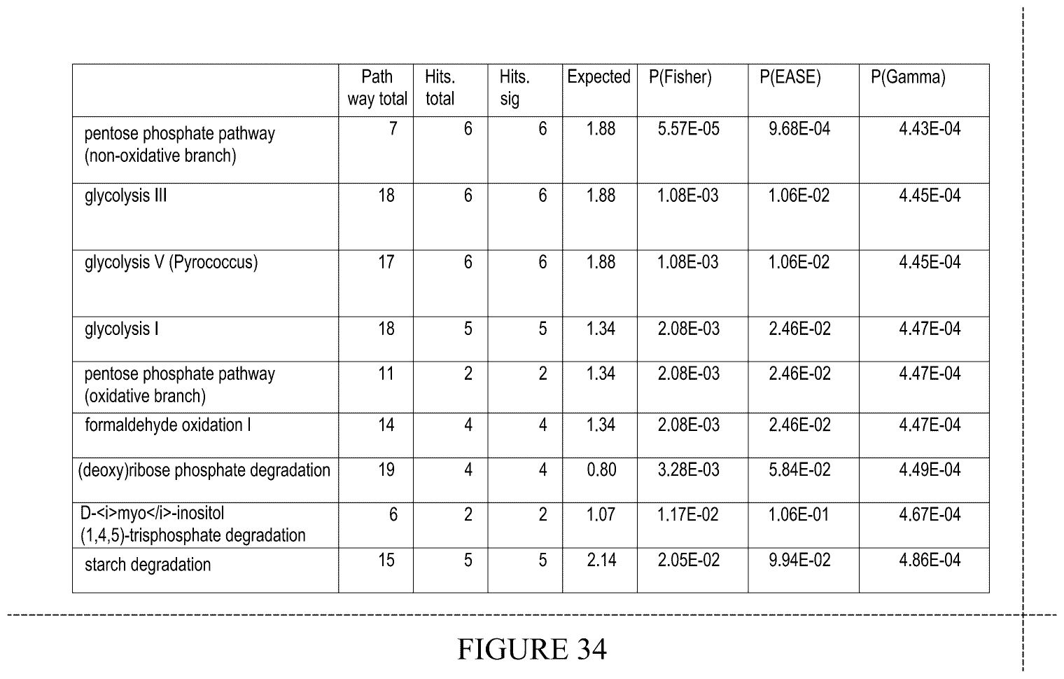

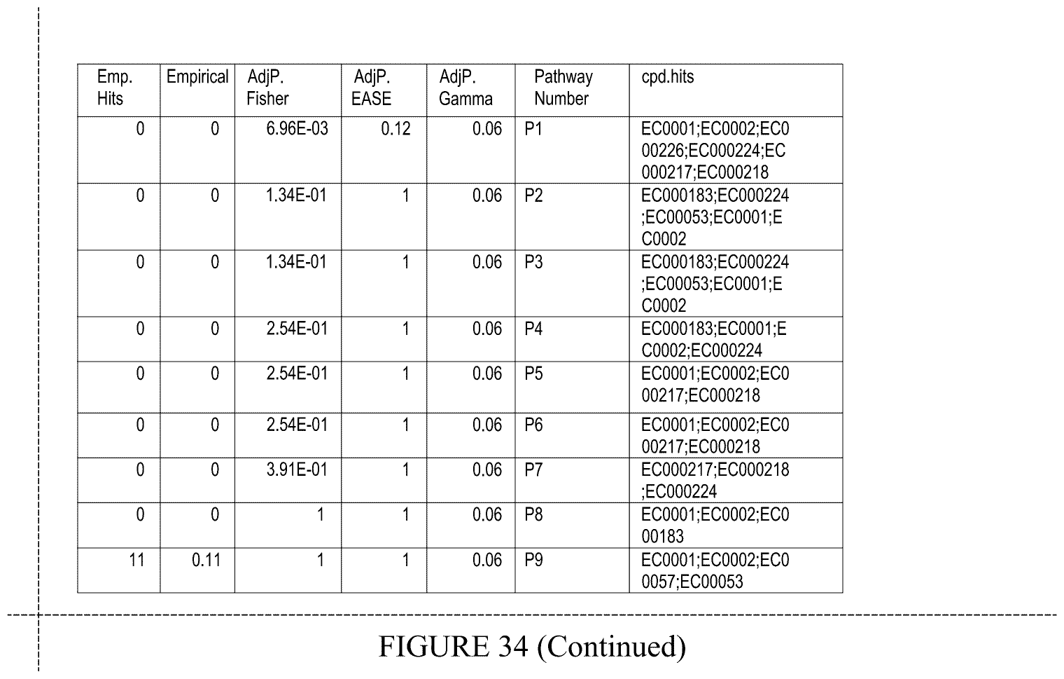

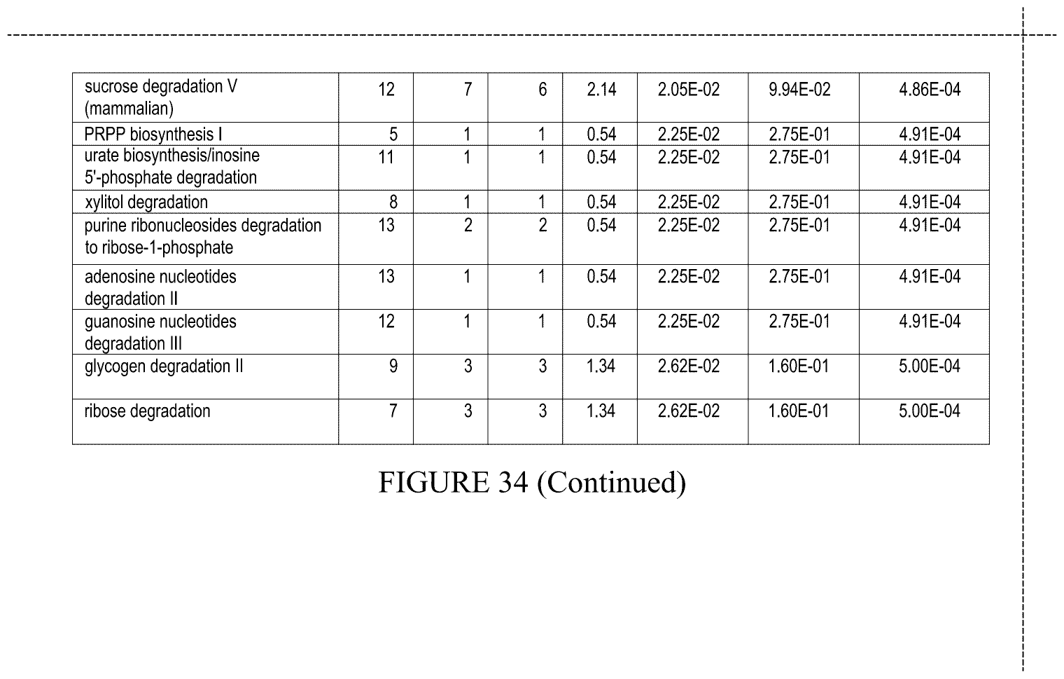

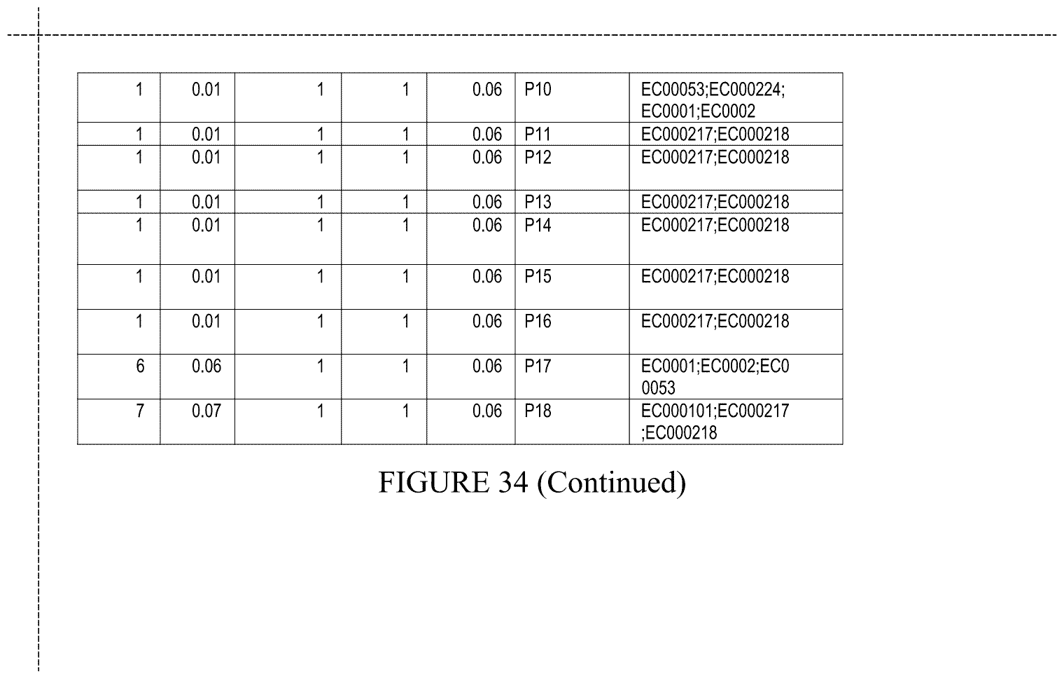

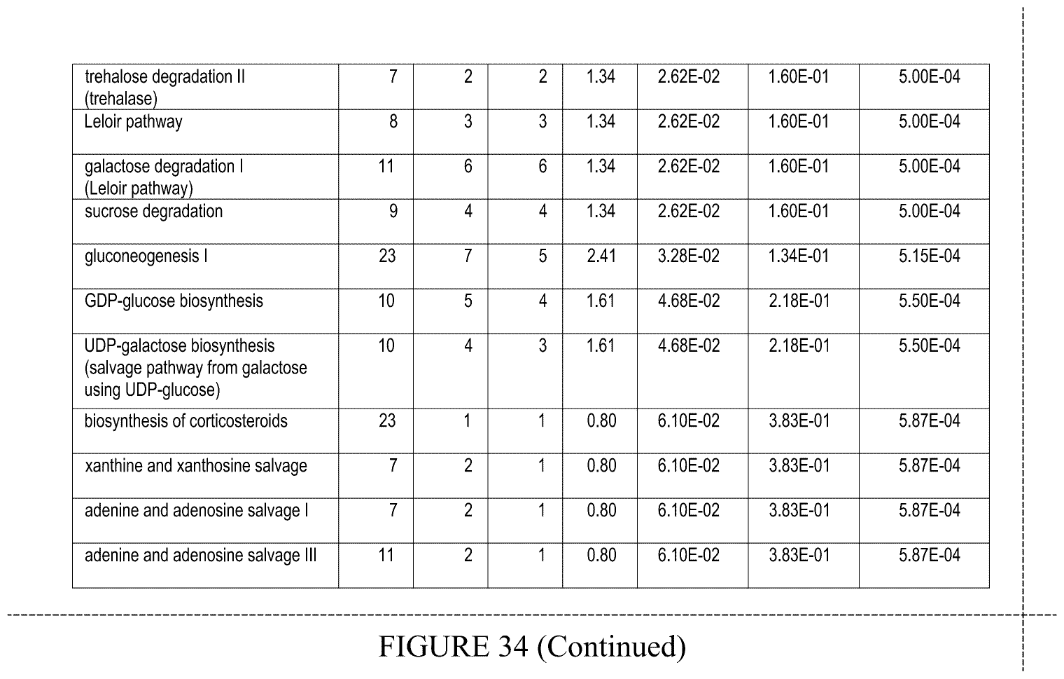

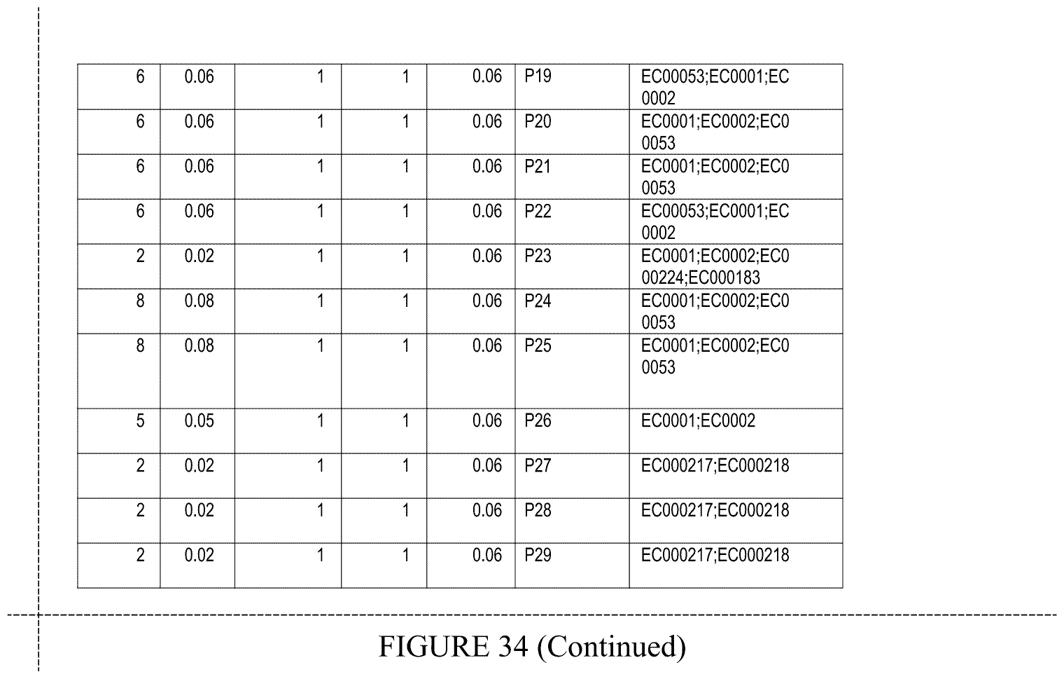

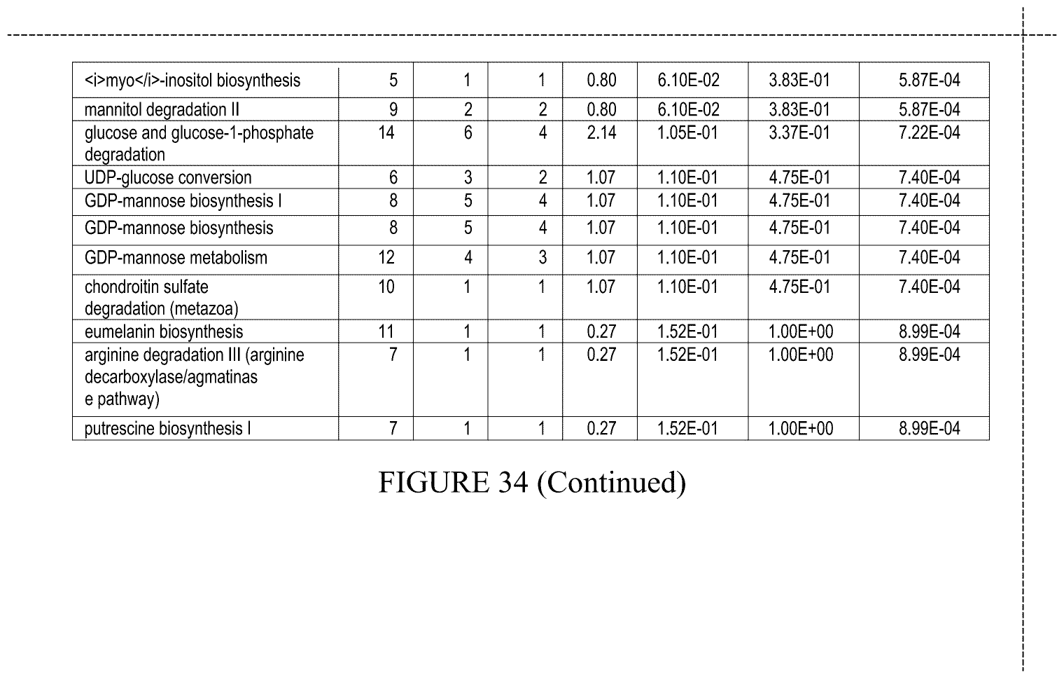

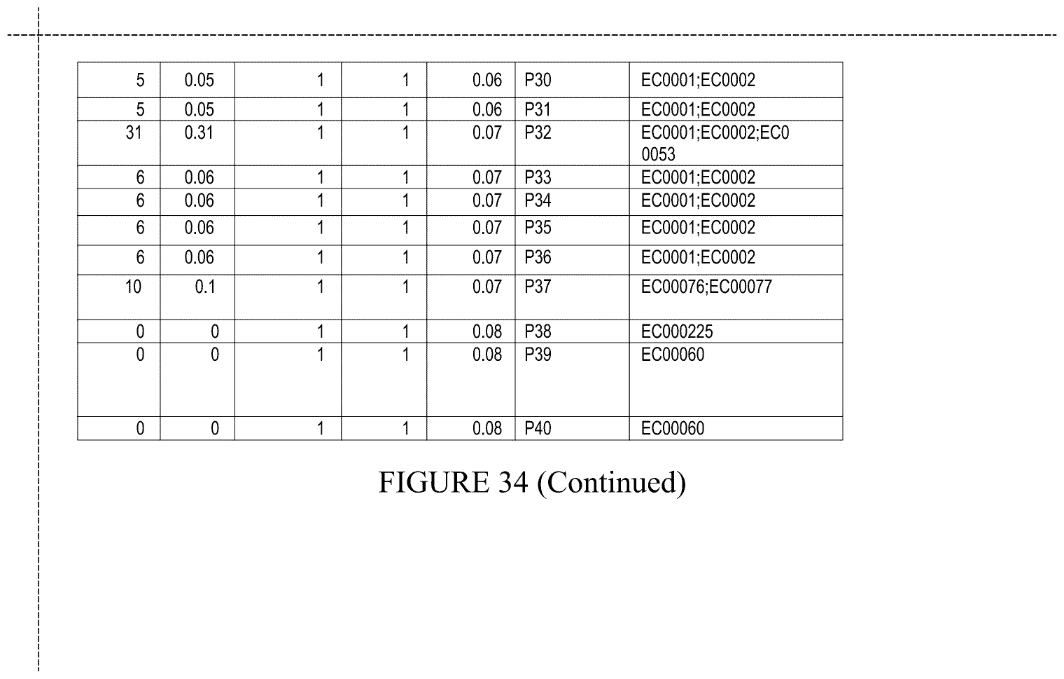

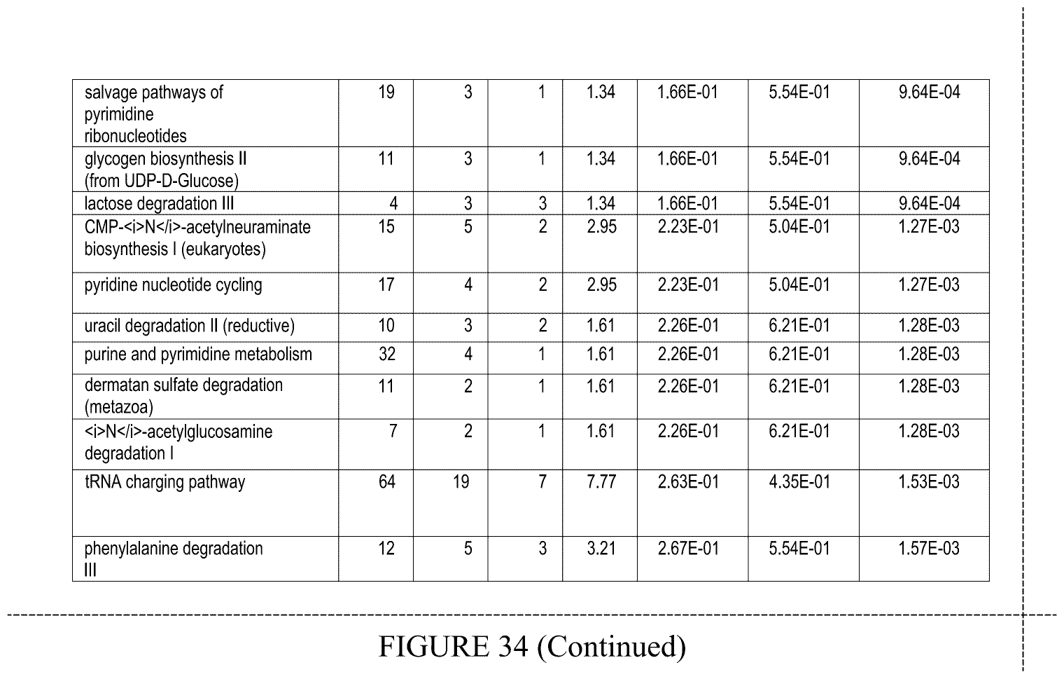

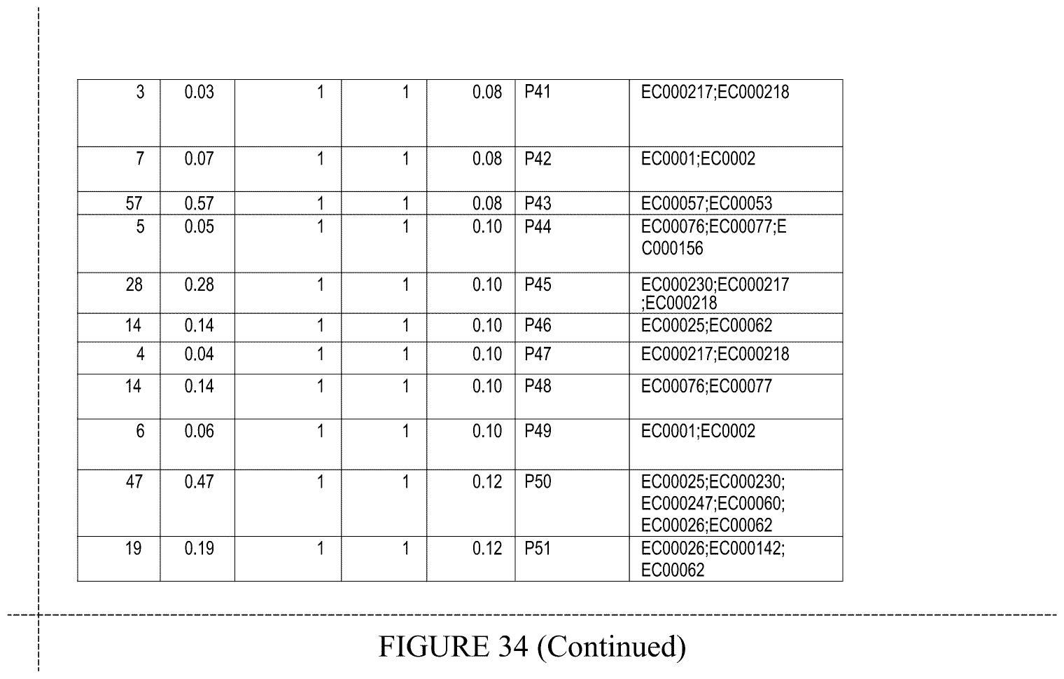

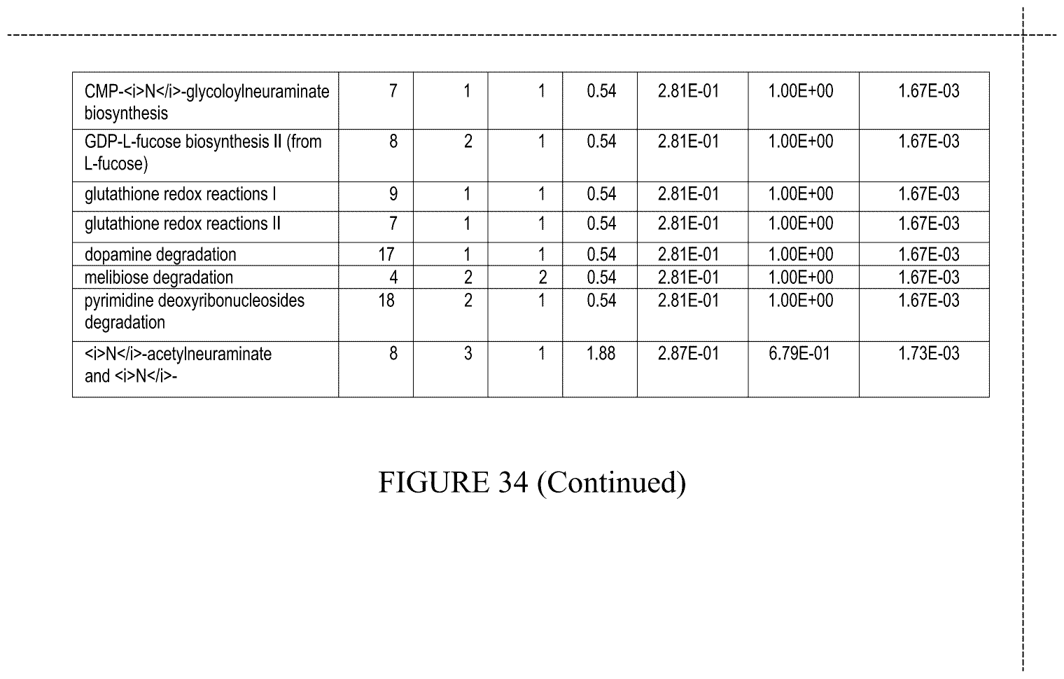

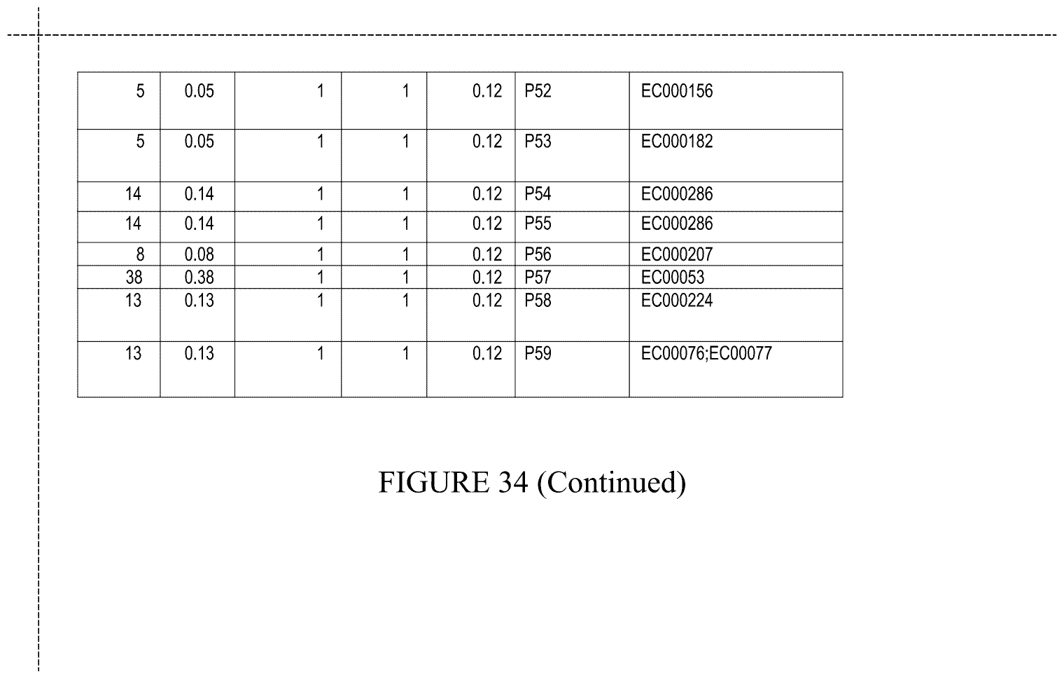

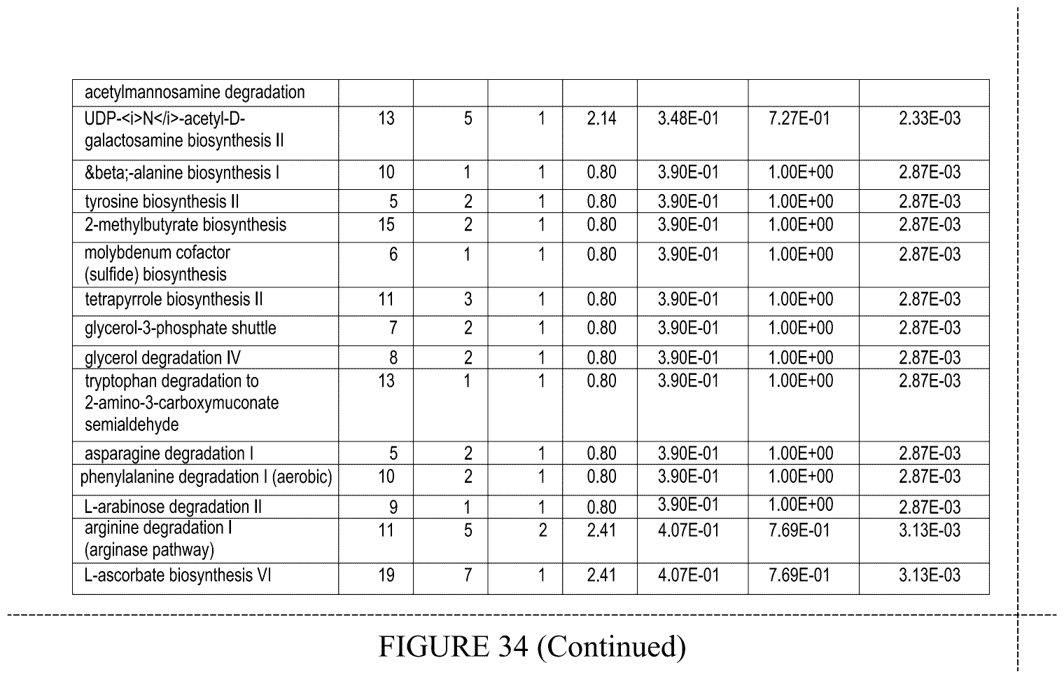

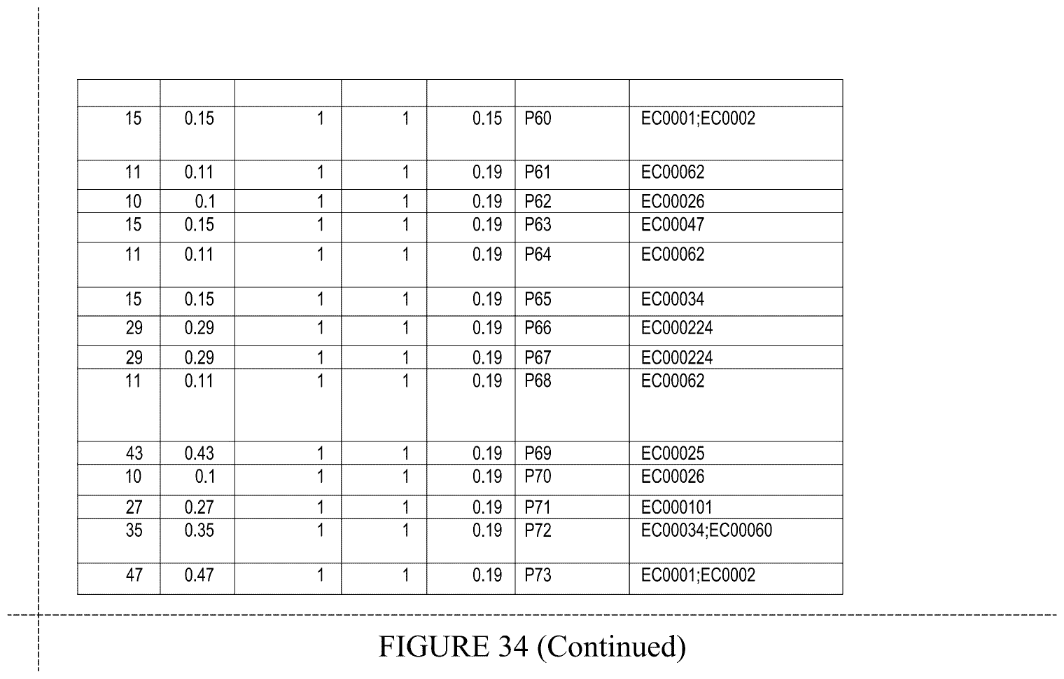

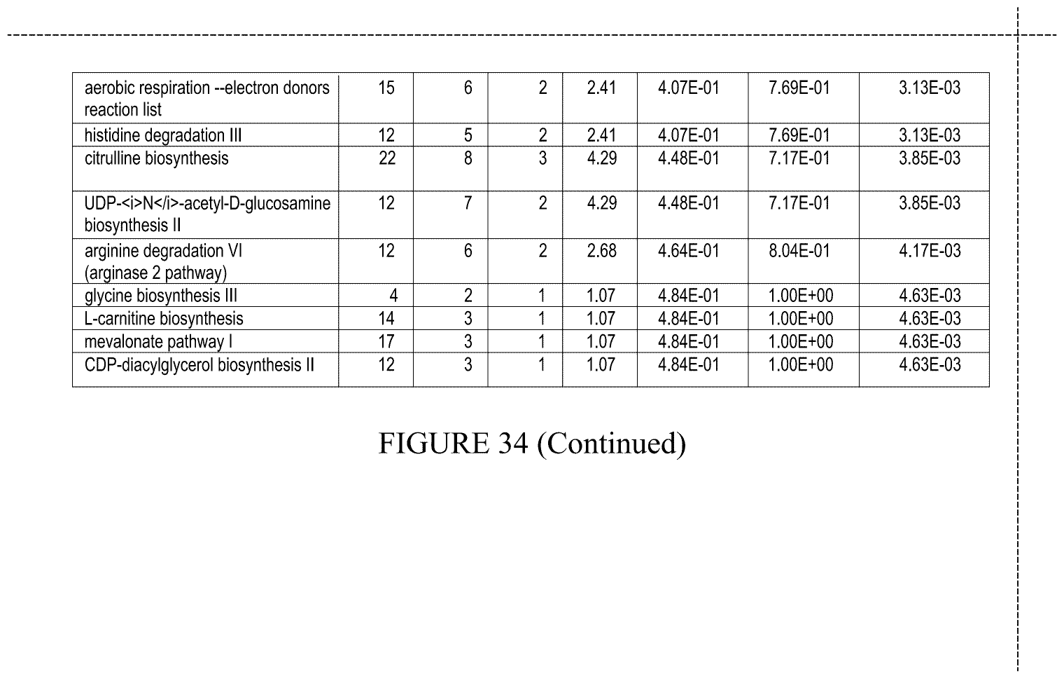

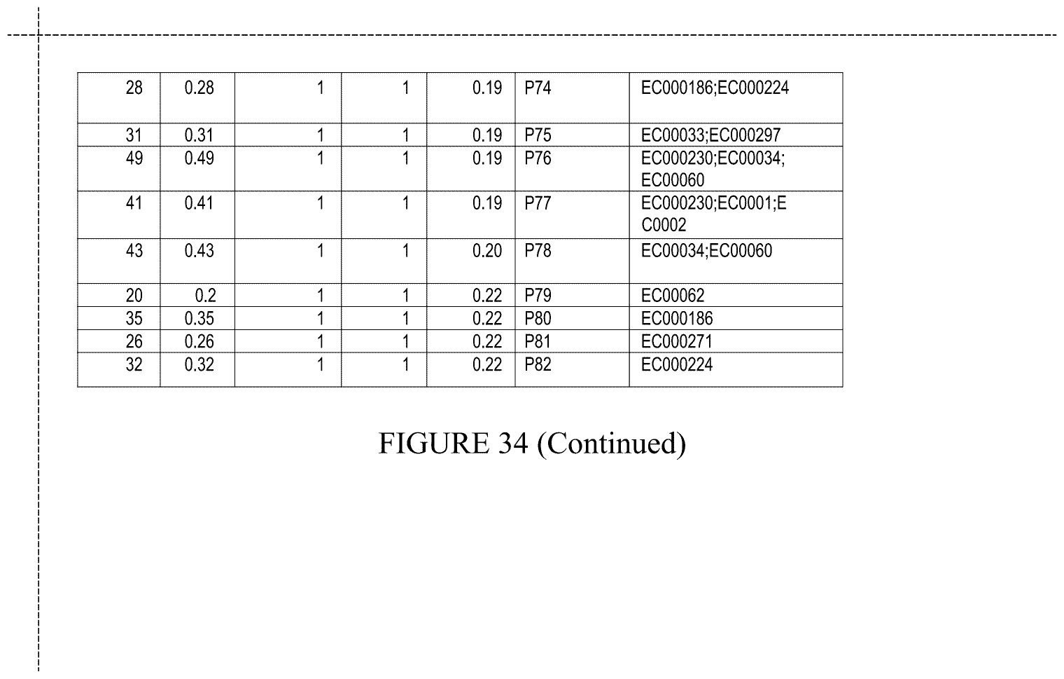

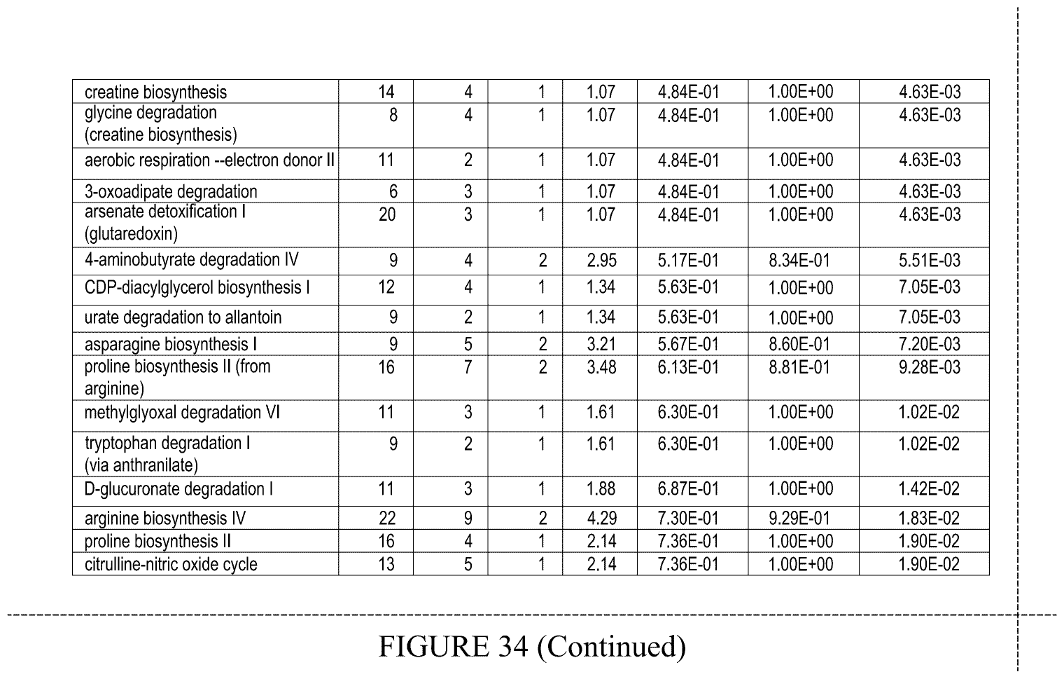

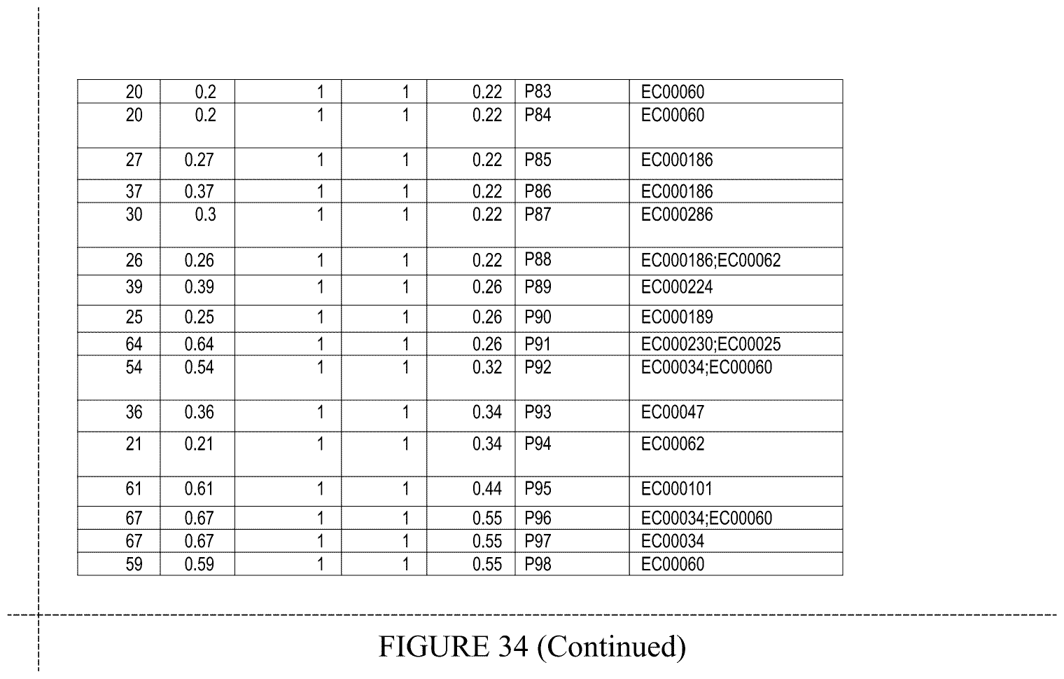

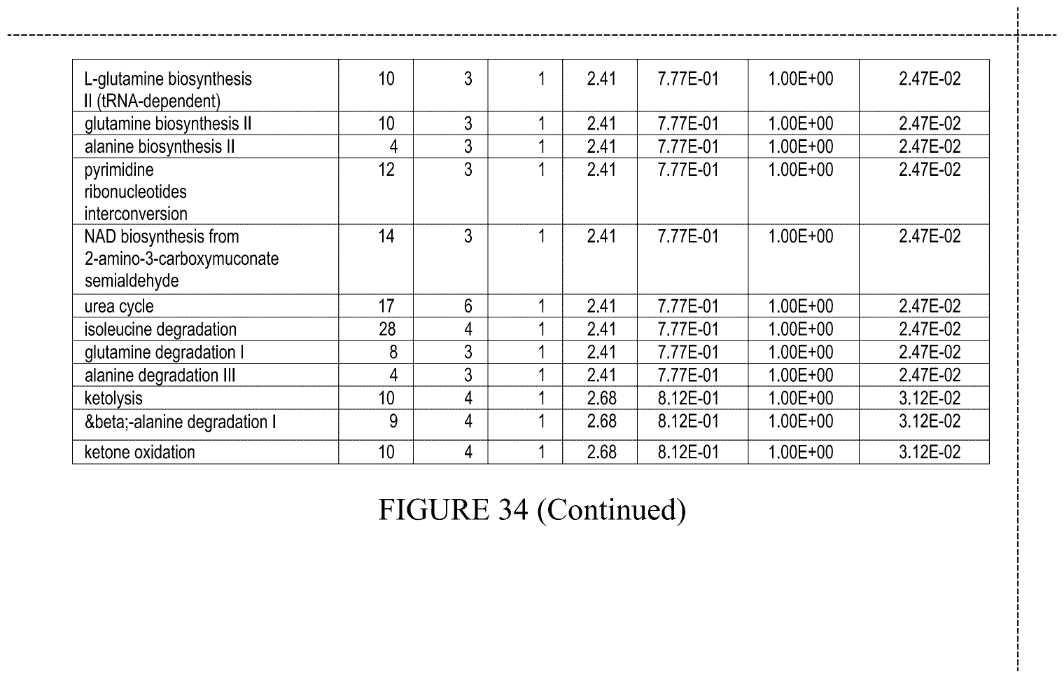

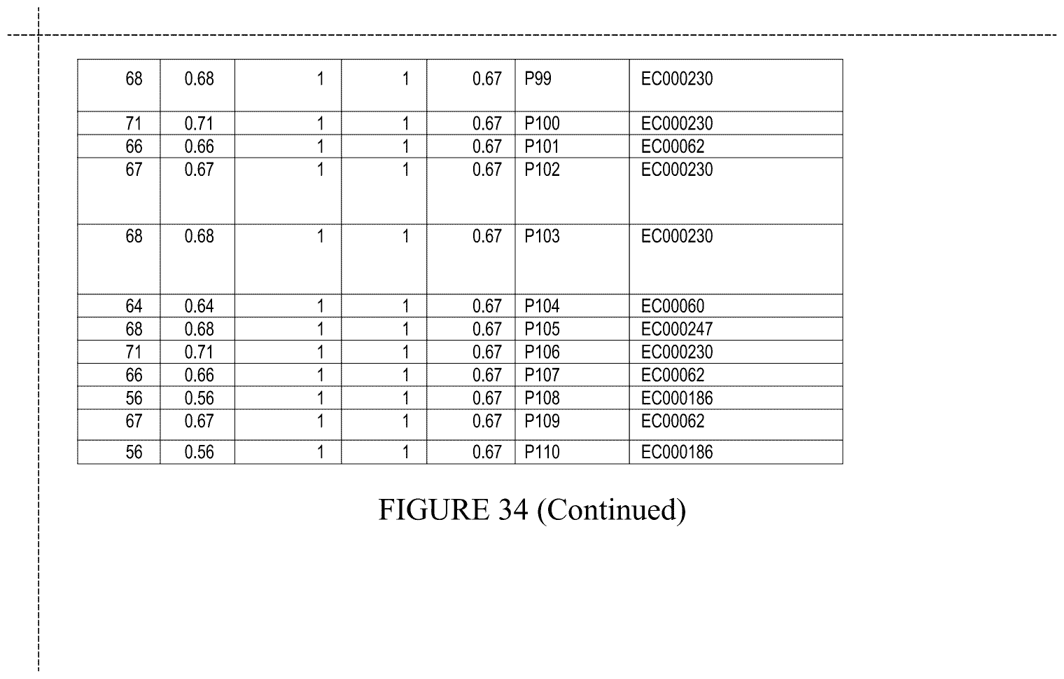

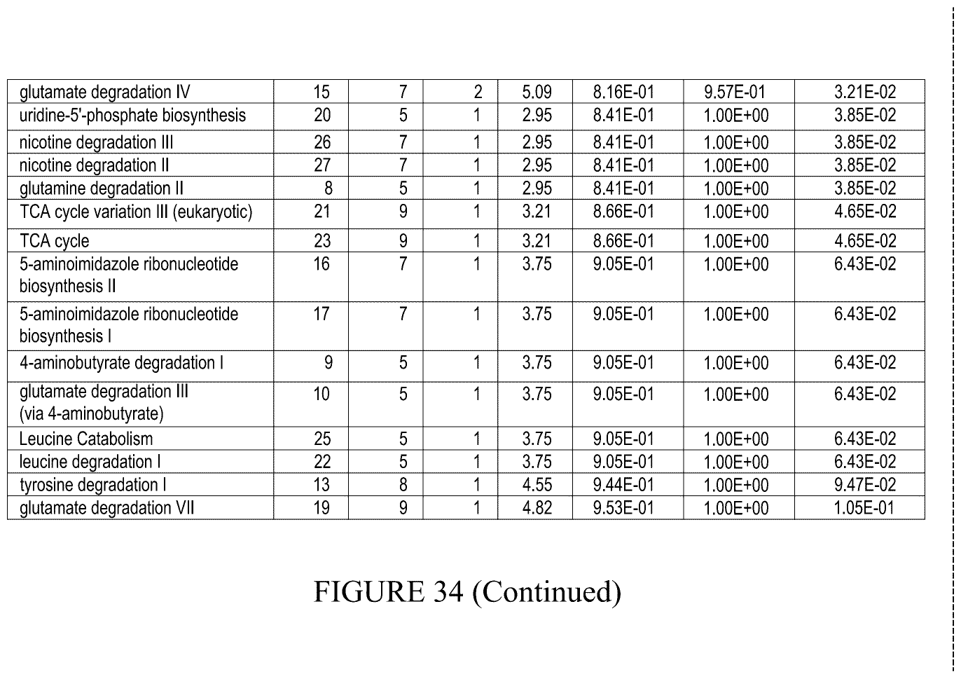

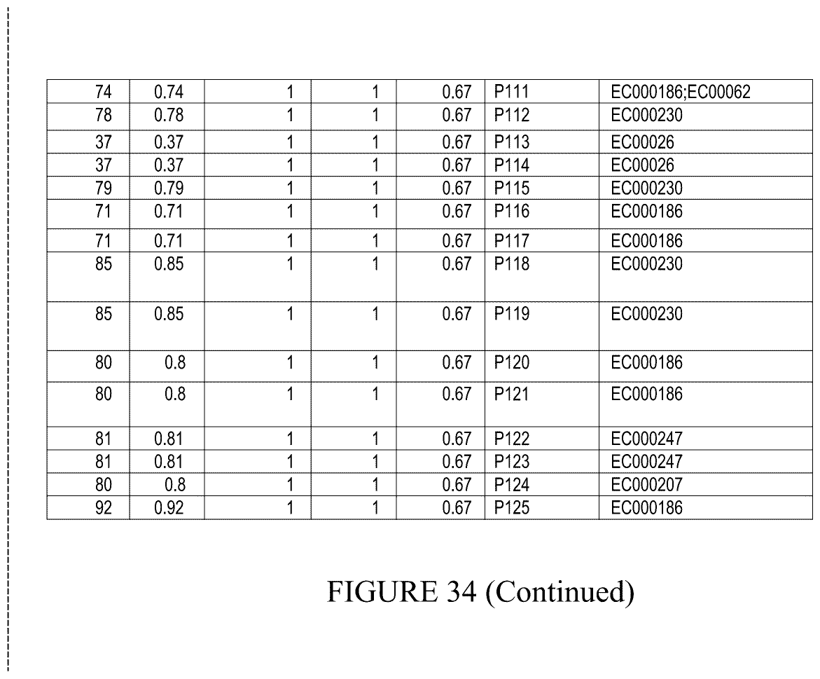

Adropin inhibits O-GlcNAcylation of FAO enzymes to restore substrate flexibility in HFpEF. Finally, co-immunoprecipitation experiments were performed to identify specific protein O-GlcNAcylation targets. Because the initial omics analyses suggested that enzymes involved in fatty acid oxidation (FAO) can be a key target (FIGS. 14A-14L), this metabolic pathway was investigated. It was found that the mitochondrial fatty acid import protein CPT1b, and the fatty acid oxidation enzyme LCAD, displayed a non-significant increase in O-GlcNAcylation in HFpEF mice (FIG. 17A-17C). In both cases, treatment with adropin restored enzyme O-GlcNAcylation levels to those observed in chow mice (FIG. 17A-17C). Adropin-mediated changes in cardiac O-GlcNAcylation were examined to identify any effects on FAO enzyme activity; LCAD oxidation rates were significantly downregulated in the HFpEF mice compared to chow controls (FIG. 17D). Importantly, adropin treatment restored its activity in isolated cardiac tissues (FIG. 17D). Regression analysis demonstrated a significant negative correlation between LCAD O-GlcNAcylation status and enzyme activity (FIG. 17E), with no correlation between activity and total LCAD protein (FIG. 27), thereby suggesting that FAO enzyme O-GlcNAcylation decreases FAO activity in HFpEF. To confirm this, an in vitro assay was performed using recombinant LCAD, and found that OGT-catalyzed LCAD O-GlcNAcylation decreased enzyme activity relative to LCAD alone, or LCAD incubated with only the 0-GlcNAcylation substrate, UDP-GlcNAc (FIG. 17F). Interestingly, a significant increase in PLIN5 expression, a key lipid-droplet binding protein, was observed in the HFpEF mice compared to chow controls, indicating aberrant fatty acid utilization that was corrected upon adropin treatment (FIG. 28). Together, these findings suggest that long-term adropin treatment inhibits flux through HBP, limiting the deleterious O-GlcNAcylation of FAO enzymes, and thereby improving cardiac energy metabolism (FIG. 17G).

### Discussion

Diastolic dysfunction and liver injury coexist in the preclinical mouse model of heart failure with preserved ejection fraction. Markers of HFpEF severity significantly correlated NAFLD/MAFLD progression in the liver, with both diseases being characterized by metabolic dysregulation due to impaired fatty acid metabolism. Furthermore, HFpEF-driven disruptions to normal cardiac energy metabolism led to a shunt of glycolytic intermediates into the HBP, which drove increased cardiac protein 0-GlcNAcylation and inhibited fatty acid oxidation enzyme activity. Remarkably, long-term adropin treatment reversed this metabolic remodeling in the heart, leading to restored cardiac structure and diastolic function in the absence of liver protection. These findings are the first to report the long-term effects of adropin signaling on cardiac function and substrate flexibility in HFpEF, highlighting this pathway as a target for therapeutic interventions.

Cardio-hepatic interactions are complex and poorly understood, despite being commonly associated with metabolic diseases [35]. Heart failure progression is characterized by an increasing inability to meet the perfusion requirements of end organs such as the liver [36, 37]. As a highly vascularized organ with an intricate network of blood vessels, the liver is highly sensitive to any hemodynamic changes in the heart [38]. Liver disease is a common consequence of advanced heart failure, ranging from mild reversible liver injury to its most severe form, cardiac cirrhosis [39]. Several clinical studies have established the coexistence of diastolic dysfunction and hepatic steatosis [17-19, 40, 41], where the prevalence of NAFLD/MAFLD in HFpEF patients reached up to 50% [42]. However, there is limited evidence of this relationship in preclinical mouse models, thereby hindering the exploration of the pathophysiological mechanisms linking the two diseases. HFpEF severity and NAFLD/MAFLD were significantly correlated (FIG. 13A-13V), and differential expression of the shared genes Ces2a, Cyp3a11, and Elovl3 (FIGS. 14A-14L) suggested that fatty acid dysregulation is a central mechanism underlying the cardio-hepatic interactions. Interestingly, deletion of Ces2a [43, 44] or Cyp3a11 [45-47] has been implicated in the development of NAFLD/MAFLD, while Elovl3 expression has been shown to be dispensable for hepatic lipid homeostasis [48]. While these genes have known function in NAFLD/MAFLD progression, their roles in cardiac metabolism have yet to be fully elucidated. Recent studies have shown increased serum lipid metabolites in HFpEF patients with biopsy-proven NAFLD/MAFLD [49], and there were similar changes in human myocardial APOA1 levels [50] observed in HFpEF mouse hearts (FIG. 14A-14L), suggesting a significant role of fatty acid metabolism in the cardio-hepatic axis. Furthermore, untargeted metabolomics studies demonstrated that HFpEF accelerated flux into non-oxidative pathways such as methylglyoxal degradation (FIG. 14A-14L), which suggests advanced end glycation end products (AGE) accumulation, a major pathological feature that exacerbates tissue dysfunction in both heart failure [51, 52] and NAFLD/MAFLD [53, 53]. These studies show that impaired energy substrate utilization underlies the association between HFpEF and NAFLD/MAFLD.

Studies on adropin signaling in cardiometabolic disease have focused on its effects on fuel energy metabolism, with limited exploration of its role on myocardial structure and function. Short-term adropin treatment improved cardiac work and efficiency ex vivo, which was accompanied by enhanced insulin signaling in lean mice [32]. In contrast, exposure to a high-fat diet inhibited acute adropin-driven improvements in contractile function, and insulin stimulation failed to compensate for this impaired cardiac contractility in diet-induced obese mice ex vivo [33]. Chronic adropin treatment in vivo restores cardiac function, specifically left ventricular diastolic function, in the failing diabetic myocardium of mice (FIG. 15A-15T). Diastolic dysfunction is a hallmark of not only HFpEF, but also diabetic cardiomyopathy (DCM), where relaxation and filling pressure of the left ventricle is impaired [57]. Abnormalities of LV passive elasticity are caused by structural impediments, such as increased cardiomyocyte diameter and extracellular matrix deposition, which contribute to diastolic dysfunction [58]. Adropin treatment attenuates LV hypertrophy and fibrosis (FIG. 15A-15T), which is indicative of improved structural remodeling. In addition, transcriptomic analysis demonstrated increases in pro-fibrotic pathways as similarly seen in HFpEF patients [59] which was attenuated upon adropin treatment (FIG. 26), providing further evidence of restored myocardial structure and function.

These data report the transcriptomic and metabolomic signature of the HFD+L-NAME HFpEF model (FIG. 14A-14L), providing preclinical evidence of known mechanisms underlying the pathogenesis of cardiometabolic HFpEF in patients, such as fuel substrate inflexibility [50]. The ability of adropin treatment to regulate cardiac substrate utilization was independent of the inhibitory effects of PDK4 or GCN5L1 on pyruvate dehydrogenase activity. Adropin treatment reduces flux through the HBP, which inhibits cardiac protein 0-GlcNAcylation to promote fatty acid oxidation (FIGS. 16A-16I and 17A-17G). The HBP is a non-oxidative branch of glycolysis where the rate-limiting enzyme, GFAT, uses fructose-6-phosphate and glutamine to catalyze its conversion into glucosamine-6-phosphate, which is subsequently metabolized through a series of reactions utilizing substrates from major metabolic pathways to generate the end-product UDP-GlcNAc (FIGS. 4 and 5) [60]. UDP-GlcNAc serves as the co-factor for the attachment of an O-linked P-N-acetyl glucosamine moiety (O-GlcNAc) to serine and threonine residues of nuclear, cytoplasmic, and mitochondrial proteins, using 2-3% of total cellular glucose in the heart [61].

The dynamic on and off cycling of the O-GlcNAc modification is regulated by the activity of two enzymes, O-GlcNAc transferase (OGT) and O-GlcNAcase (OGA), respectively [75]. Several studies have implicated aberrant regulation of the HBP, and consequent maladaptive protein O-GlcNAcylation, in heart failure [60, 61, 75]. These studies have identified changes in OGT and OGA enzyme expression as the predominant mechanism that triggers excessive protein O-GlcNAcylation in diabetic hearts. However, these data show that changes in substrate entry from amino acid metabolism (glutamine), fatty acid metabolism (coenzyme A), and nucleotide metabolism (uridine) increased UDP-GlcNAc availability to promote increased cardiac protein O-GlcNAcylation levels (FIG. 16A-16I). Notably, previous comparative proteomics studies revealed dysregulated mitochondrial O-GlcNAcylation in diabetic hearts [63], with key targets in fatty acid oxidation that have subsequently been linked to increased enzyme activity in pressure-overload hypertrophy [64]. Laczy et al. (2011) showed that increased cardiac O-GlcNAcylation was associated with a decrease in glucose oxidation and a stimulation of fatty acid oxidation ex vivo, through increased CD36 expression and its O-GlcNAcylation. A significant increase in CD36 protein abundance was observed, which was significantly attenuated by adropin treatment (FIG. 16A-16I). Two FAO enzymes (CPT1b and LCAD) were identified as targets of O-GlcNAcylation in HFpEF mice, and adropin treatment showed moderate inhibition of the modification of these targets (FIG. 17A-17G). Interestingly, LCAD O-GlcNAcylation was associated with decreased cardiac enzyme activity in HFpEF mouse hearts, which was restored upon adropin treatment (FIG. 17A-17G). The inhibitory effect of O-GlcNAcylation on LCAD enzyme activity was confirmed in vitro, further suggesting that adropin treatment augments FAO activity via this mechanism.

In summary, a significant association between HFpEF and NAFLD/MAFLD was observed in a preclinical mouse model, with underlying links likely due to dysregulated lipid metabolism. Robust evidence is provided showing that prolonged adropin treatment restores fuel substrate metabolism to improve cardiac diastolic function and prevents structural remodeling in HFpEF. This study provides the first preclinical evidence that treatment with recombinant adropin represents a novel therapeutic avenue in HFpEF.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and compositions of matter, means, methods and steps described in the specification. As one of ordinary skill in the art will readily appreciate from the present disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps. Various patents, patent applications, publications, product descriptions, protocols, and sequence accession numbers are cited throughout this application, this present disclosures of which are incorporated herein by reference in their entireties for all purposes.

### REFERENCES

1. Heidenreich, P. A., Bozkurt, B., Aguilar, D., Allen, L. A., Byun, J. J., Colvin, M. M., Deswal, A., Drazner, M. H., Dunlay, S. M., Evers, L. R., Fang, J. C., Fedson, S. E., Fonarow, G. C., Hayek, S. S., Hernandez, A. F., Khazanie, P., Kittleson, M. M., Lee, C. S., Link, M. S., Milano, C. A., . . . Yancy, C. W. (2022). 2022 AHA/ACC/HFSA Guideline for the Management of Heart Failure: A Report of the American College of Cardiology/American Heart Association Joint Committee on Clinical Practice Guidelines. Circulation, 145(18), e895-e1032.
2. Ciccarelli, M., Dawson, D., Falcao-Pires, I., Giacca, M., Hamdani, N., Heymans, S., Hooghiemstra, A., Leeuwis, A., Hermkens, D., Tocchetti, C. G., van der Velden, J., Zacchigna, S., & Thum, T. (2021). Reciprocal organ interactions during heart failure: a position paper from the ESC Working Group on Myocardial Function. Cardiovascular research, 117(12), 2416-2433.
3. Redfield, M. M., & Borlaug, B. A. (2023). Heart Failure With Preserved Ejection Fraction: A Review. JAMA, 329(10), 827-838.
4. Shah, S. J., Borlaug, B. A., Kitzman, D. W., McCulloch, A. D., Blaxall, B. C., Agarwal, R., Chirinos, J. A., Collins, S., Deo, R. C., Gladwin, M. T., Granzier, H., Hummel, S. L., Kass, D. A., Redfield, M. M., Sam, F., Wang, T. J., Desvigne-Nickens, P., & Adhikari, B. B. (2020). Research Priorities for Heart Failure With Preserved Ejection Fraction: National Heart, Lung, and Blood Institute Working Group Summary. Circulation, 141(12), 1001-1026.
5. Shah, S. J., Kitzman, D. W., Borlaug, B. A., van Heerebeek, L., Zile, M. R., Kass, D. A., & Paulus, W. J. (2016). Phenotype-Specific Treatment of Heart Failure With Preserved Ejection Fraction: A Multiorgan Roadmap. Circulation, 134(1), 73-90.
6. Wong, C. M., Wang, Y., Lee, J. T., Huang, Z., Wu, D., Xu, A., & Lam, K. S. (2014). Adropin is a brain membrane-bound protein regulating physical activity via the NB-3/Notch signaling pathway in mice. The Journal of biological chemistry, 289(37), 25976-25986.
7. Kumar, K. G., Trevaskis, J. L., Lam, D. D., Sutton, G. M., Koza, R. A., Chouljenko, V. N., Kousoulas, K. G., Rogers, P. M., Kesterson, R. A., Thearle, M., Ferrante, A. W., Jr, Mynatt, R. L., Burris, T. P., Dong, J. Z., Halem, H. A., Culler, M. D., Heisler, L. K., Stephens, J. M., & Butler, A. A. (2008). Identification of adropin as a secreted factor linking dietary macronutrient intake with energy homeostasis and lipid metabolism. Cell metabolism, 8(6), 468-481.
8. Ganesh Kumar, K., Zhang, J., Gao, S., Rossi, J., McGuinness, O. P., Halem, H. H., Culler, M. D., Mynatt, R. L., & Butler, A. A. (2012). Adropin deficiency is associated with increased adiposity and insulin resistance. Obesity (Silver Spring, Md.), 20(7), 1394-1402.
9. Jurrissen, T. J., Ramirez-Perez, F. I., Cabral-Amador, F. J., Soares, R. N., Pettit-Mee, R. J., Betancourt-Cortes, E. E., McMillan, N. J., Sharma, N., Rocha, H. N. M., Fujie, S., Morales-Quinones, M., Lazo-Fernandez, Y., Butler, A. A., Banerjee, S., Sacks, H. S., Ibdah, J. A., Parks, E. J., Rector, R. S., Manrique-Acevedo, C., Martinez-Lemus, L. A., . . . Padilla, J. (2022). Role of adropin in arterial stiffening associated with obesity and type 2 diabetes. American journal of physiology. Heart and circulatory physiology, 323(5), H879-H891.
10. Schiattarella, G. G., Altamirano, F., Tong, D., French, K. M., Villalobos, E., Kim, S. Y., Luo, X., Jiang, N., May, H. I., Wang, Z. V., Hill, T. M., Mammen, P. P. A., Huang, J., Lee, D. I., Hahn, V. S., Sharma, K., Kass, D. A., Lavandero, S., Gillette, T. G., & Hill, J. A. (2019). Nitrosative stress drives heart failure with preserved ejection fraction. Nature, 568(7752), 351-356.
11. Jurczak, M. J., Lee, H. Y., Birkenfeld, A. L., Jornayvaz, F. R., Frederick, D. W., Pongratz, R. L., Zhao, X., Moeckel, G. W., Samuel, V. T., Whaley, J. M., Shulman, G. I., & Kibbey, R. G. (2011). SGLT2 deletion improves glucose homeostasis and preserves pancreatic beta-cell function. Diabetes, 60(3), 890-898.
12. Kleiner, D. E., Brunt, E. M., Van Natta, M., Behling, C., Contos, M. J., Cummings, O. W., Ferrell, L. D., Liu, Y. C., Torbenson, M. S., Unalp-Arida, A., Yeh, M., McCullough, A. J., Sanyal, A. J., & Nonalcoholic Steatohepatitis Clinical Research Network (2005). Design and validation of a histological scoring system for nonalcoholic fatty liver disease. Hepatology (Baltimore, Md.), 41(6), 1313-1321.
13. Thapa, D., Zhang, M., Manning, J. R., Guimaraes, D. A., Stoner, M. W., O'Doherty, R. M., Shiva, S., & Scott, I. (2017). Acetylation of mitochondrial proteins by GCN5L1 promotes enhanced fatty acid oxidation in the heart. American journal of physiology. Heart and circulatory physiology, 313(2), H265-H274.
14. Eslam, M., Newsome, P. N., Sarin, S. K., Anstee, Q. M., Targher, G., Romero-Gomez, M., Zelber-Sagi, S., Wai-Sun Wong, V., Dufour, J. F., Schattenberg, J. M., Kawaguchi, T., Arrese, M., Valenti, L., Shiha, G., Tiribelli, C., Yki-Jarvinen, H., Fan, J. G., Gronbok, H., Yilmaz, Y., Cortez-Pinto, H., . . . George, J. (2020). A new definition for metabolic dysfunction-associated fatty liver disease: An international expert consensus statement. Journal of hepatology, 73(1), 202-209.
15. Shah, S. J., Katz, D. H., Selvaraj, S., Burke, M. A., Yancy, C. W., Gheorghiade, M., Bonow, R. O., Huang, C. C., & Deo, R. C. (2015). Phenomapping for novel classification of heart failure with preserved ejection fraction. Circulation, 131(3), 269-279.
16. Salah, H. M., Pandey, A., Soloveva, A., Abdelmalek, M. F., Diehl, A. M., Moylan, C. A., Wegermann, K., Rao, V. N., Hernandez, A. F., Tedford, R. J., Parikh, K. S., Mentz, R. J., McGarrah, R. W., & Fudim, M. (2021). Relationship of Nonalcoholic Fatty Liver Disease and Heart Failure With Preserved Ejection Fraction. JACC. Basic to translational science, 6(11), 918-932.
17. Mantovani, A., Pernigo, M., Bergamini, C., Bonapace, S., Lipari, P., Pichiri, I., Bertolini, L., Valbusa, F., Barbieri, E., Zoppini, G., Bonora, E., & Targher, G. (2015). Nonalcoholic Fatty Liver Disease Is Independently Associated with Early Left Ventricular Diastolic Dysfunction in Patients with Type 2 Diabetes. PloS one, 10(8), e0135329.
18. Lee, H., Kim, G., Choi, Y. J., Huh, B. W., Lee, B. W., Kang, E. S., Cha, B. S., Lee, E. J., Lee, Y. H., & Huh, K. B. (2020). Association between Non-Alcoholic Steatohepatitis and Left Ventricular Diastolic Dysfunction in Type 2 Diabetes Mellitus. Diabetes & metabolism journal, 44(2), 267-276.
19. Wang, S., Zhang, X., Zhang, Q., Zhang, B. & Zhao, L. Is non-alcoholic fatty liver disease a sign of left ventricular diastolic dysfunction in patients with type 2 diabetes mellitus? A systematic review and meta-analysis. BMJ Open Diabetes Research & Care 11, e003198 (2023).
20. Lautamaki, R., Borra, R., Iozzo, P., Komu, M., Lehtimaki, T., Salmi, M., Jalkanen, S., Airaksinen, K.E., Knuuti, J., Parkkola, R., & Nuutila, P. (2006). Liver steatosis coexists with myocardial insulin resistance and coronary dysfunction in patients with type 2 diabetes. American journal of physiology. Endocrinology and metabolism, 291(2), E282-E290.
21. Rijzewijk, L. J., Jonker, J. T., van der Meer, R. W., Lubberink, M., de Jong, H. W., Romijn, J. A., Bax, J. J., de Roos, A., Heine, R. J., Twisk, J. W., Windhorst, A. D., Lammertsma, A. A., Smit, J. W., Diamant, M., & Lamb, H. J. (2010). Effects of hepatic triglyceride content on myocardial metabolism in type 2 diabetes. Journal of the American College of Cardiology, 56(3), 225-233. https://doi.org/10.1016/j.jacc.2010.02.049
22. Lee, M., Kim, K. J., Chung, T. H., Bae, J., Lee, Y. H., Lee, B. W., Cha, B. S., Yun, M., & Kang, E. S. (2021). Nonalcoholic fatty liver disease, diastolic dysfunction, and impaired myocardial glucose uptake in patients with type 2 diabetes. Diabetes, Obesity and Metabolism. 23, 1041-1051 (2021).
23. Cassidy, S., Hallsworth, K., Thoma, C., MacGowan, G. A., Hollingsworth, K. G., Day, C. P., Taylor, R., Jakovljevic, D. G., & Trenell, M. I. (2015). Cardiac structure and function are altered in type 2 diabetes and non-alcoholic fatty liver disease and associate with glycemic control. Cardiovascular diabetology, 14, 23.
24. Peng, D., Yu, Z., Wang, M., Shi, J., Sun, L., Zhang, Y., Zhao, W., Chen, C., Tang, J., Wang, C., Ni, J., Wen, W., & Jiang, J. (2022). Association of Metabolic Dysfunction-Associated Fatty Liver Disease With Left Ventricular Diastolic Function and Cardiac Morphology. Frontiers in endocrinology, 13, 935390.
25. Dong, Y., Cui, H., Sun, L., Wang, Y., Li, Y., Chang, W., Li, G., & Huang, D. (2020). Assessment of left ventricular function in type 2 diabetes mellitus patients with non-alcoholic fatty liver disease using three-dimensional speckle-tracking echocardiography. Anatolian journal of cardiology, 23(1), 41-48.
26. Yong, J. N., Ng, C. H., Lee, C. W., Chan, Y. Y., Tang, A. S. P., Teng, M., Tan, D. J. H., Lim, W. H., Quek, J., Xiao, J., Chin, Y. H., Foo, R., Chan, M., Lin, W., Noureddin, M., Siddiqui, M. S., Muthiah, M. D., Sanyal, A., & Chew, N. W. S. (2022). Non-alcoholic fatty liver disease association with structural heart, systolic and diastolic dysfunction: a meta-analysis. Hepatology international, 16(2), 269-281.
27. Chen, R. B., Wang, Q. Y., Wang, Y. Y., Wang, Y. D., Liu, J. H., Liao, Z. Z., & Xiao, X. H. (2023). Feeding-induced hepatokines and crosstalk with multi-organ: A novel therapeutic target for Type 2 diabetes. Frontiers in endocrinology, 14, 1094458.
28. Mushala, B. A. S., & Scott, I. (2021). Adropin: a hepatokine modulator of vascular function and cardiac fuel metabolism. American journal of physiology. Heart and circulatory physiology, 320(1), H238-H244.
29. Gao, S., McMillan, R. P., Zhu, Q., Lopaschuk, G. D., Hulver, M. W., & Butler, A. A. (2015). Therapeutic effects of adropin on glucose tolerance and substrate utilization in diet-induced obese mice with insulin resistance. Molecular metabolism, 4(4), 310-324.
30. Thapa, D., Xie, B., Manning, J. R., Zhang, M., Stoner, M. W., Huckestein, B. R., Edmunds, L. R., Zhang, X., Dedousis, N. L., O'Doherty, R. M., Jurczak, M. J., & Scott, I. (2019). Adropin reduces blood glucose levels in mice by limiting hepatic glucose production. Physiological reports, 7(8), e14043.
31. Thapa, D., Xie, B., Zhang, M., Stoner, M. W., Manning, J. R., Huckestein, B. R., Edmunds, L. R., Mullett, S. J., McTiernan, C. F., Wendell, S. G., Jurczak, M. J., & Scott, I. (2019). Adropin treatment restores cardiac glucose oxidation in pre-diabetic obese mice. Journal of molecular and cellular cardiology, 129, 174-178.
32. Altamimi, T. R., Gao, S., Karwi, Q. G., Fukushima, A., Rawat, S., Wagg, C. S., Zhang, L., & Lopaschuk, G. D. (2019). Adropin regulates cardiac energy metabolism and improves cardiac function and efficiency. Metabolism: clinical and experimental, 98, 37-48.
33. Thapa, D., Xie, B., Mushala, B. A. S., Zhang, M., Manning, J. R., Bugga, P., Stoner, M. W., Jurczak, M. J., & Scott, I. (2022). Diet-induced obese mice are resistant to improvements in cardiac function resulting from short-term adropin treatment. Current research in physiology, 5, 55-62.
34. Chen, X., Xue, H., Fang, W., Chen, K., Chen, S., Yang, W., Shen, T., Chen, X., Zhang, P., & Ling, W. (2019). Adropin protects against liver injury in nonalcoholic steatohepatitis via the Nrf2 mediated antioxidant capacity. Redox biology, 21, 101068.
35. Zhang, Y., & Fang, X. M. (2021). Hepatocardiac or Cardiohepatic Interaction: From Traditional Chinese Medicine to Western Medicine. Evidence-based complementary and alternative medicine:eCAM, 2021, 6655335.
36. Fang, J. C., Ewald, G. A., Allen, L. A., Butler, J., Westlake Canary, C. A., Colvin-Adams, M., Dickinson, M. G., Levy, P., Stough, W. G., Sweitzer, N. K., Teerlink, J. R., Whellan, D. J., Albert, N. M., Krishnamani, R., Rich, M. W., Walsh, M. N., Bonnell, M. R., Carson, P. E., Chan, M. C., Dries, D. L., . . . Heart Failure Society of America Guidelines Committee (2015). Advanced (stage D) heart failure: a statement from the Heart Failure Society of America Guidelines Committee. Journal of cardiac failure, 21(6), 519-534.
37. Samsky, M. D., Patel, C. B., DeWald, T. A., Smith, A. D., Felker, G. M., Rogers, J. G., & Hernandez, A. F. (2013). Cardiohepatic interactions in heart failure: an overview and clinical implications. Journal of the American College of Cardiology, 61(24), 2397-2405.
38. Xanthopoulos, A., Starling, R. C., Kitai, T., & Triposkiadis, F. (2019). Heart Failure and Liver Disease: Cardiohepatic Interactions. JACC. Heart failure, 7(2), 87-97.
39. Pendyal, A., & Gelow, J. M. (2016). Cardiohepatic Interactions: Implications for Management in Advanced Heart Failure. Heart failure clinics, 12(3), 349-361.
40. VanWagner, L. B., Wilcox, J. E., Colangelo, L. A., Lloyd-Jones, D. M., Carr, J. J., Lima, J. A., Lewis, C. E., Rinella, M. E., & Shah, S. J. (2015). Association of nonalcoholic fatty liver disease with subclinical myocardial remodeling and dysfunction: A population-based study. Hepatology (Baltimore, Md.), 62(3), 773-783.
41. Fudim, M., Zhong, L., Patel, K. V., Khera, R., Abdelmalek, M. F., Diehl, A. M., McGarrah, R. W., Molinger, J., Moylan, C. A., Rao, V. N., Wegermann, K., Neeland, I. J., Halm, E. A., Das, S. R., & Pandey, A. (2021). Nonalcoholic Fatty Liver Disease and Risk of Heart Failure Among Medicare Beneficiaries. Journal of the American Heart Association, 10(22), e021654.
42. Miller, A., McNamara, J., Hummel, S. L., Konerman, M. C., & Tincopa, M. A. (2020). Prevalence and staging of non-alcoholic fatty liver disease among patients with heart failure with preserved ejection fraction. Scientific reports, 10(1), 12440.
43. Chalhoub, G., Jamnik, A., Pajed, L., Kolleritsch, S., Hois, V., Bagaric, A., Prem, D., Tilp, A., Kolb, D., Wolinski, H., Taschler, U., Züllig, T., Rechberger, G. N., Fuchs, C., Trauner, M., Schoiswohl, G., & Haemmerle, G. (2023). Carboxylesterase 2a deletion provokes hepatic steatosis and insulin resistance in mice involving impaired diacylglycerol and lysophosphatidylcholine catabolism. Molecular metabolism, 72, 101725.
44. Li, Y., Zalzala, M., Jadhav, K., Xu, Y., Kasumov, T., Yin, L., & Zhang, Y. (2016). Carboxylesterase 2 prevents liver steatosis by modulating lipolysis, endoplasmic reticulum stress, and lipogenesis and is regulated by hepatocyte nuclear factor 4 alpha in mice. Hepatology (Baltimore, Md.), 63(6), 1860-1874.
45. Woolsey, S. J., Mansell, S. E., Kim, R. B., Tirona, R. G., & Beaton, M. D. (2015). CYP3A Activity and Expression in Nonalcoholic Fatty Liver Disease. Drug metabolism and disposition: the biological fate of chemicals, 43(10), 1484-1490.
46. Zeng, H., Lin, Y., Gong, J., Lin, S., Gao, J., Li, C., Feng, Z., Zhang, H., Zhang, J., Li, Y., & Yu, C. (2019). CYP3A suppression during diet-induced nonalcoholic fatty liver disease is independent of PXR regulation. Chemico-biological interactions, 308, 185-193.
47. Jamwal, R., de la Monte, S. M., Ogasawara, K., Adusumalli, S., Barlock, B. B., & Akhlaghi, F. (2018). Nonalcoholic Fatty Liver Disease and Diabetes Are Associated with Decreased CYP3A4 Protein Expression and Activity in Human Liver. Molecular pharmaceutics, 15(7), 2621-2632.
48. Qin, Z., Wang, P., Chen, W., Wang, J. R., Ma, X., Zhang, H., Zhang, W. J., & Wei, C. (2023). Hepatic ELOVL3 is dispensable for lipid metabolism in mice. Biochemical and biophysical research communications, 658, 128-135.
49. Wegermann, K., Fudim, M., Henao, R., Howe, C. F., McGarrah, R., Guy, C., Abdelmalek, M. F., Diehl, A. M., & Moylan, C. A. (2023). Serum Metabolites Are Associated With HFpEF in Biopsy-Proven Nonalcoholic Fatty Liver Disease. Journal of the American Heart Association, 12(14), e029873.
50. Hahn, V. S., Petucci, C., Kim, M. S., Bedi, K. C., Jr, Wang, H., Mishra, S., Koleini, N., Yoo, E. J., Margulies, K. B., Arany, Z., Kelly, D. P., Kass, D. A., & Sharma, K. (2023). Myocardial Metabolomics of Human Heart Failure With Preserved Ejection Fraction. Circulation, 147(15), 1147-1161.
51. Bodiga, V. L., Eda, S. R. & Bodiga, S. Advanced glycation end products: role in pathology of diabetic cardiomyopathy. Heart Fail Rev 19, 49-63 (2014).
52. Arshi, B., Chen, J., Ikram, M. A., Zillikens, M. C., & Kavousi, M. (2023). Advanced glycation end-products, cardiac function and heart failure in the general population: The Rotterdam Study. Diabetologia, 66(3), 472-481.
53. Spanos, C., Maldonado, E.M., Fisher, C. P. et al. Proteomic identification and characterization of hepatic glyoxalase 1 dysregulation in non-alcoholic fatty liver disease. Proteome Sci 16, 4 (2018).
54. Sakasai-Sakai, A., Takata, T., Takino, Ji. et al. Impact of intracellular glyceraldehyde-derived advanced glycation end-products on human hepatocyte cell death. Sci Rep 7, 14282 (2017).
55. Beale, A. L., Meyer, P., Marwick, T. H., Lam, C. S. P., & Kaye, D. M. (2018). Sex Differences in Cardiovascular Pathophysiology: Why Women Are Overrepresented in Heart Failure With Preserved Ejection Fraction. Circulation, 138(2), 198-205.
56. Chen, X., Sun, X., Shen, T., Chen, Q., Chen, S., Pang, J., Mi, J., Tang, Y., You, Y., Xu, H., & Ling, W. (2020). Lower adropin expression is associated with oxidative stress and severity of nonalcoholic fatty liver disease. Free radical biology & medicine, 160, 191-198.
57. Jia, G., Hill, M. A., & Sowers, J. R. (2018). Diabetic Cardiomyopathy: An Update of Mechanisms Contributing to This Clinical Entity. Circulation research, 122(4), 624-638.
58. Aurigemma, G. P., Zile, M. R. & Gaasch, W. H. Contractile behavior of the left ventricle in diastolic heart failure: with emphasis on regional systolic function. Circulation 113, 296-304 (2006).
59. Ye, B., Bradshaw, A. D., Abrahante, J. E., Dragon, J. A., Häußler, T. N., Bell, S. P., Hirashima, F., LeWinter, M., Zile, M. R., & Meyer, M. (2023). Left Ventricular Gene Expression in Heart Failure With Preserved Ejection Fraction-Profibrotic and Proinflammatory Pathways and Genes. Circulation. Heart failure, 16(8), e010395.
60. Umapathi, P., et al. Excessive O-GlcNAcylation Causes Heart Failure and Sudden Death. Circulation 143, 1687-1703 (2021). 154. Tran, D. H., et al. Chronic activation of hexosamine biosynthesis in the heart triggers pathological cardiac remodeling. Nature Communications 11, 1771 (2020). 155.
61. Tran, D. H., May, H. I., Li, Q. et al. Chronic activation of hexosamine biosynthesis in the heart triggers pathological cardiac remodeling. Nat Commun 11, 1771 (2020).
62. Prakoso, D., et al. Fine-tuning the cardiac O-GlcNAcylation regulatory enzymes governs the functional and structural phenotype of the diabetic heart. Cardiovascular Research 118, 212-225 (2021). 156.
63. Ma, J., Banerjee, P., Whelan, S. A., Liu, T., Wei, A. C., Ramirez-Correa, G., McComb, M. E., Costello, C. E., O'Rourke, B., Murphy, A., & Hart, G. W. (2016). Comparative Proteomics Reveals Dysregulated Mitochondrial O-GlcNAcylation in Diabetic Hearts. Journal of proteome research, 15(7), 2254-2264.
64. Zhu, W. Z., Palazzo, T., Zhou, M., Ledee, D., Olson, H. M., Pasa-Tolid, L., & Olson, A. K. (2022). First comprehensive identification of cardiac proteins with putative increased O-GlcNAc levels during pressure overload hypertrophy. PloS one, 17(10), e0276285.
65. Laczy, B., Fülöp, N., Onay-Besikci, A., Des Rosiers, C. & Chatham, J. C. Acute Regulation of Cardiac Metabolism by the Hexosamine Biosynthesis Pathway and Protein OGlcNAcylation. PLOS ONE 6, e18417 (2011).
66. Gori, M., Lam, C. S., Gupta, D. K., Santos, A. B., Cheng, S., Shah, A. M., Claggett, B., Zile, M. R., Kraigher-Krainer, E., Pieske, B., Voors, A. A., Packer, M., Bransford, T., Lefkowitz, M., McMurray, J. J., Solomon, S. D., & PARAMOUNT Investigators (2014). Sex-specific cardiovascular structure and function in heart failure with preserved ejection fraction. European journal of heart failure, 16(5), 535-542.
67. Beale, A. L., Meyer, P., Marwick, T. H., Lam, C. S. P., & Kaye, D. M. (2018). Sex Differences in Cardiovascular Pathophysiology: Why Women Are Overrepresented in Heart Failure With Preserved Ejection Fraction. Circulation, 138(2), 198-205.
68. Duca, F., Zotter-Tufaro, C., Kammerlander, A. A., Aschauer, S., Binder, C., Mascherbauer, J., & Bonderman, D. (2018). Gender-related differences in heart failure with preserved ejection fraction. Scientific reports, 8(1), 1080.
69. Lindman, B. R., Davila-Roman, V. G., Mann, D. L., McNulty, S., Semigran, M. J., Lewis, G. D., de las Fuentes, L., Joseph, S. M., Vader, J., Hernandez, A. F., & Redfield, M. M. (2014). Cardiovascular phenotype in HFpEF patients with or without diabetes: a RELAX trial ancillary study. Journal of the American College of Cardiology, 64(6), 541-549.
70. Lagou, V., Magi, R., Hottenga, J. J. et al. Sex-dimorphic genetic effects and novel loci for fasting glucose and insulin variability. Nat Commun 12, 24 (2021).
71. Cao, Y., Vergnes, L., Wang, Y. C., Pan, C., Chella Krishnan, K., Moore, T. M., Rosa-Garrido, M., Kimball, T. H., Zhou, Z., Charugundla, S., Rau, C. D., Seldin, M. M., Wang, J., Wang, Y., Vondriska, T. M., Reue, K., & Lusis, A. J. (2022). Sex differences in heart mitochondria regulate diastolic dysfunction. Nature communications, 13(1), 3850.
72. Stokar, J., Gurt, I., Cohen-Kfir, E., Yakubovsky, O., Hallak, N., Benyamini, H., Lishinsky, N., Offir, N., Tam, J., & Dresner-Pollak, R. (2022). Hepatic adropin is regulated by estrogen and contributes to adverse metabolic phenotypes in ovariectomized mice. Molecular metabolism, 60, 101482.
73. Kittleson, M. M., Panjrath, G. S., Amancherla, K., Davis, L. L., Deswal, A., Dixon, D. L., Januzzi, J. L., Jr, & Yancy, C. W. (2023). 2023 ACC Expert Consensus Decision Pathway on Management of Heart Failure With Preserved Ejection Fraction: A Report of the American College of Cardiology Solution Set Oversight Committee. Journal of the American College of Cardiology, 81(18), 1835-1878.
74. Kosiborod, M. N., Abildstrom, S. Z., Borlaug, B. A., Butler, J., Rasmussen, S., Davies, M., Hovingh, G. K., Kitzman, D. W., Lindegaard, M. L., Møller, D. V., Shah, S. J., Treppendahl, M. B., Verma, S., Abhayaratna, W., Ahmed, F. Z., Chopra, V., Ezekowitz, J., Fu, M., Ito, H., Lelonek, M., . . . STEP-HFpEF Trial Committees and Investigators (2023). Semaglutide in Patients with Heart Failure with Preserved Ejection Fraction and Obesity. The New England journal of medicine, 10.1056/NEJMoa2306963. Advance online publication.
75. Prakoso, D., Lim, S. Y., Erickson, J. R., Wallace, R. S., Lees, J. G., Tate, M., Kiriazis, H., Donner, D. G., Henstridge, D. C., Davey, J. R., Qian, H., Deo, M., Parry, L. J., Davidoff, A. J., Gregorevic, P., Chatham, J. C., De Blasio, M. J., & Ritchie, R. H. (2022). Fine-tuning the cardiac O-GlcNAcylation regulatory enzymes governs the functional and structural phenotype of the diabetic heart. Cardiovascular research, 118(1), 212-225.

What is claimed is:

1. A method for preventing and/or treating a subject having cardiometabolic disease, comprising administering a therapeutically effective amount of an Adropin-based therapy.

2. The method of claim 1, wherein the Adropin-based therapy is a composition comprising a virus, wherein the virus comprises a heterologous nucleic acid encoding an Energy Homeostasis-associated (ENHO) gene.

3. The method of claim 2, wherein the virus is administered into the liver of the subject.

4. The method of claim 2, wherein the virus is administered to hepatocytes of the subject.

5. The method of claim 2, wherein the heterologous nucleic acid is operably linked to a promoter selected from the group consisting of thyroxine binding globulin (TBG), albumin (ALB), hepatitis virus (HBV), alpha-1 antitrypsin (AAT), and human cytomegalovirus (CMV).

6. The method of claim 2, wherein the virus is an adenoviral associated virus (AAV).

7. The method of claim 6, wherein the AAV isotype is selected from the group consisting of AAV1, AAV2, AAV3, AAV6, AAV6.2, AAV7, AAV8, AAV9, AAVrh10, AAV/DJ, and AAV/DJ8.

8. The method of claim 2, wherein the ENHO gene comprises SEQ ID NO.: 3.

9. The method of claim 1, wherein the Adropin-based therapy is a recombinant Adropin peptide.

10. The method of claim 9, wherein the recombinant Adropin peptide comprises SEQ ID NO.: 1 or SEQ ID NO.: 2.

11. The method of claim 1, wherein the Adropin-based therapy is administered intravenously, intraperitoneally, or subcutaneously.

12. The method of claim 9, wherein the recombinant Adropin peptide is administered in an amount between about 1 ng/kg to about 200 mg/kg.

13. The method of claim 1, wherein the cardiometabolic disease is selected from the group consisting of heart failure with preserved ejection fraction (HFpEF), type-2 diabetes, hypertension, or non-alcoholic fatty liver disease (NAFLD).

14. A pharmaceutical composition comprising a therapeutically effective amount of an Adropin-based therapy.

15. The pharmaceutical composition of claim 14, further comprising a pharmaceutically acceptable carrier.

16. The pharmaceutical composition of claim 14, wherein the Adropin-based therapy is a virus, wherein the virus comprises a heterologous nucleic acid encoding an ENHO gene.

17. The method of claim 16, wherein the virus is an AAV.

18. The pharmaceutical composition of claim 16, wherein the ENHO gene comprises SEQ ID NO.: 3.

19. The pharmaceutical composition of claim 14, wherein the Adropin-based therapy is a recombinant Adropin peptide.

20. The pharmaceutical composition of claim 19, wherein the recombinant Adropin peptide comprises SEQ ID NO.: 1 or SEQ ID NO.: 2.
